(19) United States
(12) Patent Application Publication
(10) Pub. No.: US 2024/0279332 A1
(43) Pub. Date: Aug. 22, 2024

(54) Treatment of Cardiovascular Disease with TREM-1 Antigen Binding Proteins

(71) Applicant: AMGEN INC., Thousand Oaks, CA (US)

(72) Inventors: Susan SHETTERLY, Thousand Oaks, CA (US); Simon Mark JACKSON, Thousand Oaks, CA (US)

(21) Appl. No.: 18/571,614

(22) PCT Filed: Jun. 24, 2022
(86) PCT No.: PCT/US22/34834

§ 371 Date: Dec. 18, 2023

Related U.S. Application Data
(60) Provisional application No. 63/353,223, filed on Jun. 17, 2022.
(60) Provisional application No. 63/215,260, filed on Jun. 25, 2021.

(51) Int. Cl. C07K 16/28; A61K 45/06

(52) U.S. Cl. CPC C07K 16/2803; A61K 45/06; C07K 2317/24; C07K 2317/52; C07K 2317/565; C07K 2317/567

(57) ABSTRACT

The present disclosure relates, in general, to methods of treating cardiovascular disease, such as atherosclerosis or myocardial infarction, using antigen binding proteins that bind to TREM-1, and compositions thereof.

### CROSS-REFERENCE TO RELATED APPLICATION(S)

The present application claims the priority benefit of U.S. Provisional Patent Application No. 63/215,260, filed Jun. 25, 2021 and U.S. Provisional Patent Application No. 63/353,223, filed Jun. 17, 2022, which are hereby incorporated by reference in their entirety.

### FIELD OF THE DISCLOSURE

The present disclosure relates to antigen binding moieties specific for, TrigGering Receptor Expressed on Myeloid cells 1 (TREM-1) and compositions thereof, for the treatment of cardiovascular conditions, such as atherosclerosis or myocardial infarction (MI).

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “56967P2_Seqlisting.txt”, which was created on Jun. 17, 2022 and is 1368364 bytes in size. The subject matter of the Sequence Listing is incorporated herein in its entirety by reference.

### BACKGROUND OF THE DISCLOSURE

Triggering receptor expressed on myeloid cells 1 (TREM1, TREM-1, CD354) is an Ig family member expressed in neutrophil, monocyte and macrophage cells. TREM-1 is a mediator of the myeloid cell immune response, Activation through TREM-1 induces inflammatory cytokines, including IL-8, MCP/CCL2, and tumor necrosis factor alpha (TNFα). PGLYRP1 (Peptidoglycan recognition protein 1) has recently been reported as a ligand for TREM-1 (Read, J. Immunol. 194: 1417-1421, 2015), and potential other ligands remain to be determined. TREM-1 has been implicated in upregulating the inflammatory response in sepsis (Ford et al. Curr Op Immunol. 2009; 21:38-46), and blockade of TREM-1 activity by TREM-1-Fc or antagonistic peptide (e.g. LP17) reduced the endotoxic effects in septic animals (Ford, supra, Qian et al., J.Int J Clin Exp Med. 2014 7(7):1650-8). TREM-1 knockout mice are viable and protected from DSS colitis and T cell transfer colitis (Weber, PLoS Pathoq, 10(1):1003900, 2014).

Treatment with a TREM-1-Fc fusion improved survival and reduced TNFα induction after LPS challenge in mice (Bouchon, Nature, 410:1103-7, 2001). An anti-TREM-1 antibody was reported to reduce secretion of inflammatory cytokines from lamina propria cells isolated from IBD patients stimulated with TREM-1 agonist PGLYRP-1/peptidoglycan (Brynjolfsson et al., Inflamm Bowel Dis 22(8):1803-11, 2016). Additionally, nangibotide, achemically synthesized 12 L-amino acid peptide, derived from the protein TREM-like transcript-1 (TLT-1), has been investigated as a treatment for septic shock (Cuvier et al., Br J Clin Pharmacol. 2018 October; 84(10): 2270-2279). While TREM-1 is primarily expressed on monocyte/macrophage and neutrophils, during inflammation it is also found on bronchial, corneal, gastric epithelial cells, and hepatic endothelial cells.

### SUMMARY OF THE DISCLOSURE

The present disclosure provides antigen binding proteins specific for human TrigGering Receptor Expressed on Myeloid cells 1 (TREM-1) and are useful in the treatment of cardiovascular diseases.

Provided herein is a method of treating cardiovascular disease (e.g., atherosclerosis or myocardial infarction) comprising administering to a subject in need thereof a therapeutically effective amount of an antigen binding protein that binds to TrigGering Receptor Expressed on Myeloid cells 1 (TREM-1), the antigen binding protein comprising:
a. a light chain variable domain comprising:
   i. a light chain CDR1 comprising an amino acid sequence selected from SEQ ID NOS: 10, 30, 50, 70, 90, 110, 130, 150, 170, 190, 210, 230, 250, 270 and 544;
   ii. a light chain CDR2 comprising an amino acid sequence selected from SEQ ID NOS: 11, 31, 51, 71, 91, 111, 131, 151, 171, 191, 211, 231, 251, 271, and 545;
   iii. a light chain CDR3 comprising an amino acid sequence selected from SEQ ID NOS: 12, 32, 52, 72, 92, 112, 132, 152, 172, 192, 212, 232, 252, 272 and 546; and
b. a heavy chain variable domain comprising:
   i. a heavy chain CDR1 comprising an amino acid sequence selected from SEQ ID NOS: 16, 36, 56, 76, 96, 116, 136, 156, 176, 196, 216, 236, 256, 276, and 550;
   ii. a heavy chain CDR2 comprising an amino acid sequence selected from SEQ ID NOS: 17, 37, 57, 77, 97, 117, 137, 157, 177, 197, 217, 237, 257, 277, and 551; and
   iii. a heavy chain CDR3 comprising an amino acid sequence selected from SEQ ID NOS: 18, 38, 58, 78, 98, 118, 138, 158, 178, 198, 218, 238, 258, 278, and 552.

In various embodiments, the antigen binding protein comprises:
a. the light chain CDR1 sequence set out in SEQ ID NO: 10, 30, 50, 90, 130, 150, or 270;
b. the light chain CDR2 sequence set out in SEQ ID NO: 11, 31, 51, 91, 131, 151, or 271;
c. the light chain CDR3 sequence set out in SEQ ID NO: 12, 32, 52, 92, 132, 152, or 272
d. the heavy chain CDR1 sequence set out in SEQ ID NO: 16, 36, 56, 96, 136, 156, or 276;
e. the heavy chain CDR2 sequence set out in SEQ ID NO: 17, 37, 57, 97, 137, 157, or 277; and
f. the heavy chain CDR3 sequence set out in SEQ ID NO: 18, 38, 58, 98, 138, 158, or 278.

In various embodiments, the antigen binding protein comprises:
a. the light chain CDR1 sequence set out in SEQ ID NO: 30 or 90;
b. the light chain CDR2 sequence set out in SEQ ID NO: 31 or 91;
c. the light chain CDR3 sequence set out in SEQ ID NO: 32 or 92;
d. the heavy chain CDR1 sequence set out in SEQ ID NO: 36 or 096;
e. the heavy chain CDR2 sequence set out in SEQ ID NO: 37 or 97; and
f. the heavy chain CDR3 sequence set out in SEQ ID NOS: 38 or 98.

Also contemplated are consensus sequences of the TREM-1 antibody/antigen binding protein heavy and light chain CDRs and/or variable region sequences disclosed herein. For example, in various embodiments, the TREM-1 antibody comprises an antigen binding domain comprising a sequence having a light chain variable region comprising a LCDR1 amino acid sequence selected from the group consisting of:
X₁ASQSX₂X₃X₄NLA (SEQ ID NO: 553), wherein X₁ is R or Q, wherein X₂ is V or I, wherein X₃ is N or S, and wherein X₄ is S, H, I, V or A;
QASX₁DIX₂X₃X₄LN (SEQ ID NO: 558), wherein X₁ is R or Q, wherein X₂ is R, S, N or F, wherein X₃ is K or N, and wherein X₄ is H, Y or D;

(SEQ ID NO: 566)
RASQSVNSNLA;
(SEQ ID NO: 567)
QASQDIRKHLN;
(SEQ ID NO: 568)
RASQDISSNLN;
(SEQ ID NO: 569)
QASQDIHLN;
(SEQ ID NO: 570)
RASQGIRKWLA
(SEQ ID NO: 571)
RASQSVNSNLA
and
(SEQ ID NO: 572)
SGDKLGERVS.

In various embodiments, the TREM-1 antigen binding protein comprises an antigen binding domain comprising a sequence having a light chain variable region comprising a LCDR2 amino acid sequence selected from the group consisting of:
GAX₁X₂RAT (SEQ ID NO: 554), wherein X₁ is S or Y, and wherein X₂ is T or I;
X₁X₂X₃X₄LET (SEQ ID NO: 560), wherein X₁ is D, G or H, wherein X₂ is A, V or T, wherein X₃ is S, A or Y, and wherein X₄ is T or N;

(SEQ ID NO: 573)
GASTRAT;
(SEQ ID NO: 574)
DASNLET;
and
(SEQ ID NO: 575)
AASRLQS.

In various embodiments, the TREM-1 antigen binding protein comprises an antigen binding domain comprising a sequence having a light chain variable region comprising a LCDR3 amino acid sequence selected from the group consisting of
QX₁X₂X₃X₄X₅X₆PX₇T (SEQ ID NO: 555); wherein X₁ is Q, H or E, wherein X₂ is F or Y, wherein X₃ is K, Y or I, wherein X₄ is N, T, L, I, or M; wherein X₅ is W, F, H or Y, wherein X₆ is absent or P; wherein X₇ is W, N, Y, H or L;
QX₁YX₃X₄X₅PX₆T (SEQ ID NO: 561), wherein X₁ is Q or H, wherein X₂ is D, A or G, wherein X₃ is N or K; wherein X₄ is L or I, and wherein X₅ is I or L;

(SEQ ID NO: 576)
QQFKNWPPT;
(SEQ ID NO: 577)
QHYDNLPIT;
(SEQ ID NO: 578)
LQAHGFPWT;
(SEQ ID NO: 579)
QQYDNLPLT
and
(SEQ ID NO: 580)
QFWPPWT.

In various embodiments, the TREM-1 antigen binding protein comprises an antigen binding domain comprising a sequence having a heavy chain variable region comprising a HCDR1 amino acid sequence selected from the group consisting of:
X₁X₂X₃MX₄ (SEQ ID NO: 556), wherein X₁ is A, R, T or S, wherein X₂ is Y or N, wherein X₃ is A or W, and wherein X₄ is S or N;
X₁YDIN (SEQ ID NO: 563), wherein X₁ is R or S; GYYX₁H, wherein X₁ is M or I;

(SEQ ID NO: 581)
AYAMS;
(SEQ ID NO: 582)
RYDIN;
and
(SEQ ID NO: 583)
SYWMS.

In various embodiments, the TREM-1 antigen binding protein comprises an antigen binding domain comprising a sequence having a heavy chain variable region comprising a HCDR2 amino acid sequence selected from the group consisting of:
X₁X₂X₃X₄X₅X₆ X₇X₈ X₉YYX₁₀X₁₁X₁₂VKG (SEQ ID NO: 559), wherein X₁ is T, E, or S, wherein X₂ is absent or is M, V, or I, wherein X₃ is S, R or K, wherein X₄ is G or Q, wherein X₅ is S, D or H, wherein X₆ is G, S L, or A, wherein X₇ is S, G, or R, wherein X₈ is T, S, P or E, wherein X₉ is T or I, wherein X₁₀ is A or V, wherein X₁₁ is D or E, and wherein X₁₂ is S or A;
X₁X₂NPX₃X₄GX₅X₆GX₇X₈ X₉X₁₀FX₁₁X₁₂ (SEQ ID NO: 564), wherein X₁ is W or R, wherein X₂ is M or L, wherein X₃ is N, Q, or K, wherein X₄ is S, A, or R, wherein X₅ is N, or Q, wherein X₆ is S, A, or T, wherein X₇ is S, Q, or Y, wherein X₈ is V or T, wherein X₉ is Q or K, wherein X₁₀ is K or N, wherein X₁₁ is R or Q, and wherein X₁₂ is G or D;

(SEQ ID NO: 584)
TSGSGSTTYYADSVKG;
(SEQ ID NO: 585)
WMNPNSGNSSVQKFRG;
(SEQ ID NO: 586)
NIKQDGSEEYYVDSVKG;
and
(SEQ ID NO: 841)
TSGSGTYYADSVKG.

In various embodiments, the TREM-1 antigen binding protein comprises an antigen binding domain comprising a sequence having a heavy chain variable region comprising a HCDR3 amino acid sequence selected from the group consisting of:
X₁X₂X₃X₄X₅X₆ X₇ F X₈YYX₉ (SEQ ID NO: 557), wherein X₁ is V, E, A or G, wherein X₂ is A, F, Y or G, wherein X₃ is G, S, Y or W, wherein X₄ is S or R, wherein X₅ is absent or is N, wherein X₆ is F, S, Y, or absent, wherein X₇ is L or F or absent, wherein X₈ is D or E, and wherein X₉ is Y, H or S;
X₁X₂X₃X₄ X₅X₆ X₇X₈ X₉X₁₀X₁₁X₁₂FX₁₃X₁₄ (SEQ ID NO: 565); wherein X₁ is G, L or R, wherein X₂ is G, I, or R, wherein X₃ is Y, R, I, G, or A, wherein X₄ is T, S, Y, or V, wherein X₅ is S or Y, wherein X₆ is S, A, I, or R, wherein X₇ is W, A, or S, wherein X₈ is absent or is S, wherein X₉ is absent or is F, W, or Y, wherein X₁₀ is R, S, H, K, or E, wherein X₁₁ is W, H, Y, or F, wherein X₁₂ is Y, V, A, or S, wherein X₁₃ is D or Q, and wherein X₁₄ is L, Y, I, or H;

(SEQ ID NO: 842)
VAGSNFLFDY;
(SEQ ID NO: 843)
GGYTSSWRWYFDL;
(SEQ ID NO: 844)
GGYTSSWSRWYFDL;
and
(SEQ ID NO: 845)
DYGDSFDY.

In various embodiments, provided herein is an isolated antigen binding protein, wherein the antigen binding protein:
a. is an antibody or antibody fragment;
b. binds to human TREM-1 having the amino acid sequence set forth in SEQ ID NO: 2;
c. comprises a light chain variable domain comprising:
   i. a light chain CDR1 comprising an amino acid sequence X₁ASQSX₂X₃X₄NLA (SEQ ID NO: 553), wherein X₁ is R or Q, wherein X₂ is V or I, wherein X₃ is N or S, and wherein X₄ is S, H, I, V or A;
   ii. a light chain CDR2 comprising an amino acid sequence GAX₁X₂RAT (SEQ ID NO: 554), wherein X₁ is S or Y, and wherein X₂ is T or I; and
   iii. a light chain CDR3 comprising an amino acid sequence QX₁X₂X₃X₄X₅X₆PX₇T (SEQ ID NO: 555); wherein X₁ is Q, H or E, wherein X₂ is F or Y, wherein X₃ is K, Y or I, wherein X₄ is N, T, L, I, or M; wherein X₅ is W, F, H or Y, wherein X₆ is absent or P; wherein X₇ is W, N, Y, H or L; and
d. comprises a heavy chain variable domain comprising:
   i. a heavy chain CDR1 comprising an amino acid sequence X₁X₂X₃MX₄ (SEQ ID NO: 556), wherein X₁ is A, R, T or S, wherein X₂ is Y or N, wherein X₃ is A or W, and wherein X₄ is S or N;
   ii. a heavy chain CDR2 comprising an amino acid sequence X₁X₂X₃X₄X₅X₆ X₇X₈ X₉YYX₁₀X₁₁X₁₂VKG (SEQ ID NO: 559), wherein X₁ is T, E, or S, wherein X₂ is absent or is M, V, or I, wherein X₃ is S, R or K, wherein X₄ is G or Q, wherein X₅ is S, D or H, wherein X₆ is G, S L, or A, wherein X₇ is S, G, or R, wherein X₈ is T, S, P or E, wherein X₉ is T or I, wherein X₁₀ is A or V, wherein X₁₁ is D or E, and wherein X₁₂ is S or A; and
   iii. a heavy chain CDR3 comprising an amino acid sequence X₁X₂X₃X₄X₅X₆ X₇ F X₈YYX₉ (SEQ ID NO: 557), wherein X₁ is V, E, A or G, wherein X₂ is A, F, Y or G, wherein X₃ is G, S, Y or W, wherein X₄ is S or R, wherein X₅ is absent or is N, wherein X₆ is F, S, Y, or absent, wherein X₇ is L or F or absent, wherein X₈ is D or E, and wherein X₉ is Y, H or S.

In various embodiments, the antigen binding protein comprises:
a. a light chain variable domain comprising:
i. a light chain CDR1 comprising an amino acid sequence RASQSVNSNLA (SEQ ID NO: 556);
ii. a light chain CDR2 comprising an amino acid sequence GASTRAT (SEQ ID NO: 573);
iii. a light chain CDR3 comprising an amino acid sequence QQFKNWPPT (SEQ ID NO: 576); and
b. a heavy chain variable domain comprising:
   i. a heavy chain CDR1 comprising an amino acid sequence AYAMS (SEQ ID NO: 581);
   ii. a heavy chain CDR2 comprising an amino acid sequence TSGSGSTTYYADSVKG (SEQ ID NO: 584); and
   iii. a heavy chain CDR3 comprising an amino acid sequence VAGSNFLFDY (SEQ ID NO: 842).

In various embodiments, the disclosure provides an isolated antigen binding protein, wherein the antigen binding protein:
a. is an antibody or antibody fragment;
b. binds to human TREM-1 having the amino acid sequence set forth in SEQ ID NO: 2;
c. comprises a light chain variable domain comprising:
   i. a light chain CDR1 comprising an amino acid sequence QASX₁DIX₂X₃X₄LN (SEQ ID NO: 558), wherein X₁ is R or Q, wherein X₂ is R, S, N or F, wherein X₃ is K or N, and wherein X₄ is H, Y or D;
   ii. a light chain CDR2 comprising an amino acid sequence X₁X₂X₃X₄LET (SEQ ID NO: 560), wherein X, is D, G or H, wherein X₂ is A, V or T, wherein X₃ is S, A or Y, and wherein X₄ is T or N;
   iii. a light chain CDR3 comprising an amino acid sequence QX₁YX₃X₄X₅PX₆T (SEQ ID NO: 561), wherein X₁ is Q or H, wherein X₂ is D, A or G, wherein X₃ is N or K; wherein X₄ is L or I, and wherein X₅ is I or L; and
d. comprises a heavy chain variable domain comprising:
   i. a heavy chain CDR1 comprising an amino acid sequence X₁YDIN (SEQ ID NO: 563), wherein X₁ is R or S;
   ii. a heavy chain CDR2 comprising an amino acid sequence X₁X₂NPX₃X₄GX₅X₆GX₇X₈ X₉X₁₀FX₁₁X₁₂ (SEQ ID NO: 564), wherein X₁ is W or R, wherein X₂ is M or L, wherein X₃ is N, Q, or K, wherein X₄ is S, A, or R, wherein X₅ is N, or Q, wherein X₆ is S, A, or T, wherein X₇ is S, Q, or Y, wherein X₈ is V or T, wherein X₉ is Q or K, wherein X₁₀ is K or N, wherein X₁₁ is R or Q, and wherein X₁₂ is G or D; and
   iii. a heavy chain CDR3 comprising an amino acid sequence X₁X₂X₃X₄ X₅X₆ X₇X₈ X₉X₁₀X₁₁X₁₂FX₁₃X₁₄ (SEQ ID NO: 565); wherein X₁ is G, L or R, wherein X₂ is G, I, or R, wherein X₃ is Y, R, I, G, or A, wherein X₄ is T, S, Y, or V, wherein X₅ is S or Y, wherein X₆ is S, A, I, or R, wherein X₇ is W, A, or S, wherein X₈ is absent or is S, wherein X₉ is absent or is F, W, or Y, and wherein X₁₀ is R, S, H, K, or E, wherein X₁₁ is W, H, Y, or F, wherein X₁₂ is Y, V, A, or S, wherein X₁₃ is D or Q, and wherein X₁₄ is L, Y, I, or H.

In various embodiments, the antigen binding protein comprises:
a. a light chain variable domain comprising:
   i. a light chain CDR1 comprising an amino acid sequence QASQDIRKHLN (SEQ ID NO: 567);
   ii. a light chain CDR2 comprising an amino acid sequence DASNLET (SEQ ID NO: 574); and
   iii. a light chain CDR3 comprising an amino acid sequence QHYDNLPIT (SEQ ID NO: 577); and
b. a heavy chain variable domain comprising:
   i. a heavy chain CDR1 comprising an amino acid sequence RYDIN (SEQ ID NO: 582);
   ii. a heavy chain CDR2 comprising an amino acid sequence WMNPNSGNSSVQKFRG (SEQ ID NO: 585); and
   iii. a heavy chain CDR3 comprising an amino acid sequence

(SEQ ID NO: 843)
GGYTSSWRWYFDL
or
(SEQ ID NO: 844)
GGYTSSWSRWYFDL.

In various embodiments, the disclosure provides a method for treating cardiovascular disease (e.g., atherosclerosis or myocardial infarction) comprising administering an antigen binding protein, wherein the antigen binding protein:
a. is an antibody or antibody fragment;
b. binds to human TrigGering Receptor Expressed on Myeloid cells 1 (TREM-1) having the amino acid sequence set forth in SEQ ID NO: 2;
c. comprises a set of CDR sequences selected from:
   i) SEQ ID NO: 10 (LCDR1), SEQ ID NO: 11 (LCDR2), SEQ ID NO: 12 (LCDR3), SEQ ID NO: 16 (HCDR1), SEQ ID NO: 17 (HCDR2) and SEQ ID NO: 18 (HCDR3);
   ii) SEQ ID NO: 30 (LCDR1), SEQ ID NO: 31 (LCDR2), SEQ ID NO: 32 (LCDR3), SEQ ID NO: 36 (HCDR1), SEQ ID NO: 37 (HCDR2) and SEQ ID NO: 38 (HCDR3);
   iii) SEQ ID NO: 50 (LCDR1), SEQ ID NO: 51 (LCDR2), SEQ ID NO: 52 (LCDR3), SEQ ID NO: 56 (HCDR1), SEQ ID NO: 57 (HCDR2) and SEQ ID NO: 58 (HCDR3);
   iv) SEQ ID NO: 70 (LCDR1), SEQ ID NO: 71 (LCDR2), SEQ ID NO: 72 (LCDR3), SEQ ID NO: 76 (HCDR1), SEQ ID NO: 77 (HCDR2) and SEQ ID NO: 78 (HCDR3);
   v) SEQ ID NO: 90 (LCDR1), SEQ ID NO: 91 (LCDR2), SEQ ID NO: 92 (LCDR3), SEQ ID NO: 96 (HCDR1), SEQ ID NO: 97 (HCDR2) and SEQ ID NO: 98 (HCDR3);
   vi) SEQ ID NO: 110 (LCDR1), SEQ ID NO: 111 (LCDR2), SEQ ID NO: 112 (LCDR3), SEQ ID NO: 116 (HCDR1), SEQ ID NO: 117 (HCDR2) and SEQ ID NO: 118 (HCDR3);
   vii) SEQ ID NO: 130 (LCDR1), SEQ ID NO: 131 (LCDR2), SEQ ID NO: 132 (LCDR3), SEQ ID NO: 136 (HCDR1), SEQ ID NO: 137 (HCDR2) and SEQ ID NO: 138 (HCDR3);
   viii) SEQ ID NO: 150 (LCDR1), SEQ ID NO: 151 (LCDR2), SEQ ID NO: 152 (LCDR3), SEQ ID NO: 156 (HCDR1), SEQ ID NO: 157 (HCDR2) and SEQ ID NO: 158 (HCDR3);
   ix) SEQ ID NO: 170 (LCDR1), SEQ ID NO: 171 (LCDR2), SEQ ID NO: 172 (LCDR3), SEQ ID NO: 176 (HCDR1), SEQ ID NO: 177 (HCDR2) and SEQ ID NO: 178 (HCDR3);
   x) SEQ ID NO: 190 (LCDR1), SEQ ID NO: 191 (LCDR2), SEQ ID NO: 192 (LCDR3), SEQ ID NO: 196 (HCDR1), SEQ ID NO: 197 (HCDR2) and SEQ ID NO: 198 (HCDR3);
   xi) SEQ ID NO: 210 (LCDR1), SEQ ID NO: 211 (LCDR2), SEQ ID NO: 212 (LCDR3), SEQ ID NO: 216 (HCDR1), SEQ ID NO: 217 (HCDR2) and SEQ ID NO: 218 (HCDR3);
   xii) SEQ ID NO: 230 (LCDR1), SEQ ID NO: 231 (LCDR2), SEQ ID NO: 232 (LCDR3), SEQ ID NO: 236 (HCDR1), SEQ ID NO: 237 (HCDR2) and SEQ ID NO: 238 (HCDR3);
   xiii) SEQ ID NO: 250 (LCDR1), SEQ ID NO: 251 (LCDR2), SEQ ID NO: 252 (LCDR3), SEQ ID NO: 256 (HCDR1), SEQ ID NO: 257 (HCDR2) and SEQ ID NO: 258 (HCDR3);
   ix) SEQ ID NO: 270 (LCDR1), SEQ ID NO: 271 (LCDR2), SEQ ID NO: 272 (LCDR3), SEQ ID NO: 276 (HCDR1), SEQ ID NO: 277 (HCDR2) and SEQ ID NO: 278 (HCDR3); and
   xv) SEQ ID NO: 544 (LCDR1), SEQ ID NO: 545 (LCDR2), SEQ ID NO: 546 (LCDR3), SEQ ID NO: 547 (HCDR1), SEQ ID NO: 548 (HCDR2) and SEQ ID NO: 549 (HCDR3).

In various embodiments, the anti-TREM-1 antigen-binding protein comprises a set of CDR sequences selected from:
i) SEQ ID NO: 10 (LCDR1), SEQ ID NO: 11 (LCDR2), SEQ ID NO: 12 (LCDR3), SEQ ID NO: 16 (HCDR1), SEQ ID NO: 17 (HCDR2) and SEQ ID NO: 18 (HCDR3);
ii) SEQ ID NO: 30 (LCDR1), SEQ ID NO: 31 (LCDR2), SEQ ID NO: 32 (LCDR3), SEQ ID NO: 36 (HCDR1), SEQ ID NO: 37 (HCDR2) and SEQ ID NO: 38 (HCDR3);
iii) SEQ ID NO: 50 (LCDR1), SEQ ID NO: 51 (LCDR2), SEQ ID NO: 52 (LCDR3), SEQ ID NO: 56 (HCDR1), SEQ ID NO: 57 (HCDR2) and SEQ ID NO: 58 (HCDR3);
iv) SEQ ID NO: 90 (LCDR1), SEQ ID NO: 91 (LCDR2), SEQ ID NO: 92 (LCDR3), SEQ ID NO: 96 (HCDR1), SEQ ID NO: 97 (HCDR2) and SEQ ID NO: 98 (HCDR3);
v) SEQ ID NO: 130 (LCDR1), SEQ ID NO: 131 (LCDR2), SEQ ID NO: 132 (LCDR3), SEQ ID NO: 136 (HCDR1), SEQ ID NO: 137 (HCDR2) and SEQ ID NO: 138 (HCDR3);
vi) SEQ ID NO: 150 (LCDR1), SEQ ID NO: 151 (LCDR2), SEQ ID NO: 152 (LCDR3), SEQ ID NO: 156 (HCDR1), SEQ ID NO: 157 (HCDR2) and SEQ ID NO: 158 (HCDR3); or
vii) SEQ ID NO: 270 (LCDR1), SEQ ID NO: 271 (LCDR2), SEQ ID NO: 272 (LCDR3), SEQ ID NO: 276 (HCDR1), SEQ ID NO: 277 (HCDR2) and SEQ ID NO: 278 (HCDR3).

In various embodiments, the anti-TREM-1 antigen binding protein comprises a set of CDR sequences selected from SEQ ID NO: 30 (LCDR1), SEQ ID NO: 31 (LCDR2), SEQ ID NO: 32 (LCDR3), SEQ ID NO: 36 (HCDR1), SEQ ID NO: 37 (HCDR2) and SEQ ID NO: 38 (HCDR3); or SEQ ID NO: 90 (LCDR1), SEQ ID NO: 91 (LCDR2), SEQ ID NO: 92 (LCDR3), SEQ ID NO: 96 (HCDR1), SEQ ID NO: 97 (HCDR2) and SEQ ID NO: 98 (HCDR3).

In various embodiments, the disclosure provides a method for treating cardiovascular disease (e.g., atherosclerosis or myocardial infarction) comprising administering an anti-TREM-1 antigen binding protein, wherein the anti-TREM-1 antigen-binding protein comprises:
a. a light chain variable domain comprising an amino acid sequence selected from the group consisting of:
   i. a sequence at least 80% identical to an amino acid sequence selected from SEQ ID NOS: 21, 41, 61, 81, 101, 121, 141, 161, 181, 201, 221, 241, 261, 281 and 539;
   ii. a sequence encoded by a polynucleotide sequence that is at least 80% identical to a nucleic acid sequence selected from SEQ ID NOS: 19, 39, 59, 79, 99, 119, 139, 159, 179, 199, 219, 239, 259, 279, and 537;
   iii. a sequence encoded by a polynucleotide that hybridizes under moderately stringent conditions to the complement of a polynucleotide consisting of a nucleic acid sequence selected from SEQ ID NOS: 19, 39, 59, 79, 99, 119, 139, 159, 179, 199, 219, 239, 259, 279 and 537; and
b. a heavy chain variable domain comprising an amino acid sequence selected from the group consisting of:
   i. a sequence that is at least 80% identical to an amino acid sequence selected from SEQ ID NOS: 22, 42, 62, 82, 102, 122, 142, 162, 182, 202, 222, 242, 262, 282, and 540;
   ii. a sequence encoded by a polynucleotide sequence that is at least 80% identical to an amino acid sequence selected from SEQ ID NOS: 20, 40, 60, 80, 100, 120, 140, 160, 180, 200, 220, 240, 260, 280, and 2184; iii. a sequence encoded by a polynucleotide that hybridizes under moderately stringent conditions to the complement of a polynucleotide consisting of a nucleic sequence selected from SEQ ID NOS: 20, 40, 60, 80, 100, 120, 140, 160, 180, 200, 220, 240, 260, 280, and 538.

In various embodiments, the anti-TREM-1 antigen-binding protein comprises:
a. a light chain variable domain comprising an amino acid sequence selected from the group consisting of:
   i. a sequence at least 80% identical to an amino acid sequence selected from SEQ ID NOS: 21, 41, 61, 101, 141, 161, and 281;
   ii. a sequence encoded by a polynucleotide sequence that is at least 80% identical to a nucleic acid sequence selected from SEQ ID NOS: 19, 39, 59, 99, 139, 159, and 279;
   iii. a sequence encoded by a polynucleotide that hybridizes under moderately stringent conditions to the complement of a polynucleotide consisting of a nucleic acid sequence selected from SEQ ID NOS: 19, 39, 59, 99, 139, 159, and 279; and
b. a heavy chain variable domain comprising an amino acid sequence selected from the group consisting of:
   i. a sequence that is at least 80% identical to an amino acid sequence selected from SEQ ID NOS: 22, 42, 62, 102, 142, 162, and 282;
   ii. a sequence encoded by a polynucleotide sequence that is at least 80% identical to an amino acid sequence selected from SEQ ID NOS: 20, 40, 60, 100, 140, 160, and 280;
   iii. a sequence encoded by a polynucleotide that hybridizes under moderately stringent conditions to the complement of a polynucleotide consisting of a nucleic sequence selected from SEQ ID NOS: 20, 40, 60, 100, 140, 160, and 280.

In various embodiments, the anti-TREM-1 antigen-binding protein comprises:
a. a light chain variable domain comprising an amino acid sequence selected from the group consisting of:
   i. a sequence at least 80% identical to an amino acid sequence selected from SEQ ID NOS: 41 and 101;
   ii. a sequence encoded by a polynucleotide sequence that is at least 80% identical to a nucleic acid sequence selected from SEQ ID NOS: 39 and 99;
   iii. a sequence encoded by a polynucleotide that hybridizes under moderately stringent conditions to the complement of a polynucleotide consisting of a nucleic acid sequence selected from SEQ ID NOS: 39 and 99; and
b. a heavy chain variable domain comprising an amino acid sequence selected from the group consisting of:
   i. a sequence that is at least 80% identical to an amino acid sequence selected from SEQ ID NOS: 42 and 102;
   ii. a sequence encoded by a polynucleotide sequence that is at least 80% identical to an amino acid sequence selected from SEQ ID NOS: 40 and 100;
   iii. a sequence encoded by a polynucleotide that hybridizes under moderately stringent conditions to the complement of a polynucleotide consisting of a nucleic sequence selected from SEQ ID NOS: 40 and 100.

In various embodiments, the anti-TREM-1 antigen-binding protein comprises: i) a light chain variable domain sequence set out in SEQ ID NO: 21 and a heavy chain variable domain sequence set out in SEQ ID NO: 22;
i) a light chain variable domain sequence set out in SEQ ID NO: 41 and a heavy chain variable domain sequence set out in SEQ ID NO: 42;
ii) a light chain variable domain sequence set out in SEQ ID NO: 61 and a heavy chain variable domain sequence set out in SEQ ID NO: 62;
iii) a light chain variable domain sequence set out in SEQ ID NO: 81 and a heavy chain variable domain sequence set out in SEQ ID NO: 82;
iv) a light chain variable domain sequence set out in SEQ ID NO: 101 and a heavy chain variable domain sequence set out in SEQ ID NO: 102;
v) a light chain variable domain sequence set out in SEQ ID NO: 121 and a heavy chain variable domain sequence set out in SEQ ID NO: 122;
vi) a light chain variable domain sequence set out in SEQ ID NO: 161 and a heavy chain variable domain sequence set out in SEQ ID NO: 162;
vii) a light chain variable domain sequence set out in SEQ ID NO: 181 and a heavy chain variable domain set out in SEQ ID NO: 182;
viii) a light chain variable domain sequence set out in SEQ ID NO: 201 and a heavy chain variable domain set out in SEQ ID NO: 202;
x) a light chain variable domain sequence set out in SEQ ID NO: 221 and a heavy chain variable domain sequence set out in SEQ ID NO: 222;
xi) a light chain variable domain set out in SEQ ID NO: 241 and a heavy chain variable domain set out in SEQ ID NO: 242;
xii) a light chain variable domain sequence set out in SEQ ID NO: 261 and a heavy chain variable domain sequence set out in SEQ ID NO: 262;
xiii) a light chain variable domain sequence set out in SEQ ID NO: 281 and a heavy chain variable domain sequence set out in SEQ ID NO: 282; and
xv) a light chain variable domain sequence set out in SEQ ID NO: 539 and a heavy chain variable domain sequence set out in SEQ ID NO: 540.

In various embodiments, the anti-TREM-1 antigen-binding protein comprises:
i) a light chain variable domain sequence set out in SEQ ID NO: 21 and a heavy chain variable domain sequence set out in SEQ ID NO: 22;
ii) a light chain variable domain sequence set out in SEQ ID NO: 41 and a heavy chain variable domain sequence set out in SEQ ID NO: 42;
iii) a light chain variable domain sequence set out in SEQ ID NO: 61 and a heavy chain variable domain sequence set out in SEQ ID NO: 62;
iv) a light chain variable domain sequence set out in SEQ ID NO: 101 and a heavy chain variable domain sequence set out in SEQ ID NO: 102;
v) a light chain variable domain sequence set out in SEQ ID NO: 141 and a heavy chain variable domain sequence set out in SEQ ID NO: 142;
vi) a light chain variable domain sequence set out in SEQ ID NO: 161 and a heavy chain variable domain sequence set out in SEQ ID NO: 162; or
vii) a light chain variable domain sequence set out in SEQ ID NO: 281 and a heavy chain variable domain set out in SEQ ID NO: 282.

In various embodiments, the anti-TREM-1 antigen-binding protein comprises: a light chain variable domain sequence set out in SEQ ID NO: 4 and a heavy chain variable domain sequence set out in SEQ ID NO: 42; or a light chain variable domain sequence set out in SEQ ID NO: 101 and a heavy chain variable domain sequence set out in SEQ ID NO: 102.

In various embodiments, the amino acid sequences can be 80%, 85%, 90%, 91%, 92%, 93%, 94%, 95%, 96%, 97%, 98% or 99% identical to SEQ ID NOS: 22, 42, 62, 82, 102. 122, 142, 162, 182, 202, 222, 242, 262, 282, and 540, and SEQ ID NOS: 21, 41, 61, 81, 101, 121, 141, 161, 181, 201, 221, 241, 261, 281, and 539.

In various embodiments, the antigen binding protein comprises an amino acid sequence at least 90% identical to a heavy chain variable region amino acid sequence selected from SEQ ID NOS: 22, 42, 62, 82, 102. 122, 142, 162, 182, 202, 222, 242, 262, 282, and 540.

In various embodiments, the antigen-binding protein comprises an amino acid sequence at least 90% identical to a light chain variable region amino acid sequence set forth in SEQ ID NOS: 21, 41, 61, 81, 101, 121, 141, 161, 181, 201, 221, 241, 261, 281, and 539.

In various embodiments, the antigen binding protein comprises an amino acid sequence at least 90% identical to a heavy chain variable region amino acid sequence selected from SEQ ID NOS: 22, 42, 62, 102, 142, 162, and 282. In various embodiments, the antigen-binding protein comprises an amino acid sequence at least 90% identical to a light chain variable region amino acid sequence selected from SEQ ID NO: 21, 41, 61, 101, 141, 161, and 281.

In various embodiments, the antigen binding protein comprises an amino acid sequence at least 90% identical to a heavy chain variable region amino acid sequence selected from SEQ ID NOS: 42 and 102. In various embodiments, the antigen-binding protein comprises an amino acid sequence at least 90% identical to a light chain variable region amino acid sequence selected from SEQ ID NO: 41 and 101.

In various embodiments, one or more heavy chain framework amino acids of the anti-antigen-binding protein are replaced with corresponding amino acid(s) from another human antibody amino acid sequence. In various embodiments, one or more light chain framework amino acids of the antigen-binding protein are replaced with corresponding amino acid(s) from another human antibody amino acid sequence.

In some instances, a sequence disclosed herein may contain an N-terminal signal sequence useful for recombinant production. Contemplated herein are sequences of anti-TREM-1 antibodies lacking the signal sequences. Exemplary signal sequences include MDMRVPAQLLGLLLLWLRGARC; MAWALLLLTLLTQGTGSWASYEL, and nucleic acids encoding such signal sequences.

In various embodiments, the anti-TREM-1 antigen binding protein further comprises a human light chain constant region attached to said light chain variable region.

In various embodiments, the heavy chain constant region is selected from heavy chain constant regions of an IgG, IgM, IgA, IgD, IgE, fragments thereof, combinations thereof, and modifications thereof in which one to ten heavy chain framework amino acids are replaced with corresponding amino acid(s) from another human antibody amino acid sequence.

In various embodiments, the anti-TREM-1 antigen-binding protein described herein inhibits binding of a TREM-1 ligand to TREM-1.

Also contemplated is an antigen binding protein that competes for binding to a human TREM-1 protein having the sequence of SEQ ID NO: 2 with an anti-TREM-1 antigen binding protein as described herein.

In various embodiments, the antigen-binding protein is selected from the group consisting of a human antibody, a humanized antibody, a chimeric antibody, a monoclonal antibody, a recombinant antibody, a Fab, a F(ab′)2, a Fab2, a monovalent IgG, an scFv, an scFv-Fc, an IgG1 antibody, an IgG2 antibody, an IgG3 antibody, and an IgG4 antibody. In various embodiments, the anti-TREM-1 antigen-binding protein is an IgG2 antibody. In various embodiments, the anti-TREM-1 antigen-binding protein is an IgG1 antibody. In various embodiments, the IgG1 antibody is an IGg1z or IgG1z-SEFL2 antibody. In various embodiments, the antigen-binding protein is a monovalent IgG.

In various embodiments, the antigen-binding protein is a human antibody.

Also provided is an isolated nucleic acid molecule comprising a nucleotide sequence that encodes the heavy chain of an anti-TREM-1 antigen binding protein as described herein, an isolated nucleic acid molecule comprising a nucleotide sequence that encodes the light chain of an anti-TREM-1 antigen binding protein as described herein, and an isolated nucleic acid molecule comprising a nucleotide sequence that encodes the heavy chain and light chain of an anti-TREM-1 antigen binding protein as described herein.

In various embodiments, the antigen binding protein comprises an amino acid sequence at least 90% identical to a heavy chain variable region polynucleotide sequence selected from SEQ ID NOS: 20, 40, 60, 80, 100, 120, 140, 160, 180, 200, 220, 240, 260, 280 and 538. In various embodiments, the antigen-binding protein comprises an amino acid sequence at least 90% identical to a light chain variable region polynucleotide sequence set forth in SEQ ID NO: 19, 39, 59, 79, 99, 119, 139, 159, 179, 199, 219, 239, 259, 279 and 537.

In various embodiments, the antigen binding protein comprises an amino acid sequence at least 90% identical to a heavy chain variable region polynucleotide sequence selected from SEQ ID NOS: 20, 40, 60, 100, 140, 160, and 280. In various embodiments, the antigen-binding protein comprises an amino acid sequence at least 90% identical to a light chain variable region polynucleotide sequence selected from SEQ ID NO: 19, 39, 59, 99, 139, 159, and 279.

In various embodiments, the antigen binding protein comprises an amino acid sequence at least 90% identical to a heavy chain variable region polynucleotide sequence selected from SEQ ID NOS: 40 and 100. In various embodiments, the antigen-binding protein comprises an amino acid sequence at least 90% identical to a light chain variable region polynucleotide sequence selected from SEQ ID NO: 39 and 99.

Further contemplated is an expression vector comprising the nucleic acid molecules of an anti-TREM-1 antigen binding protein heavy and/or light chain as described herein operably linked to an expression control sequence.

The disclosure provides a recombinant host cell comprising the nucleic acid molecule comprising a nucleotide sequence encoding the heavy chain of an anti-TREM-1 antigen binding protein or antibody as described herein; or the nucleic acid molecule encoding the light chain or an anti-TREM-1 antibody as described herein; or the nucleic acid molecule encoding a heavy chain and light chain nucleic acid molecule of an anti-TREM-1 antibody as described herein; or the vector comprising the nucleic acid molecules encoding the heavy and/or light chain of an anti-TREM-1 antibody as described herein. In various embodiments, the host cell is a mammalian cell. In various embodiments, the host cell is a CHO cell.

Further provided is a method of using the host cell to produce an antigen-binding protein, comprising culturing the host cell and recovering said antigen-binding protein, and an antigen-binding protein produced by the method.

Also provided is a sterile pharmaceutical composition comprising the anti-TREM-1 antigen binding protein as described herein and a pharmaceutically acceptable carrier.

In various embodiments, the antigen binding protein further comprises a human heavy chain constant region attached to said heavy chain variable region of the antigen binding protein. In various embodiments, the antigen binding protein further comprises a human light chain constant region attached to said light chain variable region of the antibody.

In various embodiments, the antigen-binding moiety is selected from the group consisting of a human antibody, a humanized antibody, a chimeric antibody, a monoclonal antibody, a recombinant antibody, a Fab, a F(ab′)2, a Fab2, a monovalent IgG, an scFv, an scFv-Fc, an IgG1 antibody, an IgG2 antibody, an IgG3 antibody, and an IgG4 antibody. In various embodiments, the antigen-binding moiety is an IgG. In various embodiments, the antigen-binding moiety is an IgG2 antibody. In various embodiments, the antigen-binding moiety is an IgG1 antibody. In various embodiments of the antigen binding protein, the antibody is an IgG1z or IgG1z-SEFL2 antibody. In various embodiments, the antigen-binding moiety is a monovalent IgG. In various embodiments, the heavy chain constant region of the antigen-binding moiety is selected from heavy chain constant regions of an IgG, IgM, IgA, IgD, IgE, fragments thereof, combinations thereof, and modifications thereof in which one to ten heavy chain framework amino acids are replaced with corresponding amino acid(s) from another human antibody constant region.

In various embodiments, the antigen binding protein has the heavy and light chain pairs as set out in Table 7-8, Table 11 and Table 12.

Nucleic acid sequences of TREM-1 variant antibody heavy chain variable regions are set out in SEQ ID NOS: 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392,394, 396, 398, 400, 402, 404, 406, and 408, and also set out in SEQ ID NOS: 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, and 536; and TREM-1 variant antibody light chain variable regions nucleotide sequences are set out in SEQ ID NO: 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, and also set out in SEQ ID NO: 588, 590, 592, 594, 596, 598, 600, 602, 604, 606, 608, 610, 612, 614, 61, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, and 712.

In various embodiments, one or more heavy chain framework amino acids of the antigen-binding protein are replaced with corresponding amino acid(s) from another human antibody amino acid sequence. In various embodiments, one or more light chain framework amino acids of the antigen-binding protein are replaced with corresponding amino acid(s) from another human antibody amino acid sequence.

In various embodiments, the anti-TREM-1 antigen binding protein further comprises a human light chain constant region attached to said light chain variable region.

In various embodiments, provided herein are variants of the TREM-1 antibody heavy chain and/or light chain variable regions. TREM-1 antibody heavy chain variable region variant sequences are set out in SEQ ID NO: 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, Table 7-8, Table 11, and Table 12.

TREM-1 antibody light chain variable region variant sequences are set out in SEQ ID NOS: 715-777, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, Table 7-8, Table 11, and Table 12.

In various embodiments, the antigen-binding protein is a human antibody.

Also provided is an isolated nucleic acid molecule comprising a nucleotide sequence that encodes the heavy chain region of the antigen binding protein of as described herein, an isolated nucleic acid molecule comprising a nucleotide sequence that encodes the light chain region of the antigen binding protein of as described herein.

The disclosure provides an expression vector or vectors comprising the nucleic acid molecule or molecules encoding an antigen binding protein as described herein operably linked to an expression control sequence.

Also contemplated is a recombinant host cell comprising the nucleic acid molecule comprising an antigen binding protein as described herein, or the vector comprising said nucleic acid. In various embodiments, the host cell is a mammalian cell. In various embodiments, the host cell is a CHO cell. The disclosure provides a method of using the host cell described herein to produce an antigen binding protein, comprising culturing the host cell and recovering said antibody, and provides an antigen binding protein produced by the method.

The disclosure contemplates methods of treatment comprising administering a pharmaceutical composition comprising an anti-TREM-1 antigen binding protein or antigen binding fragment described herein.

In a related aspect, the disclosure provides a method of treating a cardiovascular disease (e.g., atherosclerosis or myocardial infarction) in a subject in a need thereof comprising administering an anti-TREM-1 antigen-binding protein as described herein or a composition comprising an anti-TREM-1 antigen binding protein as described herein.

Also contemplated is a composition comprising an anti-TREM-1 antigen binding protein or antigen-binding fragment as described herein for use in treating a cardiovascular disease (e.g., atherosclerosis or myocardial infarction). In various embodiments, the disclosure provides use of composition comprising an anti-TREM-1 antigen binding protein or antigen-binding fragment as described herein in preparation of a medicament for treating a cardiovascular disease (e.g., atherosclerosis or myocardial infarction).

In various embodiments, the cardiovascular disease is selected from the group consisting of myocardial infarction, non-alcoholic fatty liver disease (NAFLD), non-alcoholic steatohepatitis (NASH), heart failure, stroke (e.g., ischemic, hemorrhagic), atherosclerosis, coronary artery disease, peripheral vascular disease (e.g. peripheral artery disease), vulnerable plaque, acute coronary syndrome, cerebrovascular disease, cerebrovascular atherosclerosis, and obesity. In various embodiments the cardiovascular disease is myocardial infarction. In various embodiments, the cardiovascular disease is stroke (ischemic and/or hemorrhagic). In various embodiments, the cardiovascular disease is atherosclerosis. In various embodiments, the cardiovascular disease is NAFLD or NASH. In various embodiments, the cardiovascular disease is heart failure. In various embodiments, the cardiovascular disease is coronary artery disease. In various embodiments, the cardiovascular disease is peripheral vascular disease. In various embodiments, the cardiovascular disease is peripheral artery disease. In various embodiments, the cardiovascular disease is vulnerable plaque. In various embodiments, the cardiovascular disease is acute coronary syndrome. In various embodiments, the cardiovascular disease is cerebrovascular disease. In various embodiments, the cardiovascular disease is cerebrovascular atherosclerosis. In various embodiments, the cardiovascular disease is obesity.

In various embodiments, the treatment is administered intravenously or subcutaneously. In various embodiments, the treatment is administered once weekly, once every two weeks, once every three weeks, once every 4 weeks, once monthly, once every 3 months, or once every six months, or once yearly.

In various embodiments, the TREM-1 is human TREM-1 set out in SEQ ID NO: 2.

In various embodiments, the administration reduces one or more symptoms of cardiovascular disease selected from the group consisting of inflammatory cell migration to the site of injury, infiltration of myeloid cells into cardiac tissue, inflammatory cytokines in the microenvironment, tissue damage, reduction in foam cell formation, reduction in necrotic core size, reduction in scar formation, reduction in endothelial cell dysfunction, and/or reduction in thrombus formation.

In various embodiments, the methods comprise administering one or two additional therapeutic agents. In various embodiments, the additional therapeutic agents are selected from corticosteroids, NSAIDs, analgesics, immunosuppressive agents, anti-inflammatory agents, TNFα inhibitors, IL-12/IL-23 inhibitors, IL-17 and IFN-γ. Therapeutic agents (apart from the antigen binding protein), useful in cardiovascular disease include, but are not limited to, at least one cholesterol-lowering (serum and/or total body cholesterol) agent or an agent. In some embodiments, the agent increases the expression of LDLR, have been observed to increase serum HDL levels, lower LDL levels or lower triglyceride levels. Exemplary agents include, but are not limited to, statins (atorvastatin, cerivastatin, fluvastatin, lovastatin, mevastatin, pitavastatin, pravastatin, rosuvastatin, simvastatin), PCSK9 inhibitors (Repatha®, Praluent®, Leqvio®) nicotinic acid (Niacin) (NIACOR®, NIASPAN® (slow release niacin), SLO-NIACIN® (slow release niacin), Fibric acid (LOPIDO (gemfibrozil), TRICOR® (fenofibrate), Bile acid sequestrants (QUESTRAN® (cholestyramine), colesevelam (WELCHOL®), COLESTID® (colestipol), Cholesterol absorption inhibitors (Zetia (ezetimibe)), combining nicotinic acid with statin (ADVICOR® (LOVASTATIN and NIASPAN®). Combining a statin with an absorption inhibitor (VYTORIN® (ZOCOR® and ZETIA®) and/or lipid modifying agents like PCSK9 inhibitors. In some embodiments, the antigen binding protein is combined with PPAR gamma agonists, PPAR alpha/gamma agonists, squalene synthase inhibitors, cholesterylester transfer protein (CETP) inhibitors, anti-hypertensives, anti-thrombotics (aspirin) anti-diabetic agents (such as sulphonyl ureas, insulin, GLP-1 analogs, DDPIV inhibitors, SGL2 inhibitors), ApoB modulators, MTP inhibitors, Corlanor® (ivabradine) I(f) current Inhibitor, omecamtiv mecarbil cardiac myosin activator, OLPASIRAN (AMG890) (siRNA) that lowers lipoprotein(a), AMG 594 cardiac troponin activator, and/or arteriosclerosis obliterans treatments. Also contemplated is combination treatment with AMG 609, a small interfering RNA (siRNA) that targets a variant of patatin like phospholipase domain containing 3 (PNPLA3 I148M). The antigen binding protein may also be combined with other anti-inflammatory agents.

In various embodiments, the additional therapeutic agents are selected from the group consisting of one or more cholesterol-lowering agent, an agent that increases the expression of LDLR, statins (atorvastatin, cerivastatin, fluvastatin, lovastatin, mevastatin, pitavastatin, pravastatin, rosuvastatin, simvastatin), PCSK9 inhibitors (Repatha®, Praluent®, Leqvio®), nicotinic acid (Niacin) (NIACOR®, NIASPAN® (slow release niacin), SLO-NIACIN® (slow release niacin), Fibric acid (LOPID® (gemfibrozil), TRICOR® (fenofibrate), Bile acid sequestrants (QUESTRAN® (cholestyramine), colesevelam (WELCHOL®), COLESTID® (colestipol), Cholesterol absorption inhibitors (Zetia (ezetimibe)), combination of nicotinic acid with statin (ADVICOR® (LOVASTATIN and NIASPAN®), combination of a statin with an absorption inhibitor (VYTORIN (ZOCOR® and ZETIA®) and/or lipid modifying agents like PCSK9 inhibitors, PPAR gamma agonists, PPAR alpha/gamma agonists, squalene synthase inhibitors, cholesterylester transfer protein (CETP) inhibitors, anti-hypertensives, anti-thrombotics (aspirin) anti-diabetic agents (such as sulphonyl ureas, insulin, GLP-1 analogs, GIPR antagonists, DDPIV inhibitors, SGL2 inhibitors), ApoB modulators, MTP inhibitors, Corlanor® (ivabradine) I(f) current Inhibitor, omecamtiv mecarbil cardiac myosin activator, OLPASIRAN (AMG890) (siRNA) that lowers lipoprotein(a), AMG 594 cardiac troponin activator, AMG 609, AMG 171 (Growth Differential Factor 15 (GDF15) analog), AMG 133 (gastric inhibitory polypeptide receptor (GIPR) antagonist and glucagon-like peptide 1 (GLP-1) receptor agonist), and/or arteriosclerosis obliterans treatments.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawing and/or claims.

### BRIEF DESCRIPTION OF THE DRAWINGS

FIGS. 1A-1D show inhibition of ligand (PGLYRP1/PGN) mediated TREM-1 signaling by anti-human TREM-1 antibodies in human (FIGS. 1C-1D) or cyno (FIGS. 1A-1B) PBMCs as measured by TNF a release.

FIG. 2 shows inhibition by anti-human TREM-1 Fabs of ligand (PGLYRP1/PGN) mediated signaling in a cell line overexpressing human TREM-1/DAP12 as measured by phosphorylation of spleen tyrosine kinase (pSYK).

FIG. 3 shows the levels of PGLYRP1 in wild type and TREM-1 knockout mice after LPS injection.

### DETAILED DESCRIPTION

The present disclosure provides TREM-1 antibodies that block binding of TREM-1 ligands to the receptor. Antibodies in accordance with this disclosure show targeting to immune cells and modulation of cellular activity and cytokine response useful to treat cardiovascular diseases, such as atherosclerosis, stroke or myocardial infarction.

### Definitions

The term “polypeptide binding agent” or “antigen binding protein” refers to a polypeptide that is capable of specifically binding an antigen, e.g. a target or its signaling partner, or that is capable of binding an antigen with a measurable binding affinity. Examples of polypeptide binding agents include antibodies, peptibodies, polypeptides and peptides, optionally conjugated to other peptide moieties or non-peptidic moieties. Antigens to which a polypeptide binding agent may bind include any proteinaceous or non-proteinaceous molecule that is capable of eliciting an antibody response, or that is capable of binding to a polypeptide binding agent with detectable binding affinity greater than non-specific binding. The antigen to which a modulating polypeptide binding agent binds may include a target, a signaling partner of a target, and/or a complex comprising the target and its signaling partner.

The term “antibody” is used in the broadest sense and includes fully assembled antibodies, tetrameric antibodies, monoclonal antibodies, polyclonal antibodies, multispecific antibodies (e.g., bispecific antibodies), antibody fragments that can bind an antigen (e.g., Fab′, F′(ab)2, Fv, single chain antibodies, diabodies), and recombinant peptides comprising the forgoing as long as they exhibit the desired biological activity. An “immunoglobulin” or “tetrameric antibody” is a tetrameric glycoprotein that consists of two heavy chains and two light chains, each comprising a variable region and a constant region. Antigen-binding portions may be produced by recombinant DNA techniques or by enzymatic or chemical cleavage of intact antibodies. Antibody fragments or antigen-binding portions include, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, a camelized antibody, a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

The term “monovalent IgG” as used herein refers to an IgG in which a single antigen-binding fragment (Fab) is fused to a complete constant domain fragment (Fc) engineered to heterodimerize through mutations in the C_{H}3 domain within the Fc. A monovalent IgG is also known as a “one armed” antibody.

“Monoclonal antibody” refers to an antibody obtained from a population of substantially homogeneous antibodies, i.e., the individual antibodies comprising the population are identical except for possible naturally occurring mutations that may be present in minor amounts.

“Antibody variant” as used herein refers to an antibody polypeptide sequence that contains at least one amino acid substitution, deletion, or insertion in the variable region of the natural antibody variable region domains. Variants may be substantially homologous or substantially identical to the unmodified antibody.

An “isolated” antibody is one that has been identified and separated and recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain, or HPLC methods. Isolated antibody includes the antibody in situ within recombinant cells since at least one component of the antibody's natural environment will not be present. Ordinarily, however, isolated antibody will be prepared by at least one purification step.

“Heavy chain variable region” as used herein refers to the region of the antibody molecule comprising at least one complementarity determining region (CDR) of said antibody heavy chain variable domain. The heavy chain variable region may contain one, two, or three CDR of said antibody heavy chain.

“Light chain variable region” as used herein refers to the region of an antibody molecule, comprising at least one complementarity determining region (CDR) of said antibody light chain variable domain. The light chain variable region may contain one, two, or three CDR of said antibody light chain, which may be either a kappa or lambda light chain depending on the antibody.

As used herein, an antibody that “specifically binds” is “antigen specific”, is “specific for” antigen target or is “immunoreactive” with an antigen refers to an antibody or polypeptide binding agent of the invention that binds an antigen with greater affinity than other antigens of similar sequence. In one aspect, the antigen binding protein of the invention, or fragments, variants, or derivatives thereof, will bind with a greater affinity to human antigen as compared to its binding affinity to similar antigens of other, i.e., non-human, species, but polypeptide binding agents that recognize and bind orthologs of the target are within the scope of the invention.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous.

The term “derivative” when used in connection with polypeptide binding agents and polypeptides of the invention refers to polypeptides chemically modified by such techniques as ubiquitination, conjugation to therapeutic or diagnostic agents, labeling (e.g., with radionuclides or various enzymes), covalent polymer attachment such as PEGylation (derivatization with polyethylene glycol) and insertion or substitution by chemical synthesis of amino acids such as ornithine, which do not normally occur in human proteins. Derivatives retain the binding properties of underivatized molecules of the invention.

A “linker,” as used herein, refers to a peptide that links two polypeptides. A linker can be from 1-80 amino acids in length. In some embodiments, a linker can be 2-40, 3-40, 3-30, or 3-20 amino acids long. In some embodiments, a linker can be a peptide of 18, 17, 16, 15, 14, 13, 12, 11, 10, 9, 8, 7, 6, 5, 4 or 3 amino acids long. In other embodiments, a linker can be 3-25, 3-18, 5-20, 6-18, or 10-20 amino acids long. In other embodiments, a linker can be about, 2,3,4,5, 6, 7, 8, 9, 10, 11, 12, 13, 14, 15, 16, 17, 18, 19, 20, 21, 22, 23, 24, 25, 26, 27, 28, 29, or 30 amino acids long. In many cases, linkers lack free cysteine residues (i.e. and are therefore not involved in disulfide bonds) and also do not contain N-glycosylation sites (that is, Asn-Xxx-Ser/Thr, where X can be any amino acid except proline). In certain embodiments, peptide having the sequence G3SG2 or G4S is a linker between an anti-TREM-1 antigen binding protein and an IL-10 mutein. Examples of other suitable linkers include G2, G3, G3S, G3P, G3Q, and G5, among many others. Each capital letter in the foregoing linkers refers to the conventional one-letter code for an amino acid and each number refers to the number of tandem repeats of the amino acid in the linker. For example, “G3SG2” refers to a linker having the sequence Gly-Gly-Gly-Ser-Gly-Gly. “G4S” refers to a linker having the sequence Gly-Gly-Gly-Gly-Ser.

A “therapeutically effective amount” of a drug used to treat a disease is an amount that can reduce the severity of a disease, reduce the severity of one or more symptoms associated with the disease or its treatment, or delay the onset of more serious symptoms or a more serious disease that can occur with some frequency following the treated condition.

“Treatment” of any disease mentioned herein encompasses an alleviation of at least one symptom of the disease, a reduction in the severity of the disease, or the delay or prevention of disease progression to more serious symptoms that may, in some cases, accompany the disease or lead to at least one other disease. Treatment need not mean that the disease is totally cured. A useful therapeutic agent needs only to reduce the severity of a disease, reduce the severity of one or more symptoms associated with the disease or its treatment, or delay the onset of more serious symptoms or a more serious disease that can occur with some frequency following the treated condition.

“Subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

“Myeloid cell” as used herein refers to subgroup of immune cells derived from blood progenitor cells of the myeloid lineage, and include granulocytes, monocytes, macrophages, dendritic cells (DCs). Myeloid cells serve an important function in protective immunity, often having phagocytic and antigen presenting cell (APC) functions. See e.g., de Kleer et al., Front. Immunol., 5:423, 2014.

### Antigen Binding Proteins and Antibodies

Immunoglobulin variable domains exhibit the same general structure of relatively conserved framework regions (FR) joined by three hypervariable regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk, J. Mol. Biol. 196:901-917, 1987; Chothia et al., Nature 342:878-883, 1989.

The hypervariable region of an antibody refers to the CDR amino acid residues of an antibody which are responsible for antigen-binding. The hypervariable region comprises amino acid residues from a CDR [e.g., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5th Ed. Public Health Service, National Institutes of Health, Bethesda, Md. (1991)] and/or those residues from a hypervariable loop (e.g., residues 26-32 (L1), 50-52 (L2) and 91-96 (L3) in the light chain variable domain and 26-32 (H1), 53-55 (H2) and 96-101 (H3) in the heavy chain variable domain as described by [Chothia et al., J. Mol.Biol. 196:901-917 (1987)]. CDRs have also been identified and numbered according to ImMunoGenTics (IMGT) numbering (Lefranc, M.-P., The Immunologist, 7, 132-136 (1999); Lefranc, M.-P. et al., Dev. Comp. Immunol., 27, 55-77 (2003), which describes the CDR locations in the light and heavy chain variable domains as follows: CDR1, approximately residues 27 to 38; CDR2, approximately residues 56 to 65; and, CDR3, approximately residues 105 to 116 (germline) or residues 105 to 117 (rearranged). In one embodiment, it is contemplated that the CDRs are located at approximately residues 26-31 (L1), 49-51 (L2) and 88-98 (L3) in the light chain variable domain and approximately residues 26-33 (H1), 51-58 (H2) and 97-110 (H3) in the heavy chain variable domain of an antibody heavy or light chain of approximately similar length to those disclosed herein. However, one of skill in the art understands that the actual location of the CDR residues may vary from the projected residues described above when the sequence of the particular antibody is identified. CDRs that are assigned with sequence identifiers disclosed herein are defined following Kabat methodology (Kabat and Wu, 1991) and are numbered using Amgen Reference numbering. Amgen Reference numbering is a structurally based numbering system built upon the Honegger and Plückthun numbering system for antibody variable regions described in Honegger and Pluckthun (J Mol Biol. 309(3):657-70, 2001). In various embodiments, an engineered IgG1 antibody is contemplated. In various embodiment, the antibody is an IgG1z or IgG1z-SEFL2 antibody as described herein.

Additionally, CDRs may be defined according to the accumulation of both Kabat and Chothia, AbM, Contact, North, Martin, and/or conformational definitions or any method of CDR determination well known in the art. AbM definition of CDRs is a compromise between Kabat and Chothia and uses Oxford Molecular's AbM antibody modeling software (Accelrys®). The “contact” definition of CDRs is based on observed antigen contacts, set forth in MacCallum et al., 1996, J. Mol. Biol., 262:732-745. The “conformational” definition of CDRs is based on residues that make enthalpic contributions to antigen binding (see, e.g., Makabe et al., 2008, J. Biol. Chem., 283:1 156-1 166). North has identified canonical CDR conformations using a different preferred set of CDR definitions (North et al., 2011, J. Mol. Biol. 406: 228-256). In another approach, referred to herein as the “conformational definition” of CDRs, the positions of the CDRs may be identified as the residues that make enthalpic contributions to antigen binding (Makabe et al., 2008, J Biol. Chem. 283:1 156-1 166). Martin definition (also called enhanced Chothia definition) combines the Kabat and Chothia definitions and differs from them only in the heavy chain, where CDR-H1 includes all residues of Kabat and Chothia while CDR-H2 is seven residues shorter than that defined by Kabat (Martin, Bioinformatics tools for antibody engineering. Handbook of Therapeutic Antibodies. Weinheim: Wiley-VCH Verlag GmbH; (2008). p. 95-117; see also the database maintained by the Institute of Structural and Molecular Biology at the University College London, http://www.bioinf.org.uk/abs/#cdrid). Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. For example, “combined” CDRs may also be used. As used herein, a CDR may refer to CDRs defined by any approach known in the art, including combinations of approaches. The methods used herein may utilize CDRs defined according to any of these approaches. For any given embodiment containing more than one CDR, the CDRs (or other residue of the antibody) may be defined in accordance with any of Kabat, Chothia, North, AbM, Contact, IMGT, Martin, combined Kabat and Chothia, and/or conformational definitions.

For example, the following Table shows several commonly used definitions of CDRs (for convenience, residue numbering according to Kabat numbering instead of Amgen reference numbering)

| Loop | Kabat | AbM | Chothia¹ | Contact² | IMGT |
|---|---|---|---|---|---|
| L1 | L24-L34 | L24-L34 | L26-L32 | L30-L36 | L27-L32 |
| L2 | L50-L56 | L50-L56 | L50-L52 | L46-L55 | L50-L51 |
| L3 | L89-L97 | L89-L97 | L91-L96 | L89-L96 | L89-L97 |
| H1 | H31-H35B | H26-H35B | H26-H32 . . . 34 | H30-H35B | H26-H35B |
| | (Kabat Numbering)³ | | | | |
| H1 | H31-H35 | H26-H35 | H26-H32 | H30-H35 | H26-H33 |
| | (Chothia Numbering) | | | | |
| H2 | H50-H65 | H50-H58 | H52-H56 | H47-H58 | H51-H56 |
| H3 | H95-H102 | H95-H102 | H96-H101 | H93-H101 | H93-H102 |

¹Some of these definitions (particularly for Chothia loops) vary depending on the individual publication examined. Some papers describe Chothia CDRs as: CDR-L1: L24-34; CDR-L2: L50-56; CDR-L3: L89-97; CDR-H1: H26-32; CDR-H2: H52-56; CDR-H3: H95-102.
²Any of the numbering schemes can be used for these CDR definitions, except the contact definition uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)

Framework region (or FR) residues are those variable domain residues other than the hypervariable region residues.

As described below, antibodies, including monoclonal, human, humanized, and other antibodies described herein, contemplated herein are typically generated recombinantly or through other methods of manipulating the genetic code in vitro or in vivo, and are therefore not necessarily reflective of a particular antibody that is found in nature.

### Anti-TREM-1 Antigen Binding Proteins

Previous studies to characterize TREM-1 signaling utilized agonistic antibodies of TREM-1 to mimic ligand activation of the receptor (Tessarz et al., Immunol Lett 116(2):111-6, 2008; Vandestienne et al., J Clin Invest 131(2):e142468, 2021). Decoy peptides have also been attempted to modulate TREM-1 activity (see e.g., International Patent Publication No. WO2014037565A).

The present disclosure encompasses use of amino acid molecules encoding target specific antibodies. The anti-TREM-1 antigen binding proteins, described herein, differentially modulate interaction of human TREM-1 and its ligands.

In some embodiments, an antigen binding protein is provided that comprises a polypeptide having an amino acid sequence at least about 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more identical to the heavy chain variable region set out in SEQ ID NOs: 22, 42, 62, 82, 102, 122, 142, 162, 182, 202, 222, 242, 262, 282, and 540 and/or an amino acid sequence an amino acid sequence at least about 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more identical to the light chain variable region set out in SEQ ID NOs: 21, 41, 61, 81, 101, 121, 141, 161, 181, 201, 221, 241, 261, 281 and 539, the antibody further comprising at least one, two, three, four, five or all of HCDR1, HCDR2, HCDR3, LCDR1, LCDR2 or LCDR3. In some embodiments, the amino acid sequence with percentage identity to the light chain variable region may comprise one, two or three of the light chain CDRs. In other embodiments, the amino acid sequence with percentage identity to the heavy chain variable region may comprise one, two, or three of the heavy chain CDRs.

In another embodiment, an antigen binding protein is provided that comprises a polypeptide having an amino acid sequence at least about 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more identical to all three HCDRs in the heavy chain variable region of an antibody sequence set out above or the CDRs: heavy chain CDR1 sequence set out in SEQ ID NO: 16, 36, 56, 76, 96, 116, 136, 156, 176, 196, 216, 236, 256, 276, and 550; heavy chain CDR2 sequence set out in SEQ ID NO: 17, 37, 57, 77, 97, 117, 137, 157, 177, 197, 217, 237, 257, 277, and 551; and heavy chain CDR3 sequence set out in SEQ ID NO: 18, 38, 58, 78, 98, 118, 138, 158, 178, 198, 218, 238, 258, 278, and 552.

In a related embodiment, an antigen binding protein is provided that comprises a polypeptide having an amino acid sequence at least about 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more identical to the all three LCDRs in the light chain variable region of an antibody sequence set out above or the CDRs: light chain CDR1 sequence set out in SEQ ID NO: 10, 30, 50, 70, 90, 110, 130, 150, 170, 190, 210, 230, 250, 270, and 544; light chain CDR2 sequence set out in SEQ ID NOS: 11, 31, 51, 71, 91, 111, 131, 151, 171, 191, 211, 231, 251, 271, and 545; light chain CDR3 sequence set out in SEQ ID NO: 12, 32, 52, 72, 92, 112, 132, 152, 172, 192, 212, 232, 252, 272, and 546.

It is contemplated that the antibodies of the disclosure may have one, or two or more amino acid substitutions in the CDR regions of the antibody, e.g., non-conservative or conservative substitutions. Also contemplated are consensus sequences of the TREM-1 antibody heavy and light chain CDRs and/or variable region sequences disclosed herein. For example, TREM-1 antibodies can comprise the following sequences. In various embodiments, the TREM-1 antibody comprises an antigen binding domain comprising a sequence having a light chain variable region comprising a LCDR1 amino acid sequence selected from the group consisting of: X₁ASQSX₂X₃X₄NLA (SEQ ID NO: 553), wherein X₁ is R or Q, wherein X₂ is V or I, wherein X₃ is N or S, and wherein X₄ is S, H, I, V or A; QASX₁DIX₂X₃X₄LN (SEQ ID NO: 558), wherein X₁ is R or Q, wherein X₂ is R, S, N or F, wherein X₃ is K or N, and wherein X₄ is H, Y or D; RASQSVNSNLA (SEQ ID NO: 566); QASQDIRKHLN (SEQ ID NO: 567); RASQDISSNLN (SEQ ID NO: 568); QASQDIHLN (SEQ ID NO: 569); RASQGIRKWLA (SEQ ID NO: 570) or RASQSVNSNLA (SEQ ID NO: 571) and SGDKLGERVS (SEQ ID NO: 572).

In various embodiments, the TREM-1 antibody comprises an antigen binding domain comprising a sequence having a light chain variable region comprising a LCDR2 amino acid sequence selected from the group consisting of GAX₁X₂RAT (SEQ ID NO: 554), wherein X₁ is S or Y, and wherein X₂ is T or I; an amino acid sequence X₁X₂X₃X₄LET (SEQ ID NO: 560), wherein X₁ is D, G or H, wherein X₂ is A, V or T, wherein X₃ is S, A or Y, and wherein X₄ is T or N; GASTRAT (SEQ ID NO: 573); DASNLET (SEQ ID NO: 574); and AASRLQS (SEQ ID NO: 575).

In various embodiments, the TREM-1 antibody comprises an antigen binding domain comprising a sequence having a light chain variable region comprising a LCDR3 amino acid sequence selected from the group consisting of QX₁X₂X₃X₄X₅X₆PX₇T (SEQ ID NO: 555); wherein X₁ is Q, H or E, wherein X₂ is F or Y, wherein X₃ is K, Y or I, wherein X₄ is N, T, L, I, or M; wherein X₅ is W, F, H or Y, wherein X₆ is absent or P; wherein X₇ is W, N, Y, H or L; QX₁YX₃X₄X₅PX₆T (SEQ ID NO: 561), wherein X₁ is Q or H, wherein X₂ is D, A or G, wherein X₃ is N or K; wherein X₄ is L or I, and wherein X₅ is I or L; QQFKNWPPT (SEQ ID NO: 576); QHYDNLPIT (SEQ ID NO: 577); LQAHGFPWT (SEQ ID NO: 578); QQYDNLPLT (SEQ ID NO: 579) and QFWPPWT (SEQ ID NO: 580).

In various embodiments, the TREM-1 antibody comprises an antigen binding domain comprising a sequence having a heavy chain variable region comprising a HCDR1 amino acid sequence selected from the group consisting of X₁X₂X₃MX₄ (SEQ ID NO: 556), wherein X₁ is A, R, T or S, wherein X₂ is Y or N, wherein X₃ is A or W, and wherein X₄ is S or N; a sequence X₁YDIN (SEQ ID NO: 563), wherein X₁ is R or S; GYYX₁H, wherein X₁ is M or I; AYAMS (SEQ ID NO: 581); RYDIN (SEQ ID NO: 582); and SYWMS (SEQ ID NO: 583).

In various embodiments, the TREM-1 antibody comprises an antigen binding domain comprising a sequence having a heavy chain variable region comprising a HCDR2 amino acid sequence selected from the group consisting of X₁X₂X₃X₄X₅X₆ X₇ Xₐ X₉YYX₁₀X₁₁X₁₂VKG (SEQ ID NO: 559), wherein X₁ is T, E, or S, wherein X₂ is absent or is M, V, or I, wherein X₃ is S, R or K, wherein X₄ is G or Q, wherein X₅ is S, D or H, wherein X₆ is G, S L, or A, wherein X₇ is S, G, or R, wherein X₈ is T, S, P or E, wherein X₉ is T or I, wherein X₁₀ is A or V, wherein X₁₁ is D or E, and wherein X₁₂ is S or A; X₁X₂NPX₃X₄GX₅X₆GX₇X₈ X₉X₁₀FX₁₁X₁₂ (SEQ ID NO: 564), wherein X₁ is W or R, wherein X₂ is M or L, wherein X₃ is N, Q, or K, wherein X₄ is S, A, or R, wherein X₅ is N, or Q, wherein X₆ is S, A, or T, wherein X₇ is S, Q, or Y, wherein X₈ is V or T, wherein X₉ is Q or K, wherein X₁₀ is K or N, wherein X₁₁ is R or Q, and wherein X₁₂ is G or D; TSGSGSTTYYADSVKG (SEQ ID NO: 584); WMNPNSGNSSVQKFRG (SEQ ID NO: 585); NIKQDGSEEYYVDSVKG (SEQ ID NO: 586); and TSGSGTYYADSVKG (SEQ ID NO: 841).

In various embodiments, the TREM-1 antibody comprises an antigen binding domain comprising a sequence having a heavy chain variable region comprising a HCDR3 amino acid sequence selected from the group consisting of X₁X₂X₃X₄X₅X₆ X₇ F X₈YYX₉ (SEQ ID NO: 557), wherein X₁ is V, E, A or G, wherein X₂ is A, F, Y or G, wherein X₃ is G, S, Y or W, wherein X₄ is S or R, wherein X₅ is absent or is N, wherein X₆ is F, S, Y, or absent, wherein X₇ is L or F or absent, wherein Xₐ is D or E, and wherein X₉ is Y, H or S; X₁X₂X₃X₄ X₅X₆ X₇X₈ X₉X₁₀X₁₁X₁₂FX₁₃X₁₄ (SEQ ID NO: 565); wherein X₁ is G, L or R, wherein X₂ is G, I, or R, wherein X₃ is Y, R, I, G, or A, wherein X₄ is T, S, Y, or V, wherein X₅ is S or Y, wherein X₆ is S, A, I, or R, wherein X₇ is W, A, or S, wherein X₈ is absent or is S, wherein X₉ is absent or is F, W, or Y, wherein X₁₀ is R, S, H, K, or E, wherein X₁₁ is W, H, Y, or F, wherein X₁₂ is Y, V, A, or S, wherein X₁₃ is D or Q, and wherein X₁₄ is L, Y, I, or H; VAGSNFLFDY (SEQ ID NO: 842); GGYTSSWRWYFDL (SEQ ID NO: 843); GGYTSSWSRWYFDL (SEQ ID NO: 844); and DYGDSFDY (SEQ ID NO: 845).

In a related embodiment, the residues of the framework are altered. The heavy chain framework regions which can be altered lie within regions designated H-FR1, H-FR2, H-FR3 and H-FR4, which surround the heavy chain CDR residues, and the residues of the light chain framework regions which can be altered lie within the regions designated L-FR1, L-FR2, L-FR3 and L-FR4, which surround the light chain CDR residues. An amino acid within the framework region may be replaced, for example, with any suitable amino acid identified in a human framework or human consensus framework. It is further contemplated that the framework regions may be altered, but the antigen binding protein or antibodies described herein retain the CDRs, LCDR1-3 and/or HCDR-3, of the parent antibodies.

Antibody fragments comprise a portion of an intact full length antibody, preferably an antigen binding or variable region of the intact antibody. Examples of antibody fragments include Fab, Fab′, F(ab′)2, Fcab, and Fv fragments; diabodies; linear antibodies; single-chain antibody molecules (e.g., scFv); multispecific antibody fragments such as bispecfic, trispecific, etc. antibodies (e.g., diabodies, triabodies, tetrabodies); minibody; chelating recombinant antibody; tribodies or bibodies; intrabodies; nanobodies; binding-domain immunoglobulin fusion proteins; camelized antibodies; VHH containing antibodies; and other polypeptides formed from antibody fragments. See for example Holliger & Hudson, 2005 Nat. Biotech. 23:1126-36; Eyer & Hruska, Veterinarni Medicina 57:439-513, 2012.

The antigen binding compounds of the present disclosure preferably retain binding affinity of 10⁻⁸, 10⁻¹, 10⁻¹⁰, 10⁻¹¹, 10⁻¹², 10⁻¹³, 10⁻¹⁴, 10⁻¹⁵ M or less for TREM-1, as measured by surface plasmon resonance or KinexA. The antigen binding compounds of the present disclosure have binding affinity from TREM-1 from 10⁻⁹ to 10⁻¹², or 10⁻¹⁰ to 10⁻¹³, or 10⁻¹⁰ to 10⁻¹⁵ M. The SPR assay is carried out using standard methods, for example, at 25° C. (room temperature).

In various embodiments, the antibody is an IgG1 antibody. In certain embodiments, the antibody is an IgG1z antibody.

Examples of full length antibodies that bind TREM-1 are provided herein having the heavy chain amino acid sequences set out in SEQ ID NOS: 6, 26, 46, 66, 86, 106, 126, 146, 166, 186, 206, 226, 246, 266, and 540; and the light chain amino acid sequences set out in SEQ ID NOS: 5, 25, 45, 65, 85, 105, 125, 145, 165, 185, 205, 225, 245, 265, and 539.

In various embodiments, the TREM-1 antigen-binding protein is selected from the group consisting of a human antibody, a humanized antibody, a chimeric antibody, a monoclonal antibody, a recombinant antibody, a Fab, a F(ab′)2, a Fab2, a monovalent IgG, an scFv, an scFv-Fc, an IgG1 antibody, an IgG2 antibody, an IgG3 antibody, and an IgG4 antibody.

In various embodiments, the antigen-binding moiety is an IgG. In various embodiments, the antigen-binding moiety is an IgG2 antibody. In various embodiments, the antigen-binding moiety is an IgG1 antibody. In various embodiments, the antibody is an IgG1z antibody. In various embodiments, the antigen-binding moiety is a monovalent IgG. In various embodiments, the heavy chain constant region of the antigen-binding moiety is selected from heavy chain constant regions of an IgG, IgM, IgA, IgD, IgE, fragments thereof, combinations thereof, and modifications thereof in which one to ten heavy chain framework amino acids are replaced with corresponding amino acid(s) from another human antibody constant region.

In various embodiments, the antigen binding protein binds its antigen with a binding affinity of 10⁻⁸ M to 10⁻¹⁵ M or 10⁻⁸ M to 10⁻¹² M, or 10⁻⁸ M, 10⁻⁹ M, 10⁻¹⁰ M, 10⁻¹ M, 10⁻¹² M, 10⁻¹³ M, 10⁻¹⁴ M, or 10⁻¹⁵ M.

In various embodiments, the anti-TREM-1 antigen binding protein comprises:
a. a light chain variable domain comprising:
   i. a light chain CDR1 comprising an amino acid sequence selected from SEQ ID NOS: 10, 30, 50, 70, 90, 110, 130, 150, 170, 190, 210, 230, 250, 270, and 544;
   ii. a light chain CDR2 comprising an amino acid sequence selected from SEQ ID NOS: 11, 31, 51, 71, 91, 111, 131, 151, 171, 191, 211, 231, 251, 271, and 545;
   iii. a light chain CDR3 comprising an amino acid sequence selected from SEQ ID NOS: 12, 32, 52, 72, 92, 112, 132, 152, 172, 192, 212, 232, 252, 272, and 546; and
b. comprises a heavy chain variable domain comprising:
   i. a heavy chain CDR1 comprising an amino acid sequence selected from SEQ ID NOS: 16, 36, 56, 76, 96, 116, 136, 156, 176, 196, 216, 236, 256, 276, and 550;
   ii. a heavy chain CDR2 comprising an amino acid sequence selected from SEQ ID NOS: 17, 37, 57, 77, 97, 117, 137, 157, 177, 197, 217, 237, 257, 277, and 551; and
   iii. a heavy chain CDR3 comprising an amino acid sequence selected from SEQ ID NOS: 18, 38, 58, 78, 98, 118, 138, 158, 178, 198, 218, 238, 258, 278, and 552.

In various embodiments of the antigen binding protein,
a. the light chain CDR1 sequence is set out in SEQ ID NO: 10, 30, 50, 90, 130, 150, or 270;
b. the light chain CDR2 sequence is set out in SEQ ID NO: 11, 31, 51, 91, 131, 151, or 271;
c. the light chain CDR3 sequence is set out in SEQ ID NO: 12, 32, 52, 92, 132, 152, or 272
d. the heavy chain CDR1 sequence is set out in SEQ ID NO: 16, 36, 56, 96, 136, 156, or 276;
e. the heavy chain CDR2 sequence is set out in SEQ ID NO: 17, 37, 57, 97, 137, 157, or 277; and
f. the heavy chain CDR3 sequence is set out in SEQ ID NO: 18, 38, 58, 98, 138, 158, or 278.

In various embodiments of the antigen binding protein,
a. the light chain CDR1 sequence is set out in SEQ ID NO: 30 or 90;
b. the light chain CDR2 sequence is set out in SEQ ID NO: 31 or 91;
c. the light chain CDR3 sequence is set out in SEQ ID NO: 32 or 92;
d. the heavy chain CDR1 sequence is set out in SEQ ID NO: 36 or 096;
e. the heavy chain CDR2 sequence is set out in SEQ ID NO: 37 or 97; and
f. the heavy chain CDR3 sequence is set out in SEQ ID NOS: 38 or 98.

In various embodiments, the antigen binding protein comprises the heavy chain (HC) CDR-H1, CDR-H2, and CDR-H3 of SEQ ID NO:22, and the light chain (LC) CDR-L1, CDR-L2, and CDR-L3 of SEQ ID NO:21. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:42, and the light chain (LC) CDR-L1, CDR-L2, and CDR-L3 of SEQ ID NO:41. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:62, and the light chain (LC) CDR-L1, CDR-L2, and CDR-L3 of SEQ ID NO:61. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:82, and the light chain (LC) CDR-L1, CDR-L2, and CDR-L3 of SEQ ID NO:81. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:102, and the light chain (LC) CDR-L1, CDR-L2, and CDR-L3 of SEQ ID NO:101. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:122, and the light chain (LC) CDR-L1, CDR-L2, and CDR-L3 of SEQ ID NO:121. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:142, and the light chain (LC) CDR-L1, CDR-L2, and CDR-L3 of SEQ ID NO:141. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:162, and the light chain (LC) CDR-L1, CDR-L2, and CDR-L3 of SEQ ID NO:161. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:182, and the light chain (LC) CDR-L1, CDR-L2, and CDR-L3 of SEQ ID NO:181. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:202, and the light chain (LC) CDR-L1, CDR-L2, and CDR-L3 of SEQ ID NO:201. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:222, and the light chain (LC) CDR-L1, CDR-L2, and CDR-L3 of SEQ ID NO:221. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:242, and the light chain (LC) CDR-L1, CDR-L2, and CDR-L3 of SEQ ID NO:241. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:262, and the light chain (LC) CDR-L1, CDR-L2, and CDR-L3 of SEQ ID NO:261. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:282, and the light chain (LC) CDR-L1, CDR-L2, and CDR-L3 of SEQ ID NO:281. In exemplary embodiments, the CDRs are defined according to Kabat, Chothia, AbM, contact, or IMGT.

In various embodiments, the antigen binding protein comprises the heavy chain (HC) CDR-H1, CDR-H2, and CDR-H3 of SEQ ID NO:540, and the light chain (LC) CDR-L1, CDR-L2, and CDR-L3 of SEQ ID NO:539. In exemplary embodiments, the CDRs are defined according to Kabat, Chothia, AbM, contact, or IMGT.

In various embodiments, the anti-TREM-1 antigen-binding protein comprises:
a. a light chain variable domain comprising an amino acid sequence selected from the group consisting of:
   i. a sequence at least 80% identical to an amino acid sequence selected from SEQ ID NOS: 21, 41, 61, 81, 101, 121, 141, 161, 181, 201, 221, 241, 261, 281, and 539;
   ii. a sequence encoded by a polynucleotide sequence that is at least 80% identical to a nucleic acid sequence selected from SEQ ID NOS: 19, 39, 59, 79, 99, 119, 139, 159, 179, 199, 219, 239, 259, 279, and 537;
   iii. a sequence encoded by a polynucleotide that hybridizes under moderately stringent conditions to the complement of a polynucleotide consisting of a nucleic acid sequence selected from SEQ ID NOS: 19, 39, 59, 79, 99, 119, 139, 159, 179, 199, 219, 239, 259, 279, and 537; and
b. a heavy chain variable domain comprising an amino acid sequence selected from the group consisting of:
   i. a sequence that is at least 80% identical to an amino acid sequence selected from SEQ ID NOS: 22, 42, 62, 82, 102. 122, 142, 162, 182, 202, 222, 242, 262, 282, and 540;
   ii. a sequence encoded by a polynucleotide sequence that is at least 80% identical to an amino acid sequence selected from SEQ ID NOS: 20, 40, 60, 80, 100, 120, 140,160, 180, 200, 220, 240, 260, 280, and 538; and
   iii. a sequence encoded by a polynucleotide that hybridizes under moderately stringent conditions to the complement of a polynucleotide consisting of a nucleic sequence selected from SEQ ID NOS: 20, 40, 60, 80, 100, 120, 140, 160, 180, 200, 220, 240, 260, 280, and 538.

In various embodiments, the anti-TREM-1 antigen-binding protein comprises:
a. a light chain variable domain comprising an amino acid sequence selected from the group consisting of:
   i. a sequence at least 80% identical to an amino acid sequence selected from SEQ ID NOS: 21, 41, 61, 101, 141, 161, and 281;
   ii. a sequence encoded by a polynucleotide sequence that is at least 80% identical to a nucleic acid sequence selected from SEQ ID NOS: 19, 39, 59, 99, 139, 159, and 279;
   iii. a sequence encoded by a polynucleotide that hybridizes under moderately stringent conditions to the complement of a polynucleotide consisting of a nucleic acid sequence selected from SEQ ID NOS: 19, 39, 59, 99, 139, 159, and 279; and
b. a heavy chain variable domain comprising an amino acid sequence selected from the group consisting of:
   i. a sequence that is at least 80% identical to an amino acid sequence selected from SEQ ID NOS: 22, 42, 62, 102, 142, 162, and 282;
   ii. a sequence encoded by a polynucleotide sequence that is at least 80% identical to an amino acid sequence selected from SEQ ID NOS: 20, 40, 60, 100, 140, 160, and 280;
   iii. a sequence encoded by a polynucleotide that hybridizes under moderately stringent conditions to the complement of a polynucleotide consisting of a nucleic sequence selected from SEQ ID NOS: 20, 40, 60, 100, 140, 160, and 280.

In various embodiments, the anti-TREM-1 antigen-binding protein comprises:
a. a light chain variable domain comprising an amino acid sequence selected from the group consisting of:
   i. a sequence at least 80% identical to an amino acid sequence selected from SEQ ID NOS: 41 and 101;
   ii. a sequence encoded by a polynucleotide sequence that is at least 80% identical to a nucleic acid sequence selected from SEQ ID NOS: 39 and 99;
   iii. a sequence encoded by a polynucleotide that hybridizes under moderately stringent conditions to the complement of a polynucleotide consisting of a nucleic acid sequence selected from SEQ ID NOS: 39 and 99; and
b. a heavy chain variable domain comprising an amino acid sequence selected from the group consisting of:
   i. a sequence that is at least 80% identical to an amino acid sequence selected from SEQ ID NOS: 42 and 102;
   ii. a sequence encoded by a polynucleotide sequence that is at least 80% identical to an amino acid sequence selected from SEQ ID NOS: 40 and 100;
   iii. a sequence encoded by a polynucleotide that hybridizes under moderately stringent conditions to the complement of a polynucleotide consisting of a nucleic sequence selected from SEQ ID NOS: 40 and 100.

In various embodiments, the amino acid sequences can be 80%, 85%, 90%, 91%, 92%, 93%, 94%, 95%, 96%, 97%, 98% or 99% identical to SEQ ID NOs: 21, 41, 61, 81, 101, 121, 141, 161, 181, 201, 221, 241, 261, 281, and 539 and SEQ ID NO: 22, 42, 62, 82, 102. 122, 142, 162, 182, 202, 222, 242, 262, 282, and 540.

In various embodiments, the antigen binding protein comprises an amino acid sequence at least 90% identical to a heavy chain variable region amino acid sequence selected from SEQ ID NOS: 22, 42, 62, 82, 102. 122, 142, 162, 182, 202, 222, 242, 262, 282, and 540.

In various embodiments, the antigen-binding protein comprises an amino acid sequence at least 90% identical to a light chain variable region amino acid sequence selected from SEQ ID NOs: 21, 41, 61, 81, 101, 121, 141, 161, 181, 201, 221, 241, 261, 281, and 539.

In various embodiments, the antigen binding protein comprises a heavy chain amino acid sequence at least 90% identical to a sequence selected from SEQ ID NOS: 22, 42, 62, 102, 142, 162, and 282 and a light chain amino acid sequence at least 90% identical to a sequence selected from SEQ ID NOS: 21, 41, 61, 101, 141, 161, and 281.

In various embodiments, the antigen binding protein comprises a heavy chain amino acid sequence at least 90% identical to a sequence selected from SEQ ID NOS:4 and 102, and a light chain amino acid sequence at least 90% identical to a sequence selected from SEQ ID NOS: 41 and 101.

In various embodiments, the antigen binding protein comprises a heavy chain variable domain (VH) comprising three CDRs that in combination are at least 85% identical to the three heavy chain CDRs in SEQ ID NO:22; and a light chain variable domain (VL) comprising three CDRs that in combination are at least 85% identical to the three light chain CDRs in SEQ ID NO:21. In various embodiments, the antigen binding protein comprises a VH comprising three CDRs that in combination are at least 90% identical to the three heavy chain CDRs in SEQ ID NO:22; and a VL comprising three CDRs that in combination are at least 90% identical to the three light chain CDRs in SEQ ID NO:21. In various embodiments, the antigen binding protein comprises a VH comprising three CDRs that in combination are at least 95% identical to the three heavy chain CDRs in SEQ ID NO:22; and a VL comprising three CDRs that in combination are at least 95% identical to the three light chain CDRs in SEQ ID NO:21. The percent identity is determined by overall percent identity when combining three CDR sequences together and align them against the sequences disclosed herein. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:42; and a light chain variable domain (VL) comprising three CDRs that in combination are at least 85% identical to the three light chain CDRs in SEQ ID NO:41. In various embodiments, the antigen binding protein comprises a VH comprising three CDRs that in combination are at least 90% identical to the three heavy chain CDRs in SEQ ID NO:42; and a VL comprising three CDRs that in combination are at least 90% identical to the three light chain CDRs in SEQ ID NO:41. In various embodiments, the antigen binding protein comprises a VH comprising three CDRs that in combination are at least 95% identical to the three heavy chain CDRs in SEQ ID NO:42; and a VL comprising three CDRs that in combination are at least 95% identical to the three light chain CDRs in SEQ ID NO:41. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:62; and a light chain variable domain (VL) comprising three CDRs that in combination are at least 85% identical to the three light chain CDRs in SEQ ID NO:61. In various embodiments, the antigen binding protein comprises a VH comprising three CDRs that in combination are at least 90% identical to the three heavy chain CDRs in SEQ ID NO:62; and a VL comprising three CDRs that in combination are at least 90% identical to the three light chain CDRs in SEQ ID NO:61. In various embodiments, the antigen binding protein comprises a VH comprising three CDRs that in combination are at least 95% identical to the three heavy chain CDRs in SEQ ID NO:62; and a VL comprising three CDRs that in combination are at least 95% identical to the three light chain CDRs in SEQ ID NO:61. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:82; and a light chain variable domain (VL) comprising three CDRs that in combination are at least 85% identical to the three light chain CDRs in SEQ ID NO:81. In various embodiments, the antigen binding protein comprises a VH comprising three CDRs that in combination are at least 90% identical to the three heavy chain CDRs in SEQ ID NO:82; and a VL comprising three CDRs that in combination are at least 90% identical to the three light chain CDRs in SEQ ID NO:81. In various embodiments, the antigen binding protein comprises a VH comprising three CDRs that in combination are at least 95% identical to the three heavy chain CDRs in SEQ ID NO:82; and a VL comprising three CDRs that in combination are at least 95% identical to the three light chain CDRs in SEQ ID NO:81. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:102; and a light chain variable domain (VL) comprising three CDRs that in combination are at least 85% identical to the three light chain CDRs in SEQ ID NO:101. In various embodiments, the antigen binding protein comprises a VH comprising three CDRs that in combination are at least 90% identical to the three heavy chain CDRs in SEQ ID NO:102; and a VL comprising three CDRs that in combination are at least 90% identical to the three light chain CDRs in SEQ ID NO:101. In various embodiments, the antigen binding protein comprises a VH comprising three CDRs that in combination are at least 95% identical to the three heavy chain CDRs in SEQ ID NO:102; and a VL comprising three CDRs that in combination are at least 95% identical to the three light chain CDRs in SEQ ID NO:101. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:122; and a light chain variable domain (VL) comprising three CDRs that in combination are at least 85% identical to the three light chain CDRs in SEQ ID NO:121. In various embodiments, the antigen binding protein comprises a VH comprising three CDRs that in combination are at least 90% identical to the three heavy chain CDRs in SEQ ID NO:122; and a VL comprising three CDRs that in combination are at least 90% identical to the three light chain CDRs in SEQ ID NO:121. In various embodiments, the antigen binding protein comprises a VH comprising three CDRs that in combination are at least 95% identical to the three heavy chain CDRs in SEQ ID NO:122; and a VL comprising three CDRs that in combination are at least 95% identical to the three light chain CDRs in SEQ ID NO:121. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:122; and a light chain variable domain (VL) comprising three CDRs that in combination are at least 85% identical to the three light chain CDRs in SEQ ID NO:121. In various embodiments, the antigen binding protein comprises a VH comprising three CDRs that in combination are at least 90% identical to the three heavy chain CDRs in SEQ ID NO:122; and a VL comprising three CDRs that in combination are at least 90% identical to the three light chain CDRs in SEQ ID NO:121. In various embodiments, the antigen binding protein comprises a VH comprising three CDRs that in combination are at least 95% identical to the three heavy chain CDRs in SEQ ID NO:122; and a VL comprising three CDRs that in combination are at least 95% identical to the three light chain CDRs in SEQ ID NO:121. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:142; and a light chain variable domain (VL) comprising three CDRs that in combination are at least 85% identical to the three light chain CDRs in SEQ ID NO:141. In various embodiments, the antigen binding protein comprises a VH comprising three CDRs that in combination are at least 90% identical to the three heavy chain CDRs in SEQ ID NO:142; and a VL comprising three CDRs that in combination are at least 90% identical to the three light chain CDRs in SEQ ID NO:141. In various embodiments, the antigen binding protein comprises a VH comprising three CDRs that in combination are at least 95% identical to the three heavy chain CDRs in SEQ ID NO:142; and a VL comprising three CDRs that in combination are at least 95% identical to the three light chain CDRs in SEQ ID NO:141. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:162; and a light chain variable domain (VL) comprising three CDRs that in combination are at least 85% identical to the three light chain CDRs in SEQ ID NO:161. In various embodiments, the antigen binding protein comprises a VH comprising three CDRs that in combination are at least 90% identical to the three heavy chain CDRs in SEQ ID NO:162; and a VL comprising three CDRs that in combination are at least 90% identical to the three light chain CDRs in SEQ ID NO:161. In various embodiments, the antigen binding protein comprises a VH comprising three CDRs that in combination are at least 95% identical to the three heavy chain CDRs in SEQ ID NO:162; and a VL comprising three CDRs that in combination are at least 95% identical to the three light chain CDRs in SEQ ID NO:161. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:182; and a light chain variable domain (VL) comprising three CDRs that in combination are at least 85% identical to the three light chain CDRs in SEQ ID NO:181. In various embodiments, the antigen binding protein comprises a VH comprising three CDRs that in combination are at least 90% identical to the three heavy chain CDRs in SEQ ID NO:182; and a VL comprising three CDRs that in combination are at least 90% identical to the three light chain CDRs in SEQ ID NO:181. In various embodiments, the antigen binding protein comprises a VH comprising three CDRs that in combination are at least 95% identical to the three heavy chain CDRs in SEQ ID NO:182; and a VL comprising three CDRs that in combination are at least 95% identical to the three light chain CDRs in SEQ ID NO:181. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:202; and a light chain variable domain (VL) comprising three CDRs that in combination are at least 85% identical to the three light chain CDRs in SEQ ID NO:201. In various embodiments, the antigen binding protein comprises a VH comprising three CDRs that in combination are at least 90% identical to the three heavy chain CDRs in SEQ ID NO:202; and a VL comprising three CDRs that in combination are at least 90% identical to the three light chain CDRs in SEQ ID NO:201. In various embodiments, the antigen binding protein comprises a VH comprising three CDRs that in combination are at least 95% identical to the three heavy chain CDRs in SEQ ID NO:202; and a VL comprising three CDRs that in combination are at least 95% identical to the three light chain CDRs in SEQ ID NO:201. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:222; and a light chain variable domain (VL) comprising three CDRs that in combination are at least 85% identical to the three light chain CDRs in SEQ ID NO:221. In various embodiments, the antigen binding protein comprises a VH comprising three CDRs that in combination are at least 90% identical to the three heavy chain CDRs in SEQ ID NO:222; and a VL comprising three CDRs that in combination are at least 90% identical to the three light chain CDRs in SEQ ID NO:221. In various embodiments, the antigen binding protein comprises a VH comprising three CDRs that in combination are at least 95% identical to the three heavy chain CDRs in SEQ ID NO:222; and a VL comprising three CDRs that in combination are at least 95% identical to the three light chain CDRs in SEQ ID NO:221. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:242; and a light chain variable domain (VL) comprising three CDRs that in combination are at least 85% identical to the three light chain CDRs in SEQ ID NO:241. In various embodiments, the antigen binding protein comprises a VH comprising three CDRs that in combination are at least 90% identical to the three heavy chain CDRs in SEQ ID NO:242; and a VL comprising three CDRs that in combination are at least 90% identical to the three light chain CDRs in SEQ ID NO:241. In various embodiments, the antigen binding protein comprises a VH comprising three CDRs that in combination are at least 95% identical to the three heavy chain CDRs in SEQ ID NO:242; and a VL comprising three CDRs that in combination are at least 95% identical to the three light chain CDRs in SEQ ID NO:241. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:262; and a light chain variable domain (VL) comprising three CDRs that in combination are at least 85% identical to the three light chain CDRs in SEQ ID NO:261. In various embodiments, the antigen binding protein comprises a VH comprising three CDRs that in combination are at least 90% identical to the three heavy chain CDRs in SEQ ID NO:262; and a VL comprising three CDRs that in combination are at least 90% identical to the three light chain CDRs in SEQ ID NO:261. In various embodiments, the antigen binding protein comprises a VH comprising three CDRs that in combination are at least 95% identical to the three heavy chain CDRs in SEQ ID NO:262; and a VL comprising three CDRs that in combination are at least 95% identical to the three light chain CDRs in SEQ ID NO:261. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:282; and a light chain variable domain (VL) comprising three CDRs that in combination are at least 85% identical to the three light chain CDRs in SEQ ID NO:281. In various embodiments, the antigen binding protein comprises a VH comprising three CDRs that in combination are at least 90% identical to the three heavy chain CDRs in SEQ ID NO:282; and a VL comprising three CDRs that in combination are at least 90% identical to the three light chain CDRs in SEQ ID NO:281. In various embodiments, the antigen binding protein comprises a VH comprising three CDRs that in combination are at least 95% identical to the three heavy chain CDRs in SEQ ID NO:282; and a VL comprising three CDRs that in combination are at least 95% identical to the three light chain CDRs in SEQ ID NO:281. The antigen binding protein may further bind to its target (TREM-1) with a KD value of 100 nM or less, or a KD value of 50 nM or less.

In various embodiments, the antigen binding protein comprises a heavy chain variable domain (VH) comprising three CDRs that in combination are at least 85% identical to the three heavy chain CDRs in SEQ ID NO:540; and a light chain variable domain (VL) comprising three CDRs that in combination are at least 85% identical to the three light chain CDRs in SEQ ID NO:539. In various embodiments, the antigen binding protein comprises a VH comprising three CDRs that in combination are at least 90% identical to the three heavy chain CDRs in SEQ ID NO:540; and a VL comprising three CDRs that in combination are at least 90% identical to the three light chain CDRs in SEQ ID NO:539. In various embodiments, the antigen binding protein comprises a VH comprising three CDRs that in combination are at least 95% identical to the three heavy chain CDRs in SEQ ID NO:540; and a VL comprising three CDRs that in combination are at least 95% identical to the three light chain CDRs in SEQ ID NO:539. The antigen binding protein may further bind to its target (TREM-1) with a KD value of 100 nM or less, or a KD value of 50 nM or less.

In various embodiments, the disclosure provides a method for treating cardiovascular disease (e.g., atherosclerosis or myocardial infarction) comprising administering an antigen binding protein, wherein the antigen binding protein: binds to human TrigGering Receptor Expressed on Myeloid cells 1 (TREM-1) and comprises a set of CDR sequences selected from:
i) SEQ ID NO: 10 (LCDR1), SEQ ID NO: 11 (LCDR2), SEQ ID NO: 12 (LCDR3), SEQ ID NO: 16 (HCDR1), SEQ ID NO: 17 (HCDR2) and SEQ ID NO: 18 (HCDR3);
ii) SEQ ID NO: 30 (LCDR1), SEQ ID NO: 31 (LCDR2), SEQ ID NO: 32 (LCDR3), SEQ ID NO: 36 (HCDR1), SEQ ID NO: 37 (HCDR2) and SEQ ID NO: 38 (HCDR3);
iii) SEQ ID NO: 50 (LCDR1), SEQ ID NO: 51 (LCDR2), SEQ ID NO: 52 (LCDR3), SEQ ID NO: 56 (HCDR1), SEQ ID NO: 57 (HCDR2) and SEQ ID NO: 58 (HCDR3);
iv) SEQ ID NO: 70 (LCDR1), SEQ ID NO: 71 (LCDR2), SEQ ID NO: 72 (LCDR3), SEQ ID NO: 76 (HCDR1), SEQ ID NO: 77 (HCDR2) and SEQ ID NO: 78 (HCDR3);
v) SEQ ID NO: 90 (LCDR1), SEQ ID NO: 91 (LCDR2), SEQ ID NO: 92 (LCDR3), SEQ ID NO: 96 (HCDR1), SEQ ID NO: 97 (HCDR2) and SEQ ID NO: 98 (HCDR3);
vi) SEQ ID NO: 110 (LCDR1), SEQ ID NO: 111 (LCDR2), SEQ ID NO: 112 (LCDR3), SEQ ID NO: 116 (HCDR1), SEQ ID NO: 117 (HCDR2) and SEQ ID NO: 118 (HCDR3);
vii) SEQ ID NO: 130 (LCDR1), SEQ ID NO: 131 (LCDR2), SEQ ID NO: 132 (LCDR3), SEQ ID NO: 136 (HCDR1), SEQ ID NO: 137 (HCDR2) and SEQ ID NO: 138 (HCDR3);
viii) SEQ ID NO: 150 (LCDR1), SEQ ID NO: 151 (LCDR2), SEQ ID NO: 152 (LCDR3), SEQ ID NO: 156 (HCDR1), SEQ ID NO: 157 (HCDR2) and SEQ ID NO: 158 (HCDR3);
ix) SEQ ID NO: 170 (LCDR1), SEQ ID NO: 171 (LCDR2), SEQ ID NO: 172 (LCDR3), SEQ ID NO: 176 (HCDR1), SEQ ID NO: 177 (HCDR2) and SEQ ID NO: 178 (HCDR3);
x) SEQ ID NO: 190 (LCDR1), SEQ ID NO: 191 (LCDR2), SEQ ID NO: 192 (LCDR3), SEQ ID NO: 196 (HCDR1), SEQ ID NO: 197 (HCDR2) and SEQ ID NO: 198 (HCDR3);
xi) SEQ ID NO: 210 (LCDR1), SEQ ID NO: 211 (LCDR2), SEQ ID NO: 212 (LCDR3), SEQ ID NO: 216 (HCDR1), SEQ ID NO: 217 (HCDR2) and SEQ ID NO: 218 (HCDR3);
xii) SEQ ID NO: 230 (LCDR1), SEQ ID NO: 231 (LCDR2), SEQ ID NO: 232 (LCDR3), SEQ ID NO: 236 (HCDR1), SEQ ID NO: 237 (HCDR2) and SEQ ID NO: 238 (HCDR3);
xiii) SEQ ID NO: 250 (LCDR1), SEQ ID NO: 251 (LCDR2), SEQ ID NO: 252 (LCDR3), SEQ ID NO: 256 (HCDR1), SEQ ID NO: 257 (HCDR2) and SEQ ID NO: 258 (HCDR3);
ix) SEQ ID NO: 270 (LCDR1), SEQ ID NO: 271 (LCDR2), SEQ ID NO: 272 (LCDR3), SEQ ID NO: 276 (HCDR1), SEQ ID NO: 277 (HCDR2) and SEQ ID NO: 278 (HCDR3); and
xv) SEQ ID NO: 544 (LCDR1), SEQ ID NO: 545 (LCDR2), SEQ ID NO: 546 (LCDR3), SEQ ID NO: 547 (HCDR1), SEQ ID NO: 548 (HCDR2) and SEQ ID NO: 549 (HCDR3).

In various embodiments, the anti-TREM-1 antigen-binding protein comprises a set of CDR sequences selected from:
i) SEQ ID NO: 10 (LCDR1), SEQ ID NO: 11 (LCDR2), SEQ ID NO: 12 (LCDR3), SEQ ID NO: 16 (HCDR1), SEQ ID NO: 17 (HCDR2) and SEQ ID NO: 18 (HCDR3);
ii) SEQ ID NO: 30 (LCDR1), SEQ ID NO: 31 (LCDR2), SEQ ID NO: 32 (LCDR3), SEQ ID NO: 36 (HCDR1), SEQ ID NO: 37 (HCDR2) and SEQ ID NO: 38 (HCDR3);
iii) SEQ ID NO: 50 (LCDR1), SEQ ID NO: 51 (LCDR2), SEQ ID NO: 52 (LCDR3), SEQ ID NO: 56 (HCDR1), SEQ ID NO: 57 (HCDR2) and SEQ ID NO: 58 (HCDR3);
iv) SEQ ID NO: 90 (LCDR1), SEQ ID NO: 91 (LCDR2), SEQ ID NO: 92 (LCDR3), SEQ ID NO: 96 (HCDR1), SEQ ID NO: 97 (HCDR2) and SEQ ID NO: 98 (HCDR3);
v) SEQ ID NO: 130 (LCDR1), SEQ ID NO: 131 (LCDR2), SEQ ID NO: 132 (LCDR3), SEQ ID NO: 136 (HCDR1), SEQ ID NO: 137 (HCDR2) and SEQ ID NO: 138 (HCDR3);
vi) SEQ ID NO: 150 (LCDR1), SEQ ID NO: 151 (LCDR2), SEQ ID NO: 152 (LCDR3), SEQ ID NO: 156 (HCDR1), SEQ ID NO: 157 (HCDR2) and SEQ ID NO: 158 (HCDR3); or
vii) SEQ ID NO: 270 (LCDR1), SEQ ID NO: 271 (LCDR2), SEQ ID NO: 272 (LCDR3), SEQ ID NO: 276 (HCDR1), SEQ ID NO: 277 (HCDR2) and SEQ ID NO: 278 (HCDR3).

In various embodiments, the anti-TREM-1 antigen binding protein comprises a set of CDR sequences selected from SEQ ID NO: 30 (LCDR1), SEQ ID NO: 31 (LCDR2), SEQ ID NO: 32 (LCDR3), SEQ ID NO: 36 (HCDR1), SEQ ID NO: 37 (HCDR2) and SEQ ID NO: 38 (HCDR3); or SEQ ID NO: 90 (LCDR1), SEQ ID NO: 91 (LCDR2), SEQ ID NO: 92 (LCDR3), SEQ ID NO: 96 (HCDR1), SEQ ID NO: 97 (HCDR2) and SEQ ID NO: 98 (HCDR3).

Binding affinities of the TREM-1 antigen binding proteins can be assessed by KD. KD is the equilibrium dissociation constant, a ratio of k_{off}/kₒₙ, between the antigen binding protein and its target or antigen. KD and KA are inversely related. The KD value relates to the concentration of the antibody (the amount of antibody needed for a particular experiment) and so the lower the KD value (lower concentration needed) the higher the affinity of the antibody. In exemplary aspects, the KD value of the TREM-1 antigen binding protein to its target is about 10⁻¹ M or less, about 10⁻² M or less, about 10⁻³ M or less, about 10⁻⁴ M or less, about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, or from about 10⁻¹⁰ M to about 10⁻¹³ M.

In exemplary aspects, the KD value of the TREM-1 antigen binding protein to its target is micromolar, nanomolar, picomolar, or femtomolar. In exemplary aspects, the KD is within a range of about 10⁻⁴ to 10⁻⁶ M, or 10⁻⁷ to 10⁻⁹ M, or 10⁻¹⁰ to 10⁻¹² M, or 10⁻¹³ to 10⁻¹⁵ M. In exemplary aspects, the TREM-1 antigen binding protein binds to its target with a KD value of: about 1 uM or less, about 900 nM or less, about 800 nM or less, about 700 nM or less, about 600 nM or less, about 500 nM or less, about 400 nM or less, about 300 nM or less, about 200 nM or less, about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 5 nM or less, about 2 nM or less, about 1 nM or less, about 900 pM or less, about 800 pM or less, about 700 pM or less, about 600 pM or less, about 500 pM or less, about 400 pM or less, about 300 pM or less, about 250 pM or less, about 200 pM or less, about 150 pM or less, about 100 pM or less, about 50 pM or less, about 40 pM or less, about 30 pM or less, about 25 pM or less, about 20 pM or less, about 15 pM or less, about 10 pM or less, about 5 pM or less, or about 1 pM or less.

KD values can be determined using methods well established in the art. One exemplary method for measuring KD is surface plasmon resonance (SPR), a method well-known in the art (e.g., Nguyen et al. Sensors (Basel). 2015 May 5; 15(5):10481-510). KD value may be measured by SPR using a biosensor system such as a BIACORE® system. BIAcore kinetic analysis comprises analyzing the binding and dissociation of an antigen from chips with immobilized molecules (e.g. molecules comprising epitope binding domains), on their surface. Another well-known method in the art for determining the KD of a protein is by using Bio-Layer Interferometry (e.g., Shah et al. J Vis Exp. 2014; (84): 51383). KD value may be measured by Bio-Layer Interferometry using OCTET® technology (Octet QKe system, ForteBio). Alternatively or in addition, a KinExA® (Kinetic Exclusion Assay) assay, available from Sapidyne Instruments (Boise, Id.) can also be used. Any method known in the art for assessing the binding affinity between two binding partners is encompassed herein.

In some aspects, the KD value is measured by surface plasmon resonance (SPR). Antigen (TREM-1) may be immobilized, e.g., on a solid surface. The antigen may be immobilized to a chip, for example by covalent coupling (such as amine coupling). The chip may be a CM5 sensor chip. As the analyte binds to the ligand the accumulation of protein on the sensor surface causes an increase in refractive index. This refractive index change is measured in real time (sampling in a kinetic analysis experiment is taken every 0.1 s), and the result plotted as response units (RU) versus time (termed a sensorgram). A response (background response) will also be generated if there is a difference in the refractive indices of the running and sample buffers. This background response must be subtracted from the sensorgram to obtain the actual binding response. The background response is recorded by injecting the analyte through a control or reference flow cell, which has no ligand or an irrelevant ligand immobilized to the sensor surface. The real-time measurement of association and dissociation of a binding interaction allows for the calculation of association and dissociation rate constants and the corresponding affinity constants. One RU represents the binding of 1 pg of protein per square mm. More than 50 pg per square mm of analyte binding is generally needed in practice to generate good reproducible responses.

Dissociation of the antigen binding protein from the antigen may be monitored for about 3600 seconds. The SPR analysis may be conducted, and the data collected at between about 15° C. and about 37° C. The SPR analysis may be conducted, and the data collected at between about 25° C. and 37° C. The SPR analysis may be conducted, and the data collected at about 37° C. The SPR analysis may be conducted, and the data collected at 37° C. The KD value may be measured by SPR using a BIAcore T200 instrument. The SPR rates and affinities may be determined by fitting resulting sensorgram data to a 1:1 model in BIAcore T200 Evaluation software version 1.0. The collection rate may be about 1 Hz.

Another method for determining the KD of an antibody is by using Bio-Layer Interferometry (BLI), typically using OCTET® technology (Octet QKe system, ForteBio). In some embodiments, biosensor analysis is used. Typically, one interactant is immobilized on the surface of the biosensor (“ligand,” such as an antigen binding protein) and the other remains in solution (“analyte”, such as an antigen). The assay begins with an initial baseline or equilibration step using assay buffer. Next, a ligand (such as an antigen binding protein) is immobilized on the surface of the biosensor (loading), either by direct immobilization or capture-based method. After ligand immobilization, biosensors are dipped into buffer solution for a baseline step to assess assay drift and determine loading level of ligand. After the baseline step, biosensors are dipped into a solution containing the ligand's binding partner, the analyte (association). In this step, the binding interaction of the analyte to the immobilized ligand is measured. Following analyte association, the biosensor is dipped into buffer solution without analyte, and the bound analyte is allowed to come off the ligand (dissociation). The series of assay steps is then repeated on new or regenerated biosensors for each analyte being tested. Each binding response is measured and reported in real time on a sensorgram trace. The instrument may be Octet QKe system, Octet RED96 system, Octet QK384 system, or RED384 system.

### Nucleic Acid Molecules

The disclosure also provides isolated nucleic acids encoding the antigen binding proteins described herein, which includes, for instance, the antigen binding protein light chain, light chain variable region, light chain constant region, antigen binding protein heavy chain, heavy chain variable region, heavy chain constant region, linkers, fusion proteins, and any and all components and combinations thereof. Nucleic acids of the invention include nucleic acids having at least 80%, more preferably at least about 90%, more preferably at least about 95%, and most preferably at least about 98% homology to nucleic acids of the invention. The terms “percent similarity”, “percent identity” and “percent homology” when referring to a particular sequence are used as set forth in the University of Wisconsin GCG® software program. Nucleic acids of the disclosure also include complementary nucleic acids. In some instances, the sequences will be fully complementary (no mismatches) when aligned. In other instances, there may be up to about a 20% mismatch in the sequences. In some embodiments of the invention are provided nucleic acids encoding both a heavy chain and a light chain of an antibody of the disclosure.

Nucleic acids of the disclosure can be cloned into a vector, such as a plasmid, cosmid, bacmid, phage, artificial chromosome (BAC, YAC) or virus, into which another genetic sequence or element (either DNA or RNA) may be inserted so as to bring about the replication of the attached sequence or element. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the invention may further comprise regulatory sequences, for example, an internal ribosomal entry site. The expression vector can be introduced into a cell by transfection, for example.

Also provided is an expression vector comprising the following operably linked elements; a transcription promoter; a first nucleic acid molecule encoding an the heavy chain of an antigen binding protein, antibody or antigen-binding fragment of the disclosure; a second nucleic acid molecule encoding the light chain of an antigen binding protein, antibody or antigen-binding fragment of the disclosure; and a transcription terminator. In another embodiment, the present disclosure provides an expression vector comprising the following operably linked elements; a first transcription promoter; a first nucleic acid molecule encoding the heavy chain of an antigen binding protein, antibody or antigen-binding fragment of the disclosure; a first transcription terminator; a second transcription promoter optionally, a second nucleic acid molecule encoding the light chain of an antigen binding protein, antibody or antigen-binding fragment of the disclosure; and a second transcription terminator.

A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. For instance, in some embodiments, signal peptide sequences may be appended/fused to the amino terminus of any of the antigen binding protein, antibody or antigen binding fragment thereof polypeptide sequences described herein

Recombinant host cells comprising such vectors and expressing the antigen binding protein heavy and light chains are also provided. The recombinant host cell may be a prokaryotic cell, for example an E. coli cell, or a eukaryotic cell, for example a mammalian cell or a yeast cell. Yeast cells include Saccharomyces cerevisiae, Schizosaccharomvces pombe, and Pichia pastoris cells. Mammalian cells include VERO, HeLa, Chinese hamster Ovary (CHO), W138, baby hamster kidney (BHK), COS-7, MDCK, human embryonic kidney line 293, normal dog kidney cell lines, normal cat kidney cell lines, monkey kidney cells, African green monkey kidney cells, COS cells, and non-tumorigenic mouse myoblast G8 cells, fibroblast cell lines, myeloma cell lines, mouse NIH/3T3 cells, LMTK31 cells, mouse sertoli cells, human cervical carcinoma cells, buffalo rat liver cells, human lung cells, human liver cells, mouse mammary tumor cells, TRI cells, MRC 5 cells, and FS4 cells. Recombinant protein-producing cells of the disclosure also include any insect expression cell line known, such as for example, Spodoptera frugiperda cells. In one embodiment, the cells are mammalian cells. In a certain embodiment, the mammalian cells are CHO cells.

In a certain embodiment, the mammalian cells are HEK 293 cells.

Protein purification methods are known in the art and utilized herein for recovery of recombinant proteins from cell culture media. For example, methods of protein and antibody purification are known in the art and can be employed with production of the antibodies of the present disclosure. In some embodiments, methods for protein and antibody purification include filtration, affinity column chromatography, cation exchange chromatography, anion exchange chromatography, and concentration. The filtration step may comprise ultrafiltration, and optionally ultrafiltration and diafiltration. Filtration is preferably performed at least about 5-50 times, more preferably 10 to 30 times, and most preferably 14 to 27 times. Affinity column chromatography, may be performed using, for example, PROSEP® Affinity Chromatography (Millipore, Billerica, Mass.). In various embodiments, the affinity chromatography step comprises PROSEP®-vA column chromatography. Eluate may be washed in a solvent detergent. Cation exchange chromatography may include, for example, SP-Sepharose Cation Exchange Chromatography. Anion exchange chromatography may include, for example but not limited to, Q-Sepharose Fast Flow Anion Exchange. The anion exchange step is preferably non-binding, thereby allowing removal of contaminants including DNA and BSA. The antibody product is preferably nanofiltered, for example, using a Pall DV 20 Nanofilter. The antibody product may be concentrated, for example, using ultrafiltration and diafiltration. The method may further comprise a step of size exclusion chromatography to remove aggregates.

In some embodiments, different nucleic acid molecules encode a heavy chain variable region and a light chain variable region of a target specific antibody. In other embodiments, the same nucleic acid molecule encodes a heavy chain and a light chain variable regions of a target specific antibody. In one embodiment, the nucleic acid encodes a target specific antibody of the present disclosure, as well as any of the polypeptides encoded by the nucleic acids described herein.

In some embodiments, the nucleic acid molecule encodes a VH amino acid sequence that is at least 70%, 75%, 80%, 85%, 90%, 95%, 96%, 97%, 98% or 99% identical to a VH amino acid sequence set out in SEQ ID NOs: 22, 42, 62, 82, 102. 122, 142, 162, 182, 202, 222, 242, 262, 282, and 540. Nucleic acid molecules of the disclosure further include nucleic acids that hybridize under highly stringent conditions, such as those described herein, to a nucleic acid sequence encoding the heavy chain variable region amino acid sequence of SEQ ID NOs: 22, 42, 62, 82, 102. 122, 142, 162, 182, 202, 222, 242, 262, 282, and 540 or that has the heavy chain variable region nucleic acid sequence of any one of SEQ ID NOs: 20, 40, 60, 80, 100, 120, 140, 160, 180, 200, 220, 240, 260, 280, and 538.

In some embodiments, the nucleic acid molecule encodes a VL amino acid sequence that is at least 70%, 75%, 80%, 85%, 90%, 95%, 96%, 97%, 98% or 99% identical to a VL amino acid sequence set out in SEQ ID NOs: 21, 41, 61, 81, 101, 121, 141, 161, 181, 201, 221, 241, 261, 281, and 539. Nucleic acid molecules of the disclosure further include nucleic acids that hybridize under highly stringent conditions, such as those described herein, to a nucleic acid sequence encoding the light chain variable region amino acid sequence of SEQ ID NOs: 21, 41, 61, 81, 101, 121,141, 161, 181, 201, 221, 241, 261, 281, and 539, or that has the light chain variable region nucleic acid sequence of any one of SEQ ID NOs: 19, 39, 59, 79, 99, 119, 139, 159, 179, 199, 219, 239, 259, 279, and 537.

In one aspect, a nucleic acid molecule contemplated herein comprises a nucleotide sequence that encodes the VL amino acid sequence of anti-TREM-1 antigen binding proteins set out in herein or a portion thereof. In a related aspect, the VL amino acid sequence is a consensus sequence. In some embodiments, the nucleic acid encodes the amino acid sequence of the light chain CDRs of said antibody. In some embodiments, said portion is a contiguous portion comprising LCDR1-CDR3. In a related aspect, the LCDR1-3 amino acid sequences are consensus sequences. In one embodiment, said portion comprises at least one, two or three of a light chain CDR1, CDR2, or CDR3 region, optionally with a different human or human consensus framework, and optionally with 1, or up to 2, or up to 3 mutations in the collective 3 CDRs.

In one aspect, a nucleic acid molecule of the present disclosure comprises a nucleotide sequence that encodes the VH amino acid sequence of anti-TREM-1 antigen binding proteins set out herein, or a portion thereof. In a related aspect, the VH amino acid sequence is a consensus sequence. In some embodiments, the nucleic acid encodes the amino acid sequence of the heavy chain CDRs of said antibody. In some embodiments, said portion is a contiguous portion comprising HCDR1-CDR3. In a related aspect, the HCDR1-3 amino acid sequences are consensus sequences. In one embodiment, said portion comprises at least one, two or three of a heavy chain CDR1, CDR2, or CDR3 region, optionally with a different human or human consensus framework, and optionally with 1, or up to 2, or up to 3 mutations in the collective 3 CDRs.

In exemplary embodiments, an antibody of the disclosure comprises a human kappa (κ) or a human lambda (λ) light chain or an amino acid sequence derived therefrom, or a human heavy chain or a sequence derived therefrom, or both heavy and light chains together in a single chain, dimeric, tetrameric or other form.

### Derivatives

Suitable detectable molecules may be directly or indirectly attached to the antigen binding proteins of the present disclosure. Suitable detectable molecules include radionuclides, enzymes, substrates, cofactors, inhibitors, fluorescent markers, chemiluminescent markers, magnetic particles and the like. For indirect attachment of a detectable or cytotoxic molecule, the detectable or cytotoxic molecule can be conjugated with a member of a complementary/anti-complementary pair, where the other member is bound to the binding polypeptide or antibody portion. For these purposes, biotin/streptavidin is an exemplary complementary/anti-complementary pair.

The antigen binding proteins of the disclosure also include derivatives that are modified, e.g., by the covalent attachment of any type of molecule to the antibody such that covalent attachment does not prevent the antibody from binding to its epitope. Examples of suitable derivatives include but are not limited to derivatives that are fucosylated, glycosylated, acetylated, PEGylated, phosphorylated, or amidated. The, antigen binding proteins of the disclosure may themselves by derivatized by known protecting/blocking groups, proteolytic cleavage, linkage to a cellular ligand or other proteins, and the like. In some embodiments of the disclosure, at least one heavy chain of the antigen binding protein is PEGylated. In some embodiments, the PEGylation is N-linked or is linked through the sidechain of an amino acid (e.g., lysine).

Glycosylation can contribute to the effector function of antibodies, particularly IgG1 antibodies. Thus, in some embodiments, the antigen binding proteinsor of the disclosure may comprise one or more amino acid substitutions that affect the level or type of glycosylation of the binding proteins. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

In certain embodiments, glycosylation of the antigen binding proteins described herein is increased by adding one or more glycosylation sites, e.g., to the Fc region of the binding protein. Addition of glycosylation sites to the antigen binding protein can be conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence may be altered through changes at the DNA level, particularly by mutating the DNA encoding the target polypeptide at preselected bases such that codons are generated that will translate into the desired amino acids.

The disclosure also encompasses production of antigen binding proteins with altered carbohydrate structure resulting in altered effector activity, including antigen binding proteins with absent or reduced fucosylation that exhibit improved ADCC activity. Various methods are known in the art to reduce or eliminate fucosylation. For example, ADCC effector activity is mediated by binding of the antibody molecule to the FcγRIII receptor, which has been shown to be dependent on the carbohydrate structure of the N-linked glycosylation at the N297 residue of the CH2 domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (see Yamane-Ohnuki et al., Biotechnol Bioeng. 87(5):614-22, 2004). Similar effects can be accomplished through decreasing the activity of alpha-1,6-fucosyl transferase enzyme or other enzymes in the fucosylation pathway, e.g., through siRNA or antisense RNA treatment, engineering cell lines to knockout the enzyme(s), or culturing with selective glycosylation inhibitors (see Rothman et al., Mol Immunol. 26(12):1113-23, 1989). Some host cell strains, e.g. Lec13 or rat hybridoma YB2/0 cell line naturally produce antibodies with lower fucosylation levels (see Shields et al., J Biol Chem. 277(30):26733-40, 2002 and Shinkawa et al., J Biol Chem. 278(5):3466-73, 2003). An increase in the level of bisected carbohydrate, e.g. through recombinantly producing antibody in cells that overexpress GnTIII enzyme, has also been determined to increase ADCC activity (see Umana et al., Nat Biotechnol. 17(2):176-80, 1999).

In other embodiments, glycosylation of the antigen binding proteins described herein is decreased or eliminated by removing one or more glycosylation sites, e.g., from the Fc region of the binding protein. Amino acid substitutions that eliminate or alter N-linked glycosylation sites can reduce or eliminate N-linked glycosylation of the antigen binding protein. In certain embodiments, the bispecific antigen binding proteins described herein comprise a mutation at position N297 (EU numbering), such as N297Q, N297A, or N297G. In one particular embodiment, the bispecific antigen binding proteins of the invention comprise a Fc region from a human IgG1 antibody with a N297G mutation. To improve the stability of molecules comprising a N297 mutation, the Fc region of the molecules may be further engineered. For instance, in some embodiments, one or more amino acids in the Fc region are substituted with cysteine to promote disulfide bond formation in the dimeric state. Residues corresponding to V259, A287, R292, V302, L306, V323, or 1332 (EU numbering) of an IgG1 Fc region may thus be substituted with cysteine. In one embodiment, specific pairs of residues are substituted with cysteine such that they preferentially form a disulfide bond with each other, thus limiting or preventing disulfide bond scrambling. In certain embodiments pairs include, but are not limited to, A287C and L306C, V259C and L306C, R292C and V302C, and V323C and 1332C. In particular embodiments, the bispecific antigen binding proteins described herein comprise a Fc region from a human IgG1 antibody with mutations at R292C and V302C. In such embodiments, the Fc region may also comprise a N297G mutation.

Modifications of the antigen binding proteins of the disclosure to increase serum half-life also may desirable, for example, by incorporation of or addition of a salvage receptor binding epitope (e.g., by mutation of the appropriate region or by incorporating the epitope into a peptide tag that is then fused to the antigen binding proteins at either end or in the middle, e.g., by DNA or peptide synthesis; see, e.g., WO96/32478) or adding molecules such as PEG or other water soluble polymers, including polysaccharide polymers. The salvage receptor binding epitope preferably constitutes a region wherein any one or more amino acid residues from one or two loops of a Fc region are transferred to an analogous position in the antigen binding protein. In one embodiment, three or more residues from one or two loops of the Fc region are transferred. In one embodiment, the epitope is taken from the CH2 domain of the Fc region (e.g., an IgG Fc region) and transferred to the CH1, CH3, or VH region, or more than one such region, of the antigen binding protein. Alternatively, the epitope is taken from the CH2 domain of the Fc region and transferred to the CL region or VL region, or both, of the antigen binding protein. See International applications WO 97/34631 and WO 96/32478 for a description of Fc variants and their interaction with the salvage receptor.

The antigen binding proteins include variants having single or multiple amino acid substitutions, deletions, additions, or replacements that retain their biological properties. A person of ordinary skill in the art can produce variants having single or multiple amino acid substitutions, deletions, additions or replacements. These variants may include, inter alia: (a) variants in which one or more amino acid residues are substituted with conservative or non-conservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Antibodies and bispecific antibodies of the invention may include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques, are known to the person having ordinary skill in the art.

### Methods of Treatment

TREM-1 is implicated in cardiovascular indications. TREM-1 has been shown to be expressed in human atheromas and is upregulated under dyslipidemic conditions on circulating and on lesion-infiltrating myeloid cells in the Apoe−/− mouse model. It was demonstrated that Trem1^{-/-}Apoe^{-/-} mice exhibit attenuated diet-induced atherogenesis. (Zysset et al., Nat. Commun. 7:1-16, 2016). Boufenzer et al. (Circ. Res. 2015, 116:1772-1782) demonstrated that TREM1 Deficiency inhibits recruitment and activation of inflammatory cells to the infarcted myocardium, identifying that inhibition of TREM-1 using short peptides or gene knockout reduces myocardial infiltration of neutrophils, and improves heart function and survival after MI in mice and rats. In humans, TREM-1 activation, as evidenced by increased levels of sTREM-1, after MI is a predictor of mortality (Boufenzer et al., supra).

Lui et al. (Nat. Immunol 20:1023-1034., 2019) showed that peripheral myeloid cells magnify stroke injury via activation of TREM1. Neutrophils and macrophages infiltrating the brain highly express Trem1-activated by immunogenic cellular components, e.g., danger/damage-associated molecular patterns (DAMPs) (MCAo mouse model) (Colonna, M. Trends in Immun. 2019, 40: 781-783). Trem1 inhibition by decoy peptides or Trem-1 knockout exhibited decreased infiltration of neutrophils/macrophages into the site of injury, smaller infarct volumes, and improved neurological scores and increased survival (Lui et al, supra; Colonna, supra).

TREM-1 is also implicated in development of NAFLD/NASH. Rao et al., (J Cell Biochem 120:11867-11877, 2019) showed that overexpression of Trem1 in mouse hepatocytes resulted in increased lipid accumulation and genes involved in lipid uptake (e.g. Id/r, msr1) were upregulated when Trem1 was overexpressed. Additionally, genes involved in cholesterol efflux (e.g., abca1, abcg1) were downregulated when Trem1 was overexpressed. Oleic acid treatment of hepatocytes (HepG2, PMH) overexpressing Trem1 resulted in increased expression of inflammatory cytokines. Knockdown of TREM-1 using shRNA reduced lipid accumulation in mice on a high fat diet.

Obesity is characterized by chronic systemic inflammation (higher TNFα, IL6, and hsCRP), and TREM-1 has been shown to be increased in obese patients. It is hypothesized herein that therapy with anti-TREM1, optionally in combination with anti-obesity drugs, would lead to a greater reduction in systemic inflammation.

It is contemplated herein that antigen binding proteins specific for TREM-1 disclosed herein are useful to treat cardiovascular diseases. Exemplary cardiovascular diseases include myocardial infarction, non-alcoholic fatty liver disease (NAFLD), non-alcoholic steatohepatitis (NASH), heart failure, stroke (ischemic and hemorrhagic), atherosclerosis, coronary artery disease, peripheral vascular disease (e.g. peripheral artery disease), vulnerable plaque, acute coronary syndrome, cerebrovascular disease, cerebrovascular atherosclerosis and obesity.

In certain embodiments, measurement of TNF-α suppression is carried out using isolated PBMC. PBMC are isolated from blood of a subject (human, mouse, rat, cynomolgus monkey, and the like), stimulated in vitro with LPS or TREM-1 ligand (Peptidoglycan recognition protein complexed with bacterial peptidoglycan) and levels of TNFα before and after stimulation, and in the presence of different test molecules, are determined using, for examples an ELISA, ALPHALISA or MSD Vplex TNFα detection kit.

In one embodiment, the disclosure provides a method of inhibiting one or more of proinflammatory cytokines, e.g., TNFα, in a mammal in need of such treatment comprising administering a therapeutically effective amount of an antigen binding protein or antibody of the disclosure, to a subject in need of such treatment. In a preferred embodiment, the subject is a mammal. In one embodiment, the subject is a human. The method may be used to treat a disorder characterized by elevated expression or activity of TNFα. The antigen binding protein or antibody of the disclosure, may be administered with another pharmaceutical agent, either in the same formulation or separately.

Additional assays contemplated include measuring the concentration of high sensitivity C-reactive protein, concentration of soluble TREM-1, ex-vivo analysis of phosphorylated spleen tyrosine kinase in isolated PBMCs, and analysis of myeloid cell subsets.

It is contemplated that the treatment reduces the levels of inflammatory cytokines such as TNF-α and IL-6 in the subject.

It is contemplated that treatment with an anti-TREM-1 antigen binding protein of the disclosure comprising an anti-TREM-1 binding protein reduces expression of TREM-1 in inflammatory cells.

### Administration and Dosing

Methods of the present disclosure include a step of administering a pharmaceutical composition comprising an antigen binding protein described herein. In certain embodiments, the pharmaceutical composition is a sterile composition.

The amounts of therapeutic composition in a given dosage may vary according to the size of the individual to whom the therapy is being administered as well as the characteristics of the disorder being treated.

The present disclosure provides a composition comprising an antigen binding protein or antibody of the disclosure and a pharmaceutically acceptable carrier. A pharmaceutical composition comprising an antigen binding protein or antibody of the disclosure can be formulated according to known methods to prepare pharmaceutically useful compositions, whereby the therapeutic antibodies are combined in a mixture with a pharmaceutically acceptable carrier. A composition is said to comprise a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Getman), ed., Remington's Pharmaceutical Sciences, 19th Edition, Mack Publishing Company (1995).

For pharmaceutical use, polypeptides of the present disclosure are formulated for parenteral, particularly intravenous or subcutaneous, delivery according to conventional methods. Intravenous administration may be by bolus injection, controlled release, e.g., using mini-pumps or other appropriate technology, or by infusion over a typical period of one to several hours. In general, pharmaceutical formulations will include an antigen binding protein or antibody of the disclosure, in combination with a pharmaceutically acceptable carrier, such as saline, buffered saline, 5% dextrose in water or the like. Formulations may further include one or more excipients, preservatives, solubilizers, buffering agents, albumin to prevent protein loss on vial surfaces, etc. When utilizing such a combination therapy, antigen binding protein or antibody of the disclosure, may be combined in a single formulation or may be administered in separate formulations. Methods of formulation are well known in the art and are disclosed, for example, in Gennaro, ed., Remington's Pharmaceutical Sciences, Mack Publishing Co., Easton Pa. (1990), which is incorporated herein by reference. Therapeutic doses will generally be in the range of 0.1 to 100 mg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. More commonly, the antibodies will be administered over one week or less, often over a period of one to three days. Generally, the dosage of administered antibodies will vary depending upon such factors as the patient's age, weight, height, sex, general medical condition and previous medical history. Typically, it is desirable to provide the recipient with a dosage of antibodies which is in the range of from about 1 pg/kg to 10 mg/kg (amount of agent/body weight of patient), although a lower or higher dosage also may be administered as circumstances dictate.

Administration of an antigen binding protein or antibody of the disclosure, to a subject can be intravenous, intraarterial, intraperitoneal, intramuscular, subcutaneous, intrapleural, intrathecal, by perfusion through a regional catheter, or by direct intralesional injection. In various embodiments the administration is intravenous or subcutaneous. When administering antigen binding proteins by injection, the administration may be by continuous infusion or by single or multiple boluses.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase et al., “Oral Delivery of Microencapsulated Proteins”, in Sanders et al., eds., Protein Delivery: Physical Systems, pp. 255-288, Plenum Press (1997)). The feasibility of an intranasal delivery is exemplified by such a mode of insulin administration (see, for example, Hinchcliffe et al., Adv. Drug Deliv. Rev., 35:199 (1999)). Dry or liquid particles comprising antibodies of the invention can be prepared and inhaled with the aid of dry-powder dispersers, liquid aerosol generators, or nebulizers (e.g., Pettit et al., TIBTECH, 16:343 (1998); Patton et al., Adv. Drug Deliv. Rev., 35:235 (1999)). This approach is illustrated by the AERX® diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of transcutaneous administration (Mitragotri et al., Science, 269:850 (1995)).

For purposes of therapy, compositions comprising an antigen binding protein or antibody of the disclosure and a pharmaceutically acceptable carrier are administered to a patient in a therapeutically effective amount. A combination of antigen binding protein or antibody of the disclosure and a pharmaceutically acceptable carrier is said to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response. Effective treatment may be assessed in a variety of ways. In one embodiment, effective treatment is determined by reduced inflammation. In other embodiments, effective treatment is marked by inhibition of inflammation. In still other embodiments, effective therapy is measured by increased well-being of the patient including such signs as weight gain, regained strength, decreased pain, thriving, and subjective indications from the patient of better health.

It is contemplated that the antigen binding protein, e.g., comprising anti-TREM-1 antibody sequences, is administered at a dose of 30 mg, 40 mg, 50 mg, 60 mg, 70 mg, 80 mg, 90 mg, 100 mg, 125 mg, 150 mg, 200 mg, 250 mg, 300 mg or more. In various embodiments, antigen binding protein, e.g., comprising anti-TREM-1 antibody sequences, is administered at a dose of about 0.05 mg/kg, 0.25 mg/kg, 0.5 mg/kg, 0.75 mg/kg, 1.0 mg/kg, 1.25 mg/kg, 1.5 mg/kg, 2.0 mg/kg, 2.5 mg/kg, 3.0 mg/kg, 3.5 mg/kg, 4.0 mg/kg, 4.5 mg/kg or 5 mg/kg.

Compositions described herein are administered once weekly, twice weekly, once every two weeks, once every three weeks, once every 4 weeks, once monthly, once every 3 months, once every six months, or once yearly.

A pharmaceutical composition comprising an antigen binding protein or antibody of the disclosure can be furnished in liquid form, in an aerosol, or in solid form. Liquid forms, are illustrated by injectable solutions and oral suspensions. Exemplary solid forms include capsules, tablets, and controlled-release forms. The latter form is illustrated by miniosmotic pumps and implants (Bremer et al., Pharm. Biotechnol., 10:239 (1997); Ranade, “Implants in Drug Delivery”, in Ranade et al., eds., Drug Delivery Systems, pp. 95-123, CRC Press (1995); Bremer et al., “Protein Delivery with Infusion Pumps”, in Sanders et al., eds., Protein Delivery: Physical Systems, pp. 239-254, Plenum Press (1997); Yewey et al., “Delivery of Proteins from a Controlled Release Injectable Implant”, in Sanders et al., eds., Protein Delivery: Physical Systems, pp. 93-117, Plenum Press (1997).

The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In one embodiment, an antigen binding protein or antibody of the disclosure is administered in combination therapy, i.e., combined with other agents, e.g., therapeutic agents, that are useful for treating pathological conditions or disorders, such as cardiovascular diseases. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

It is contemplated the therapeutic agents of the present disclosure may be given simultaneously, in the same formulation. It is further contemplated that the agents are administered in a separate formulation and administered concurrently, with concurrently referring to agents given within 30 minutes of each other. It is further contemplated that a second agent may be given simultaneously.

In another aspect, an antigen binding protein as described herein is administered prior to administration of a second composition. Prior administration refers to administration of an agent within the range of one week prior to treatment with the other agent, up to 30 minutes before administration of the other agent. It is further contemplated that an agent is administered subsequent to administration of another composition or agent. Subsequent administration is meant to describe administration from 30 minutes after antibody treatment up to one week after antibody administration, e.g., 30 minutes, 1 hour 2 hours, 4 hours, 1 day, 2 days, etc. It is further contemplated that a second

For example, the combination therapy can include one or more antigen binding protein or antibody of the disclosure co-formulated with, and/or co-administered with, one or more additional therapeutic agents, e.g., one or more cholesterol-lowering (serum and/or total body cholesterol) agent, an agent that increases the expression of LDLR, statins (atorvastatin, cerivastatin, fluvastatin, lovastatin, mevastatin, pitavastatin, pravastatin, rosuvastatin, simvastatin), PCSK9 inhibitors (Repatha®, Praluent®, Leqvio®) nicotinic acid (Niacin) (NIACOR®, NIASPANG (slow release niacin), SLO-NIACIN® (slow release niacin), Fibric acid (LOPID® (gemfibrozil), TRICOR® (fenofibrate), Bile acid sequestrants (QUESTRAN® (cholestyramine), colesevelam (WELCHOL®), COLESTID® (colestipol), Cholesterol absorption inhibitors (Zetia (ezetimibe)), combining nicotinic acid with statin (ADVICOR® (LOVASTATIN and NIASPAN®), combination of a statin with an absorption inhibitor (VYTORIN (ZOCOR® and ZETIA®) and/or lipid modifying agents like PCSK9 inhibitors, PPAR gamma agonists, PPAR alpha/gamma agonists, squalene synthase inhibitors, cholesterylester transfer protein (CETP) inhibitors, anti-hypertensives, anti-thrombotics (aspirin) anti-diabetic agents (such as sulphonyl ureas, insulin, GLP-1 analogs, DDPIV inhibitors, SGL2 inhibitors), ApoB modulators, MTP inhibitors, Corlanorg (ivabradine) I(f) current Inhibitor, omecamtiv mecarbil cardiac myosin activator, OLPASIRAN (AMG890) (siRNA) that lowers lipoprotein(a), AMG 594 cardiac troponin activator, AMG 609, AMG 171 (Growth Differential Factor 15 (GDF15) analog), AMG 133 (gastric inhibitory polypeptide receptor (GIPR) antagonist and glucagon-like peptide 1 (GLP-1) receptor agonist), and/or arteriosclerosis obliterans treatments.

Therapeutic agents used in combination with an antigen binding protein or antibody of the disclosure include agents that interfere at different stages in an inflammatory response. In one embodiment, an antigen binding protein or antibody of the disclosure, may be co-formulated with, and/or co-administered with, one or more additional agents such as other cytokine or growth factor antagonists (e.g., soluble receptors, peptide inhibitors, small molecules, ligand fusions); or antibodies or antigen binding fragments thereof that bind to other targets (e.g., antibodies that bind to other cytokines or growth factors, their receptors, or other cell surface molecules); and anti-inflammatory cytokines or agonists thereof. Non-limiting examples of the agents that can be used in combination with the antibodies described herein, include, but are not limited to, antagonists of one or more interleukins (ILs) or their receptors, e.g., antagonists of IL-1, IL-2, IL-6, IL-7, IL-8, IL-12, IL-13, IL-15, IL-16, IL17A-F, IL-18, IL-20, IL-21, IL-22, IL-23 IL-25, IL-31, IL-32, IL-33; antagonists of cytokines or growth factors or their receptors, such as, LT, EMAP-II, GM-CSF, FGF and PDGF. Antibodies of the invention can also be combined with inhibitors of e.g., antibodies to, cell surface molecules such as CD2, CD3, CD4, CD8, CD20 (e.g., the CD20 inhibitor rituximab (RITUXAN®), CD25, CD28, CD30, CD40, CD45, CD69, CD80 (B7.1), CD86 (B7.2), CD90, or their ligands, including CD154 (gp39 or CD40L), or LFA-1/ICAM-1 and VLA-4/VCAM-1 (Yusuf-Makagiansar et al., Med. Res. Rev., 22:146-167 (2002)). Exemplary antagonists that can be used in combination include antagonists of IL-1, IL-6, IL-12, TNFα, IL-15, IL-18, IL-20, IL-22, IL-23 and IL-31.

In other embodiments, one or more antigen binding proteins of the disclosure can be co-formulated with, and/or co-administered with, one or more anti-inflammatory drugs, immunosuppressants, or metabolic or enzymatic inhibitors. Non-limiting examples of the drugs or inhibitors that can be used in combination with the antibodies described herein, include, but are not limited to, one or more of: nonsteroidal anti-inflammatory drug(s) (NSAIDs), e.g., ibuprofen, tenidap, naproxen, meloxicam, piroxicam, diclofenac, and indomethacin; sulfasalazine; corticosteroids such. as prednisolone; cytokine suppressive anti-inflammatory drug(s) (CSAIDs); inhibitors of nucleotide biosynthesis, e.g., inhibitors of purine biosynthesis, folate antagonists (e.g., methotrexate (N-[4-[[(2,4-diamino-6-pteridinyl)methyl]methylamino]benzoyl]-glutamic acid); and inhibitors of pyrimidine biosynthesis, e.g., dihydroorotate dehydrogenase (DHODH) inhibitors. Preferred therapeutic agents for use in combination with one or more antibodies, e.g., bispecific antibodies, of the invention include NSAIDs, CSAIDs, (DHODH) inhibitors (e.g., leflunomide), and folate antagonists (e.g., methotrexate.

Additional inhibitors include one or more of: corticosteroids (oral, inhaled and local injection); immunosuppressants, e.g., cyclosporin, tacrolimus (FK-506); and mTOR inhibitors, e.g., sirolimus (rapamycin—RAPAMUNE® or rapamycin derivatives, e.g., soluble rapamycin derivatives (e.g., ester rapamycin derivatives, e.g., CCI-779); agents which interfere with signaling by proinflammatory cytokines such as IL-1 (e.g., IRAK, NIK, IKK, p38 or MAP kinase inhibitors); COX2 inhibitors, e.g., celecoxib, rofecoxib, and variants thereof; phosphodiesterase inhibitors, e.g., R973401 (phosphodiesterase Type IV inhibitor); phospholipase inhibitors, e.g., inhibitors of cytosolic phospholipase 2 (cPLA2) (e.g., trifluoromethyl ketone analogs); inhibitors of vascular endothelial cell growth factor or growth factor receptor, e.g., VEGF inhibitor and/or VEGF-R inhibitor; and inhibitors of angiogenesis. Preferred therapeutic agents for use in combination with an antigen binding protein or antibody of the disclosure are immunosuppressants, e.g., cyclosporin, tacrolimus (FK-506); mTOR inhibitors, e.g., sirolimus (rapamycin) or rapamycin derivatives, e.g., soluble rapamycin derivatives (e.g., ester rapamycin derivatives, e.g., CCI-779); COX2 inhibitors, e.g., celecoxib and variants thereof; and phospholipase inhibitors, e.g., inhibitors of cytosolic phospholipase 2 (cPLA2), e.g., trifluoromethyl ketone analogs.

### Kits

The disclosure also contemplates a kit comprising one or more containers that comprises an antigen binding protein or antibody of the disclosure, optimally in a pharmaceutically acceptable carrier or composition. The antigen binding protein or antibody of the disclosure can be provided in the form of an injectable solution for single or multiple doses, as a unit dose, or as a sterile powder that will be reconstituted before injection. Alternatively, such a kit can include a dry-powder disperser, liquid aerosol generator, or nebulizer for administration of the therapeutic agent(s). Such a kit may further comprise instructions and written information on indications and usage of the pharmaceutical composition.

Syringes, e.g., single use or pre-filled syringes, sterile sealed containers, e.g. vials, bottle, vessel, and/or kits or packages comprising any of the foregoing antigen binding proteins or compositions, optionally with suitable instructions for use, are also contemplated.

In a further embodiment, the invention provides an article of manufacture, or unit dose form, comprising: (a) a composition of matter comprising an antigen binding protein or antibody of the disclosure; (b) a container containing said composition; and (c) a label affixed to said container, or a package insert included in said container referring to the use of said antibody in the treatment of an immune related disease.

In another aspect, the composition or kit comprises a further active ingredient, which may, for example, be a further antibody or an anti-inflammatory, cytotoxic or other agent described herein. Preferably, the composition is sterile.

### Examples

### Example 1-Anti-TREM-1 Antibodies

High affinity anti-TREM-1 monoclonal antibodies were generated and assessed for the ability to target to immune cells and modulate signaling between TREM-1 and its ligand(s).

Fully human antibodies to human TREM-1 were generated by immunizing XENOMOUSE® transgenic mice. See for example, U.S. Pat. Nos. 6,114,598; 6,162,963; 6,833,268; 7,049,426; and 7,064,244.

Mice were immunized with human and/or cyno TREM1 protein, TREM1 expression vectors and/or TREM1 expressing CHO cells. For genetic immunizations, mice were immunized 16 times over 8 weeks using the HELIOS® Gene Gun system according to the manufacturer's instructions (BioRad, Hercules, California). Briefly, expression vectors encoding either human TREM-1 and DAP12, or cyno TREM-1 and DAP12, were pooled and 2 pg total DNA were coated onto 1.6 um gold beads (BioRad, Hercules, California) and delivered to the epidermis of a shaved mouse abdomen. For soluble protein immunizations, mice were immunized with human or cyno TREM-1 recombinant protein representing the N-terminal extracellular domain. Animals were immunized with recombinant protein adjuvanted with either Alum and CpG-ODN or Sigma Adjuvant System, 14-17 times over 10-12 weeks using subcutaneous injections delivered at two locations along the dorsal midline of the mice located at the base of tail and subscapular region. The initial soluble protein immunization delivered 10 pg and subsequent boosts were 5 pg. For cell immunizations, mice were immunized with 2-4 million CHO-S cells transiently expressing either human TREM-1 or cyno TREM-1 adjuvanted with Alum and CpG-ODN. Animals were immunized 1 or 2 times weekly for a total of 13 times over 10 weeks alternating between intraperitoneal and subcutaneous injections at the base of tail. Animals were bled, and plasma collected at various time points during the immunization studies ranging from 4 weeks to 10 weeks to assess for TREM1-specific titers. TREM1-specific plasma titers were monitored by live-cell FACS analysis on an ACCURI™ flow cytometer (BD Biosciences), using transiently transfected 293T cells. Animals with the highest antigen-specific plasma titers against human and cyno TREM1 were sacrificed and used for hybridoma generation (Kohler and Milstein, 1975)

Hybridoma Generation: Animals exhibiting suitable antigen-specific serum titers were identified and spleen and/or draining lymph nodes from select mice were pooled from each harvest. Splenocytes and lymphocytes were dissociated from lymphoid tissue by grinding in a suitable medium or using the GENTLEMACS™ Dissociator (Miltenyi Biotec) semi-automated tissue dissociation instrument. IgG-expressing B cells were isolated, expanded using standard methods, and fused with a suitable cell fusion partner. Hybridoma supernatants were tested for binding to human TREM-1 transiently expressed on HEK293 cells by CELLINSIGHT™. Briefly, HEK293 cells were transiently co-transfected with a 1:1 ratio of mammalian expression constructs encoding human TREM-1 and DAP12, or mock vector and DAP12 alone using 293Fectin (Invitrogen) following the manufacturer's protocol. The following day, 15,000 cells/well of transfected HEK293 cells were combined with an equal volume of exhausted hybridoma media test samples and nuclear stain Hoechst 33342 (Pierce) at 15 pg/mL final concentration, at a total volume of 30 μL/well in 384-well FMAT plates (Corning). After 1 hour incubation at room temperature, the supernatant was aspirated using an AQUAMAX® plate washer, and wells were washed for 2 cycles using 50 pL/well of FACS buffer (PBS (Hyclone), 2% FBS (Sigma)) each cycle on the AquaMax. Cells were stained with 5 Pg/mL Alexa Fluor 488 Goat anti-Human IgG Fc (Jackson ImmunoResearch) secondary antibody, shaken on a Big Bear plate shaker, and incubated at room temperature for 20 minutes. The supernatant was aspirated using an AQUAMAX® plate washer, wells were again washed for 2 cycles using 50 pL/well of FACS buffer, and 30 μL of FACS buffer was added to each well using a multidrop instrument. The plates were placed on a Big Bear Plate shaker to evenly distribute the cells in the wells, and then read on the CELLINSIGHT™CX7 platform using the Cell Health Profiling Bio-App.

The TREM-1-specific antibodies identified in primary screening were evaluated for cross-reactivity to cynomolgus TREM-1, as well as specificity to TREM-1 and not DAP12. TREM-1 hybridoma supernatants were tested for binding to human or cyno TREM-1 transiently expressed on HEK293 cells by FACS (harvests 1-6) or by CELLINSIGHT™ (harvests 8-9). For TREM1 antibodies from harvests 1-6, HEK293 cells were transiently co-transfected with a 1:1 ratio of mammalian expression constructs encoding human TREM-1 and DAP12, cyno TREM-1 and DAP12, or mock vector and human DAP12 using 293Fectin. The following day, transfected HEK293 cells were transferred into 96-well FACS plates at 50,000 cells/well and incubated with normalized hybridoma supernatants at a final concentration of 2.5 μg/mL for 1 hour at 4° C. Cells were then pelleted by centrifugation, supernatant was removed by flicking, and wells were washed twice with 200 μL/well of FACS buffer. 5 μg/mL ALEXA FLUOR® 647 Goat anti-Human IgG Fc (Jackson ImmunoResearch) secondary detection antibody and 2.5 μg/mL 7-aminoactinomycin-D (Sigma) viability stain were incubated with the cells for 15 minutes at 4° C. Cells were pelleted by centrifugation, supernatant was removed by flicking, and wells were washed once more with 200 μL/well of FACS buffer. TREM-1 hybridoma supernatants showing human TREM-1- or cyno TREM-1-specific binding was detected by FACS on the BD ACCURI™ 06 flow cytometer with Intellicyt autosampler. The data was reported as geomean (GM) fold over irrelevant control antibody binding. Results of binding for certain anti-TREM-1 antibodies is shown in Table 1.

TABLE 1
TREM1 antibodies in human/cyno cross-
reactivity and specificity screen

| Antibody ID | Human TREM1 GM Fold | Cyno TREM1 GM Fold |
|---|---|---|
| 3E12 | 279.6 | 176.5 |
| 34D1 | 245.8 | 228.1 |
| 30H2 | 108.1 | 76.9 |
| 44A5 | 1634.6 | 2238.4 |
| 46H7 | 1907.9 | 2688.8 |
| 49A2 | 2218.0 | 2978.4 |
| 50A12 | 2421.0 | 2782.1 |
| 57C10 | 1667.8 | 2429.0 |
| 57F5 | 718.8 | 2338.9 |
| 61B12 | 1850.3 | 2093.4 |
| 61G5 | 2072.8 | 2160.6 |
| 63F8 | 1384.3 | 2372.3 |
| 64D7 | 1476.6 | 2151.3 |
| 66B8 | 1634.0 | 1517.9 |

The TREM1 antibodies demonstrating high quality TREM1-specific binding and human/cyno cross-reactivity were evaluated for their ability to block ligand PGLYRP1 from binding to human TREM1/DAP12 transiently expressed on HEK293 cells. Briefly, HEK293 cells were transiently co-transfected with a 1:1 ratio of mammalian expression constructs encoding human TREM1 and DAP12, or mock vector and human DAP12 using 293Fectin. The following day, transfected HEK293 cells were transferred into 96-well FACS plates at 50,000 cells/well and incubated with normalized hybridoma supernatants at a final concentration of 2.5 μg/mL for 1 hour at 4° C. Human PGLYRP1-His (R&D Systems) was combined with PGN-ECndss (peptidoglycan, InvivoGen) and incubated at room temperature for 15 minutes, then added to the wells at a final concentration of 7.5 μg/mL PGLYRP1 and 30 μg/mL PGN. The plates were then shaken and incubated for 15 minutes at 4° C. Cells were then pelleted by centrifugation, supernatant was removed by flicking, and wells were washed with 200 μL of FACS buffer. 5 μg/mL ALEXA FLUOR® 647 human anti-His secondary detection antibody and 2.5 μg/mL 7-aminoactinomycin-D (Sigma) viability stain were added to the cells, shaken, and incubated for 15 minutes at 4° C. Cells were washed with FACS buffer, pelleted by centrifugation, supernatant was removed by flicking, and washed once more with FACS buffer. Cells were then run on the BD ACCURI™C6 Flow Cytometer with Intellicyt HYPERCYT® autosampler. A total of 518 TREM1 antibodies showed the desired specific PGLYRP1-blocking activity. Receptor-ligand inhibition for select TREM-1 monoclonal antibodies are summarized in Table 2.

TABLE 2
Receptor-ligand inhibition for select TREM1 antibodies

| Antibody ID | Receptor-ligand % inhibition |
|---|---|
| 3E12 | 101% |
| 34D1 | 101% |
| 30H2 | 101% |
| 44A5 | 100% |
| 46H7 | 100% |
| 49A2 | 100% |
| 50A12 | 100% |
| 57C10 | 100% |
| 57F5 | 100% |
| 61B12 | 87% |
| 61G5 | 91% |
| 63F8 | 93% |
| 64D7 | 96% |
| 66B8 | 85% |

TREM-1 Antibody Relative Affinity Ranking by Limiting Antigen Assay: TREM1 hybridoma supernatants were affinity-ranked within the panel by their binding kinetics to soluble TREM-1 in a limiting antigen assay using LUMAVIDIN® beads (Luminex) on FACS. Briefly, in-house biotinylated human TREM-1-His antigen (b-huTREM-1-His) was serially diluted in FACS buffer and combined with an equal volume of LUMAVIDIN® beads (different uniquely-barcoded bead for each antigen concentration), resulting in a 5-point 2-fold serial dilution series starting from a final b-huTREM-1-His antigen concentration of 30 ng/mL. The antigen-bead mixtures were plated across 3 wells in a 96-well FACS plate, then incubated for 30 minutes at room temperature protected from light. Beads were then pelleted by centrifugation, supernatant was removed by flicking, and wells were washed twice with 200 μL/well of FACS buffer. The different beads were then resuspended, pooled, and diluted in STABILGUARD® Immunoassay Stabilizer (SurModics) to block non-specific binding. Normalized TREM-1 hybridoma supernatants were combined with an equal volume of bead mixture in a FACS plate at a final concentration of 5 μg/mL test antibody to 0.5 μL beads/well. The plates were then shaken and incubated overnight for ˜18 hours at room temperature. Beads were then pelleted by centrifugation, supernatant was removed by flicking, and wells were washed twice with 200 μL/well of FACS buffer. ALEXA FLUOR® 488 Goat anti-Human IgG Fc secondary detection antibody (Jackson ImmunoResearch) was added at 5 μg/mL to the plates, shaken, and incubated for 15 minutes at room temperature protected from light. Beads were washed with FACS buffer, pelleted by centrifugation, supernatant was removed by flicking, and washed once more with FACS buffer. The beads were then resuspended and run on the BD ACCURI™ C6 Flow Cytometer with Intellicyt HYPERCYT® autosampler.

TREM-1 hybridoma samples showing at least two times or greater signal over control IgG antibody samples were considered to be exhibiting TREM-1-specific binding profiles. The antibody binding signal correlates with antibody affinity; the degree of antibody binding to the target antigen TREM-1 correlates with the measured fluorescent intensity and thus allows a relative comparison of affinities across the panel. TREM-1 antibodies with better binding in limiting antigen screens than the benchmark antibody 1B2 were advanced to light chain sequencing and human/cyno affinity gap analysis. Table 3 shows the antibody binding data for select TREM-1 antibodies using a representative antigen coating concentration that fell within the linear range of the instrument signal detection

TABLE 3
Relative affinities of select TREM1 antibodies
in limited antigen binding assays

| Antibody ID | Soluble TREM1 7.5 ng/mL (FACS Geomean) |
|---|---|
| 3E12 | 121096 |
| 34D1 | 80444 |
| 30H2 | 91743 |
| 44A5 | 36759 |
| 46H7 | 37975 |
| 49A2 | 28315 |
| 50A12 | 14642 |
| 57C10 | 28498 |
| 57F5 | 25282 |
| 61B12 | 26854 |
| 61G5 | 24835 |
| 63F8 | 25759 |
| 64D7 | 24511 |
| 66B8 | 23784 |

TREM1 Relative Epitope Binning/Profiling: TREM1 hybridoma supernatants were assessed by epitope binning assay (a modified antibody-antibody competition assay) using LUMAVIDIN® beads (Luminex) on FACS to determine the variety of relative unique epitope bins in the panel. Briefly, a set of 15 different uniquely-barcoded LUMAVIDIN® beads were each combined with an equal volume of in-house biotinylated human TREM-1-His antigen diluted in FACS buffer at a final concentration of 100 ng/mL. The antigen-bead mixtures were plated across 3 wells in a 96-well FACS plate, then incubated for 30 minutes at room temperature protected from light. Beads were then pelleted by centrifugation, supernatant was removed by flicking, and wells were washed twice with 200 μL/well of FACS buffer. 15 different TREM-1 antibodies with diverse VDJ rearrangements and good quantitation that had shown good binding in the limiting antigen assay were chosen as reference antibodies for pre-coating the beads. These 15 antibodies were prepared at a saturating concentration of 5 μg/mL in FACS buffer and incubated with each of the 15 different LUMAVIDIN® beads for 1 hour at room temperature protected from light. Beads were pelleted by centrifugation, supernatant was removed by flicking, and wells were washed three times with 200 μL/well of FACS buffer. The different beads were then resuspended, pooled, and diluted in STABILGUARD® Immunoassay Stabilizer (SurModics) to block non-specific binding. Normalized TREM-1 hybridoma supernatants (test antibodies) were combined with an equal volume of bead mixture in a FACS plate at a final concentration of 5 μg/mL test antibody to 0.5 μL beads/well. The plates were then shaken and incubated for 1 hour at room temperature protected from light. Beads were then pelleted by centrifugation, supernatant was removed by flicking, and wells were washed twice with 200 μL/well of FACS buffer. ALEXA FLUOR® 488 Goat anti-Human IgG Fc secondary detection antibody (Jackson ImmunoResearch) was added at 5 μg/mL to the plates, shaken, and incubated for 15 minutes at room temperature protected from light. Beads were washed with FACS buffer, pelleted by centrifugation, supernatant was removed by flicking, and washed once more with FACS buffer. The beads were then resuspended and run on the BD ACCURI™ C6 Flow Cytometer with Intellicyt HYPERCYT® autosampler.

Test antibodies competing to a similar epitope on the TREM-1 antigen as the reference antibodies are prevented from binding while non-competing antibodies are able to bind generating an additive signal with the reference antibody. The total bound antibodies are then detected with the secondary antibody. To determine the antibody competition/binding profiles of the individual test antibodies, the reference-only antibody binding signal was subtracted from the reference plus test antibody signal for each competition/binding reaction (i.e., across the entire reference antibody set). A summary of the relative epitope binning for select TREM-1 antibodies is shown below in Table 4.

TABLE 4
Relative epitope bins for select top TREM1 antibodies

| Antibody ID | Epitope Bin |
|---|---|
| 3E12 | B |
| 34D1 | ND |
| 30H2 | ND |
| 44A5 | A |
| 46H7 | A |
| 49A2 | A |
| 50A12 | A.1 |
| 57C10 | A |
| 57F5 | A |
| 61B12 | A |
| 61G5 | A |
| 63F8 | A |
| 64D7 | A |
| 66B8 | A |

TREM1 Antibody Human/Cynomolgus Affinity Gap Determination: TREM-1 antibodies with unique CDR3 sequences that had also shown better binding in limiting antigen screens than the benchmark antibody were analyzed for their affinity to human and cyno TREM-1. The binding affinity K_{D} (M), association rate constant kₐ (M⁻¹s⁻¹⁻), and dissociation rate constant k_{d} (s⁻¹), were determined on a panel of 114 TREM-1 antibodies using the OCTET® HTX instrument (Fortebio). Briefly, TREM-1 hybridoma supernatants normalized to 10 μg/mL in DMEM null media were prepared by diluting 1:10 in OCTET® assay buffer (10 mM Tris, 0.1% Triton X-100, 150 mM NaCl, 1 mM CaCl₂, 0.1 mg/mL BSA, at pH 7.6) to a final test concentration of 1 μg/mL. Amine reactive second-generation AR2G Biosensors (Molecular Devices) were pre-incubated in 200 μL nanopore water for a minimum of 10 minutes at room temperature before use. The AR2G Biosensors were then activated for 5 minutes in a solution of 20 mM EDC (1-Ethyl-3-[3-dimethylaminopropyl] carbodiimide hydrochloride) (ForteBio) pre-mixed with 10 mM NHS (N-hydroxysulfosuccinimide) (ForteBio) in nanopore water. An in-house generated mouse anti-human Fc monoclonal antibody was coupled to the AR2G Biosensors at 10 μg/mL in 10 mM sodium acetate buffer at pH 5 for 5 minutes, quenched with 1M ethanolamine at pH 8.5 for 5 minutes, and then used to capture antibody from solution. The TREM-1 test antibodies were loaded onto the Biosensors for 5 minutes, and baseline measurements were taken for 1 minute. The recombinant soluble human TREM-1-His protein was then bound to the antibody-loaded Biosensors in a 3-fold dilution series covering 6 points from 450 nM to 1.85 nM or 150 nM to 0.62 nM. The association of recombinant human TREM-1 with the antibody-loaded sensor was measured for 5 minutes, followed by dissociation in OCTET® buffer for 10 minutes. Biosensors were then regenerated with 10 mM glycine at pH 1.7, reloaded with the same TREM-1 antibodies on the same sensors for 5 minutes, and the same method was used to measure association and dissociation of cyno TREM-1-His protein. Data was referenced using a 0 nM analyte reference sensor. Kinetic analysis was performed using a 1:1 Langmuir model with mass transfer in Genedata Screener software. TREM-1 antibodies that met design goals and showed an affinity difference of less than 10-fold between human and cyno TREM-1 were identified, and their binding affinities are displayed in Table 5.

TABLE 5
TREM1 antibodies with <10-fold human and cyno TREM-1 affinity gap

| Antibody | Binding affinity for human TREM1 | | | Binding affinity for cyno TREM1 | | |
|---|---|---|---|---|---|---|
| ID | K_{D} (M) | kₐ (M⁻¹s⁻¹⁻) | k_{d} (s⁻¹) | K_{D} (M) | kₐ (M⁻¹s⁻¹⁻) | k_{d} (s⁻¹) |
| 3E12 | 5.70E−10 | 4.62E+05 | 2.63E−04 | 1.80E−09 | 4.89E+05 | 8.82E−04 |
| 34D1 | 8.81E−10 | 2.33E+05 | 2.05E−04 | 4.97E−09 | 2.58E+05 | 1.28E−03 |
| 30H2 | 8.98E−10 | 3.26E+05 | 2.93E−04 | 5.70E−09 | 2.14E+05 | 1.22E−03 |
| 44A5 | 4.04E−10 | 4.51E+05 | 1.82E−04 | 2.02E−09 | 7.51E+05 | 1.52E−03 |
| 46H7 | 4.57E−10 | 7.37E+05 | 3.36E−04 | 9.93E−10 | 1.14E+06 | 1.13E−03 |
| 49A2 | 7.67E−10 | 3.73E+05 | 2.86E−04 | 1.87E−09 | 7.73E+05 | 1.44E−03 |
| 50A12 | 4.85E−09 | 5.19E+05 | 2.52E−03 | 6.42E−09 | 6.87E+05 | 4.41E−03 |
| 57C10 | 8.24E−10 | 3.04E+05 | 2.51E−04 | 1.28E−09 | 1.87E+05 | 2.40E−04 |
| 57F5 | <2.81E−10* | 6.27E+05 | <1.76E−4* | <2.9E−10* | 6.07E+05 | <1.76E−4* |
| 61B12 | <5.57E−10* | 3.16E+05 | <1.76E−4* | 1.27E−09 | 3.91E+05 | 4.95E−04 |
| 61G5 | <2.85E−10* | 6.18E+05 | <1.76E−4* | 1.31E−09 | 3.64E+05 | 4.76E−04 |
| 63F8 | <2.20E−10* | 7.99E+05 | <1.76E−4* | 8.56E−10 | 7.52E+05 | 6.44E−04 |
| 64D7 | <2.50E−10* | 7.05E+05 | <1.76E−4* | 6.80E−10 | 5.19E+05 | 3.53E−04 |
| 66B8 | 7.98E−09 | 1.71E+05 | 1.36E−03 | 1.64E−09 | 1.54E+05 | 2.53E−04 |

*A < symbol designates that less than 10% of the TREM1 dissociated during the allotted 10-minute dissociation time, indicating that the k_{d} is <1.76E−4 s⁻¹. The < K_{D} (M) is calculated based on k_{d} <1.76E−4 s⁻¹.

Antibodies were selected based on binding to human or cyno TREM-1, lack of binding to TREM2, and their ability to block PGLYRP1 binding to TREM-1. 14 antibodies were selected for further study as set out in Table 6.

TABLE 6
Anti-TREM-1 antibody clones

| ID | Vh | Vlight | HCDR3 | Human KD | Cyno KD |
|---|---|---|---|---|---|
| 66B8 | VH1\|1-02/D6\|6- | VK1\|L5/JK5 | AGYSTSWKDWFDP | 7.98E−09 | 1.64E−09 |
| | 6\|RF1/JH5 | | | | |
| 44A5 | VH3\|3-23/D6\|6- | VK3\|L2/JK4 | AYYSNYLFDY | 4.04E−10 | 2.02E−09 |
| 46H7 | 6\|RF1/JH4 | VK3\|L2/JK1 | EFSSNSLFDY | 4.57E−10 | 9.93E−10 |
| 57F5 | VH3\|3-23/D1\|1- | VK1\|O12/JK4 | EGGSNRYFHH | <2.81E−10 | <2.9E−10 |
| 30H2 | 26\|RF3/JH1 | VK3\|L2/JK1 | VAGSNFFFDH | 8.98E−10 | 5.70E−09 |
| 3E12 | VH1\|1-02/D1\|1- | VL3\|3r/JL2 | DQRKTTVTPFEY | 5.70E−10 | 1.80E−09 |
| | 1\|RF1/JH4 | | | | |
| 61B12 | VH1\|1-08/D6\|6- | VK1\|O18/JK4 | GGISSSWHWAFDI | <5.57E−10 | 1.27E−09 |
| | 6\|RF1/JH3 | | | | |
| 63F8 | VH1\|1-08/D6\|6- | VK1\|O18/JK4 | GGRTSIWSFVFDY | <2.20E−10 | 8.56E−10 |
| | 13\|RF1/JH4 | | | | |
| 57C10 | VH1\|1-08/D6\|6- | VK1\|O18/JK4 | GGYTSAWRWYFDL | 8.24E−10 | 1.28E−09 |
| | 13\|RF1/JH2 | | | | |
| 34D1 | VH1\|1-08/D3\|3- | VK1\|O18/JK5 | GGYTSSWRWYFDL | 8.81E−10 | 4.97E−09 |
| | 22\|RF2/JH2 | | | | |
| 49A2 | VH3\|3-07/D1\|1- | VK3\|L2/JK4 | GGWRFES | 7.67E−10 | 1.87E−09 |
| | 1\|RF1/JH1 | | | | |
| 61G5 | VH1\|1-08/D7\|7- | VK1\|O18/JK5 | LIGYSSAWKWSFQH | <2.85E−10 | 1.31E−09 |
| | 27\|RF1/JH1 | | | | |
| 64D7 | VH1\|1-08/D6\|6- | VK1\|O18/JK4 | RRAVYRSSWEWYFDL | <2.50E−10 | 6.80E−10 |
| | 19\|RF2/JH2 | | | | |
| 50A12 | VH3\|3-07/D4\|4- | VK1\|L5/JK1 | DYGDSFDY | 4.85E−09 | 6.42E−09 |
| | 17\|RF2/JH4 | | | | |

TABLE 7
Anti TREM-1 Antibody Full Sequences

| | LIGHT | HEAVY | LIGHT | HEAVY |
|---|---|---|---|---|
| | CHAIN | CHAIN | CHAIN | CHAIN |
| | NT SEQ | NT SEQ | AA SEQ | AA SEQ |
| CLONE | ID NO: | ID NO: | ID NO: | ID NO: |
| 66B8 | 3 | 4 | 5 | 6 |
| 64D7 | 23 | 24 | 25 | 26 |
| 63F8 | 43 | 44 | 45 | 46 |
| 61G5 | 63 | 64 | 65 | 66 |
| 61B12 | 83 | 84 | 85 | 86 |
| 57F5 | 103 | 104 | 105 | 106 |
| 57C10 | 123 | 124 | 125 | 126 |
| 50A12 | 143 | 144 | 145 | 146 |
| 49A2 | 163 | 164 | 165 | 166 |
| 46H7 | 183 | 184 | 185 | 186 |
| 44A5 | 203 | 204 | 205 | 206 |
| 30H2 | 223 | 224 | 225 | 226 |
| 34D1 | 243 | 244 | 245 | 246 |
| 3E12 | 263 | 264 | 265 | 266 |
| 1B12 | 537 | 538 | 539 | 540 |

TABLE 8
TREM-1 Antibody Variable Region Sequences

| | VL DNA | VH DNA | VL AA | VH AA |
|---|---|---|---|---|
| CLONE | SEQ ID NO: | SEQ ID NO: | SEQ ID NO: | SEQ ID NO: |
| 66B8 | 19 | 20 | 21 | 22 |
| 64D7 | 39 | 40 | 41 | 42 |
| 63F8 | 59 | 60 | 61 | 62 |
| 61G5 | 79 | 80 | 81 | 82 |
| 61B12 | 99 | 100 | 101 | 102 |
| 57F5 | 119 | 120 | 121 | 122 |
| 57C10 | 139 | 140 | 141 | 142 |
| 50A12 | 159 | 160 | 161 | 162 |
| 49A2 | 179 | 180 | 181 | 182 |
| 46H7 | 199 | 200 | 201 | 202 |
| 44A5 | 219 | 220 | 221 | 222 |
| 30H2 | 239 | 240 | 241 | 242 |
| 34D1 | 259 | 260 | 261 | 262 |
| 3E12 | 279 | 280 | 281 | 282 |

TABLE 9
TREM-1 Antibody Clone Light Chain CDR Sequences

| | LCDR1 NT | LCDR2 NT | LCDR3 NT | LCDR1 AA | LCDR 2 AA | LCDR3 AA |
|---|---|---|---|---|---|---|
| CLONE | SEQ ID NO: | SEQ ID NO: | SEQ ID NO: | SEQ ID NO: | SEQ ID NO: | SEQ ID NO: |
| 66B8 | 7 | 8 | 9 | 10 | 11 | 12 |
| 64D7 | 27 | 28 | 29 | 30 | 31 | 32 |
| 63F8 | 47 | 48 | 49 | 50 | 51 | 52 |
| 61G5 | 67 | 68 | 69 | 70 | 71 | 72 |
| 61B12 | 87 | 88 | 89 | 90 | 91 | 92 |
| 57F5 | 107 | 108 | 109 | 110 | 111 | 112 |
| 57C10 | 127 | 128 | 129 | 130 | 131 | 132 |
| 50A12 | 147 | 148 | 149 | 150 | 151 | 152 |
| 49A2 | 167 | 168 | 169 | 170 | 171 | 172 |
| 46H7 | 187 | 188 | 189 | 190 | 191 | 192 |
| 44A5 | 207 | 208 | 209 | 210 | 211 | 212 |
| 30H2 | 227 | 228 | 229 | 230 | 231 | 232 |
| 34D1 | 247 | 248 | 249 | 250 | 251 | 252 |
| 3E12 | 267 | 268 | 269 | 270 | 271 | 272 |
| 1B12 | 541 | 542 | 543 | 544 | 545 | 546 |

TABLE 10
TREM-1 Antibody Clone Heavy Chain CDR Sequences

| | HCDR1 NT | HCDR2 NT | HCDR3 NT | HCDR1 AA | HCDR 2 AA | HCDR3 AA |
|---|---|---|---|---|---|---|
| CLONE | SEQ ID NO: | SEQ ID NO: | SEQ ID NO: | SEQ ID NO: | SEQ ID NO: | SEQ ID NO: |
| 66B8 | 13 | 14 | 15 | 16 | 17 | 18 |
| 64D7 | 33 | 34 | 35 | 36 | 37 | 38 |
| 63F8 | 53 | 54 | 55 | 56 | 57 | 58 |
| 61G5 | 73 | 74 | 75 | 76 | 77 | 78 |
| 61B12 | 93 | 94 | 95 | 96 | 97 | 98 |
| 57F5 | 113 | 114 | 115 | 116 | 117 | 118 |
| 57C10 | 133 | 134 | 135 | 136 | 137 | 138 |
| 50A12 | 153 | 154 | 155 | 156 | 157 | 158 |
| 49A2 | 173 | 174 | 175 | 176 | 177 | 178 |
| 46H7 | 193 | 194 | 195 | 196 | 197 | 198 |
| 44A5 | 213 | 214 | 215 | 216 | 217 | 218 |
| 30H2 | 233 | 234 | 235 | 236 | 237 | 238 |
| 34D1 | 253 | 254 | 255 | 256 | 257 | 258 |
| 3E12 | 273 | 274 | 275 | 276 | 277 | 278 |
| 1B12 | 547 | 548 | 549 | 550 | 551 | 552 |

### Example 2-Reformatting of Anti-TREM-1 Antibodies

Lead anti-TREM-1 antibodies from the XenoMouse® campaign were converted to an antibody format of the IgG1z subtype by fusing the VL domain of kappa light chains to CK domain, the VL domain of lambda light chains to CL domain, and VH domains to the CH1-CH2-CH3(221-447) sequence. The CH2 domain of this antibody isotype has been engineered for reduced effector function by incorporating an N297G mutation and for improved thermostability through an engineered disulfide bond (1R292C, V302C); this antibody isotype is designated IgG1z SEFL2. The lead anti-TREM-1 antibodies were additionally engineered to remove “hotspots,” or residues that were computationally predicted or empirically determined to negatively impact the molecule's expression, purification, thermal stability, colloidal stability, long-term storage stability, in vivo pharmacokinetics, and/or immunogenicity. A variety of amino acid mutations at these hotspots were designed based on conservation, co-variation, chemical similarity, predictions from structural modeling, and prior knowledge from other antibody engineering campaigns. A small panel of rationally designed engineered antibodies were designed that included both single mutations and combinations of mutations.

Recombinant expression constructs for the rationally designed panel of hotspot engineered variants were produced using Golden Gate cloning to assemble 1) synthetic DNA fragments comprising the antibody variable domains, 2) previously cloned “parts vectors” containing the necessary constant domains (i.e., CK or CL, CH1-CH2-CH3(118-447) (R292C, N297G, V302C)), and 3) a mammalian expression vector backbone. Heavy chains (HCs) were assembled into a vector backbone with a puromycin selection cassette and light chains (LCs) were assembled into a vector backbone with a hygromycin selection cassette. The HC and LC expression vectors were co-transfected in a 1:1 ratio in CHO-K1 cells using Lipofectamine LTX (Gibco), and stable pools were generated by passaging every 2-3 days in the presence of 10 ug/mL puromycin and 500 ug/mL hygromycin until cell viability was >90% (Vi-CELL BLU, Beckman Coulter). Stable pools were seeded in production media at 2e6 viable cells per mL of culture and incubated at 36° C. in 5% CO₂ for 6 days. Cell supernatant was harvested by centrifugation and antibodies were purified by magnetic bead affinity chromatography using AmMag™ Protein A Magnetic Beads (GenScript) or MAG SEPHAROSE™ PrismA (Cytiva). The identity of each variant was confirmed by intact mass spectrometry. For each variant, the expression titer in conditioned medium was measured by ForteBio OCTET® (Pall Life Sciences) using Protein A sensors. The percent of high molecular weight (% HMW) material present after Protein A affinity chromatography was measured by analytical size exclusion chromatography, and the % target protein purity was measured by non-reduced microcapillary electrophoresis (MCE NR) using a LabChip® GXII (Perkin Elmer). Data for the variants prepared by this process appear in Table 11.

TABLE 11
Panel of rationally designed hotspot engineered TREM1 antibodies

| | Heavy chain | Light chain | % HMW | % target |
|---|---|---|---|---|
| Antibody ID | SEQ ID NO. | SEQ ID NO: | material | purity |
| TREM1_30H2.001_huIgGz SEFL2 | 283 | 715 | 1 | 100 |
| TREM1_34D1.001_huIgGz SEFL2 | 285 | 716 | 3 | 99 |
| TREM1_3E12.001_huIgGz SEFL2 | 287 | 2545717 | 4 | 95 |
| TREM1_44A5.001_huIgGz SEFL2 | 289 | 2546718 | 1 | 100 |
| TREM1_46H7_huIgGz SEFL2 | 291 | 2547719 | 4 | 98 |
| TREM1_49A2.001_huIgGz SEFL2 | 293 | 2548720 | 3 | 97 |
| TREM1_50A12_huIgGz SEFL2 | 295 | 2549721 | 2 | 99 |
| TREM1_57C10.001_huIgGz SEFL2 | 297 | 2550722 | 1 | 99 |
| TREM1_57F5.001_huIgGz SEFL2 | 299 | 2551723 | 1 | 100 |
| TREM1_61B12.001_huIgGz SEFL2 | 301 | 2552724 | 2 | 99 |
| TREM1_61G5.001_huIgGz SEFL2 | 303 | 2553725 | 1 | 100 |
| TREM1_63F8.001_huIgGz SEFL2 | 305 | 2554726 | 1 | 99 |
| TREM1_64D7.001_huIgGz SEFL2 | 307 | 2555727 | 0 | 99 |
| TREM1_66B8_huIgGz SEFL2 | 309 | 2556728 | 1 | 99 |
| TREM1_34D1.001_huIgGz SEFL2 | 311 | 2557729 | 3 | 99 |
| TREM1_44A5.001_huIgGz SEFL2 | 313 | 2558730 | 3 | 98 |
| TREM1_50A12_huIgGz SEFL2 | 315 | 2559731 | 0 | 98 |
| TREM1_57C10.001_huIgGz SEFL2 | 317 | 2560732 | ND | ND |
| TREM1_61G5.001_huIgGz SEFL2 | 319 | 2561733 | 9 | 97 |
| TREM1_64D7.001_huIgGz SEFL2 | 321 | 2562734 | 0 | 100 |
| TREM1_66B8_huIgGz SEFL2 | 323 | 2563735 | ND | ND |
| TREM1_57C10(VH: N83D_N87S, VL: S77R_C83S_F105Y_E148K) | 325 | 256473 | ND | ND |
| TREM1_57C10(VH: N83D, VL: S77R_C83S_F105Y_E148K) | 327 | 737 | ND | ND |
| TREM1_66B8(VH: S81T_W115Y, VL: F7S) | 329 | 738 | 2 | 99 |
| TREM1_66B8(VH: S81T_W115H, VL: F7S) | 331 | 739 | 0 | 93 |
| TREM1_66B8(VH: S81T_W115F, VL: F7S) | 333 | 740 | 1 | 97 |
| TREM1_66B8(VH: S81T, VL: F7S_Q48P) | 335 | 741 | ND | ND |
| TREM1_66B8(VH: S81T, VL: F7S) | 337 | 742 | ND | ND |
| TREM1_19337(VH: N83D, VL: E15V_Y89F) | 339 | 743 | 1 | 100 |
| TREM1_19337(VH: N83D, VL: E15V_E46Q) | 341 | 744 | 1 | 98 |
| TREM1_19337(VH: N83D, VL: E15V) | 343 | 745 | 1 | 99 |
| TREM1_61G5(VH: N83D_W116Y, VL: Q55L_I101F) | 345 | 746 | 1 | 97 |
| TREM1_61G5(VH: N83D_W116Y, VL: Q55L) | 347 | 747 | 2 | 98 |
| TREM1_61G5(VH: N83D_W116F, VL: Q55L_I101F) | 349 | 748 | 2 | 97 |
| TREM1_61G5(VH: N83D_W116F, VL: Q55L) | 351 | 749 | ND | ND |
| TREM1_61G5(VH: N83D_I86T_W116Y, VL: Q55L_I101F) | 353 | 750 | 1 | 98 |
| TREM1_61G5(VH: N83D_I86T, VL: Q55L_I101F) | 355 | 751 | 1 | 100 |
| TREM1_61G5(VH: N83D, VL: Q55L_I101F) | 357 | 752 | 2 | 99 |
| TREM1_61G5(VH: N83D, VL: Q55L) | 359 | 753 | 2 | 99 |
| TREM1_50A12(VH: F90Y_S135V), | 361 | 754 | 0 | 98 |
| TREM1_50A12(VH: F90Y_S135A), | 363 | 755 | 0 | 84 |
| TREM1_50A12(VH: F90Y_D112E), | 365 | 756 | 0 | 99 |
| TREM1_24B1(VH: T48P_N83D, VL: N20T_N53K_A88D_I101F) | 367 | 757 | 0 | 97 |
| TREM1_24B1(VH: S68A_N83D, VL: N20T) | 369 | 758 | 0 | 99 |
| TREM1_24B1(VH: S65A_S68A_N83D, VL: N20T_N53K_A88D_I101F) | 371 | 759 | 1 | 97 |
| TREM1_24B1(VH: S65A_N83D, VL: N20T) | 373 | 760 | 0 | 100 |
| TREM1_24B1(VH: N83D_W134Y, VL: N20T_N53K_A88D_I101F) | 375 | 761 | 0 | 99 |
| TREM1_24B1(VH: N83D_W134H, VL: N20T_N53K_A88D_I101F) | 377 | 762 | 1 | 98 |
| TREM1_24B1(VH: N83D_W134F, VL: N20T_N53K_A88D_I101F) | 379 | 763 | 0 | 97 |
| TREM1_24B1(VH: N83D, VL: N20T_N53K_A88D_F91L_I101F) | 381 | 764 | 0 | 97 |
| TREM1_24B1(VH: N67Q_N83D, VL: N20T) | 383 | 765 | 0 | 93 |
| TREM1_24B1(VH: N61Q_N83D, VL: N20T) | 385 | 766 | 0 | 98 |
| TREM1_24B1(VH: N61Q_N67Q_N83D, VL: N20T_N53K_A88D_I101F) | 387 | 767 | 1 | 98 |
| TREM1_3E12(VH: W77R_T97R, VL: N40Q_C42S_L46Q_I53V_I90T) | 389 | 768 | ND | ND |
| TREM1_3E12(VH: W77R_T97R, VL: N40Q_C42S_I53V_I90T) | 391 | 769 | ND | ND |
| TREM1_3E12(VH: W77R_T97R, VL: C42G_L46Q_I53V_I90T) | 393 | 770 | ND | ND |
| TREM1_3E12(VH: W77R_T97R, VL: C42A_L46Q_I53V_I90T) | 395 | 771 | ND | ND |
| TREM1_3E12(VH: W77R, VL: N40Q_C42S_I53V_I90T) | 397 | 772 | ND | ND |
| TREM1_44A5(VLI10T_V74I_G81S_S98P_C109F_D146E_L147I) | 399 | 773 | 4 | 99 |
| TREM1_44A5(VLI10T_V74I_G81S_C108F_W111Y_D146E_L147I) | 401 | 774 | 3 | 99 |
| TREM1_44A5(VLI10T_V74I_G81S_C108F_W111F_D146E_L147I) | 403 | 775 | 2 | 99 |
| TREM1_44A5(VLI10T_V74I_G81S_C108F_W111H_D146E_L147I) | 405 | 776 | 3 | 99 |
| TREM1_44A5(VLI10T_V74I_G81S_S98P_C108F_W111Y_D146E_L147I) | 407 | 777 | 5 | 98 |

A subset of three anti-TREM-1 lead antibodies from the XENOMOUSE® campaign (30H2 (19330), 49A2 (19333), and 46H7 (19332)) were also engineered through yeast display for improved manufacturability with retained binding to TREM-1. For each antibody, libraries were generated in which every possible adjacent pair of residues in all six CDRs were simultaneously mutated to all possible amino acids through use of degenerate NNK codons. The libraries were displayed on the surface of yeast derivative of BJ5464, wherein the Fd domain was fused to the N-terminus of alpha-agglutinin and the LC was not fused to the yeast surface. Efficiency of display was measured by binding of ALEXA FLUOR® 647 conjugated anti-Fab antibody. Libraries were sorted using fluorescence activated cell sorting (FACS) for high binding to biotin conjugated recombinant TREM-1 ECD) using streptavidin PE as fluorescence secondary. The variable domains present in the sorted binding/display double positive pools and display positive pools were amplified with primers specific to the framework 1 (FW1) and FW4 domains of the HC and LC and submitted to NGS analysis on an Illumina MiSeq for a 2×300 bp run. Mutations were selected after processing the data through a common frequency analysis where the ratio of positive binding amino acid frequencies are divided by positive display amino acid frequencies which is then normalized to the parental sequence ratio. The sequences for which the enrichment values were greater than or equal to the parental sequence were considered beneficial or tolerated diversity and were used for additional rational antibody engineering post affinity maturation.

Top display engineered variable domains were converted to the IgG1z SEFL2 isotype and cloned using Golden Gate cloning to assemble 1) synthetic DNA fragments comprising the antibody variable domains, 2) previously cloned “parts vectors” containing the necessary constant domains (i.e., CK or CL, CH1-CH2-CH3(118-447) (R292C, N297G, V302C)), and 3) a mammalian expression vector backbone. Heavy chains (HCs) were assembled into a vector backbone with a puromycin selection cassette and light chains (LCs) were assembled into a vector backbone with a hygromycin selection cassette. The HG and LC expression vectors were co-transfected in a 1:1 ratio in CHO-K1 cells using Lipofectamine LTX (Gibco) and stable pools were generated by passaging every 2-3 days in the presence of 10 ug/mL puromycin and 500 ug/mL hygromycin until cell viability was >90% (Vi-CELL BLU, Beckman Coulter). Stable pools were seeded in production media at 2e6 viable cells per mL of culture and incubated at 36° C. in 5% CO₂ for 6 days. Antibodies were purified by magnetic bead affinity chromatography using AMMAG™ Protein A Magnetic Beads (GenScript). The identity of each molecule was confirmed by intact mass spectrometry. The percent of high molecular weight (% HMW) material present after Protein A affinity chromatography was measured by analytical size exclusion chromatography, and the % target protein purity was measured by non-reduced microcapillary electrophoresis (MOE NR) using a LabChip® GXII (Perkin Elmer). Data for the variants prepared by yeast display hot-spot engineering appear in Table 12.

TABLE 12
Yeast display hot-spot engineered variant TREM1 antibodies

| | HC SEQ | LC SEQ | % | % target |
|---|---|---|---|---|
| Antibody ID | ID NO. | ID NO. | HMW | purity |
| TREM1_30H2(VH: E17G_T24A, VL: S39A_L141Q_Q144K) | 411 | 587 | 21 | 99 |
| TREM1_30H2(VH: E17G_T24A, VL: S39I_L141Q_Q144K) | 413 | 589 | 22 | 85 |
| TREM1_30H2(VH: E17G_T24A, VL: S39H_L141Q_Q144K) | 415 | 591 | 21 | 99 |
| TREM1_30H2(VH: E17G_T24A, VL: S39V_L141Q_Q144K) | 417 | 593 | 20 | 87 |
| TREM1_30H2(VH: E17G_T24A, VL: M111I_L141Q_Q144K) | 419 | 595 | 38 | 99 |
| TREM1_30H2(VH: E17G_T24A, VL: M111L_L141Q_Q144K) | 421 | 597 | 29 | 99 |
| TREM1_30H2(VH: E17G_T24A, VL: W111F_L141Q_Q144K) | 423 | 599 | 15 | 99 |
| TREM1_30H2(VH: E17G_T24A, VL: W137Y_L141Q_Q144K) | 425 | 601 | 17 | 99 |
| TREM1_30H2(VH: E17G_T24A, VL: W137H_L141Q_Q144K) | 427 | 603 | 17 | 99 |
| TREM1_30H2(VH: E17G_T24A, VL: W137N_L141Q_Q144K) | 429 | 605 | 17 | 99 |
| TREM1_30H2(VH: V4L_E17G_T24A_A99E, VL: L141Q_Q144K) | 431 | 607 | 3 | 99 |
| TREM1_30H2(VH: V4L_E17G_T24A_A99E, VL: S39A_M111L_L141Q_Q144K) | 433 | 609 | 15 | 95 |
| TREM1_30H2(VH: V4L_E17G_T24A_A99E, VL: S39A_M111L_W137H_L141Q_Q144K) | 435 | 611 | 3 | 98 |
| TREM1_30H2(VH: V4L_E17G_T24A_A99E, VL: S39A_M111L_W137N_L141Q_Q144K) | 437 | 613 | 3 | 97 |
| TREM1_30H2(VH: V4L_E17G_T24A_A99E, VL: S39A_M110L_W111F_L141Q_Q144K) | 439 | 615 | 3 | 97 |
| TREM1_30H2(VH: V4L_E17G_T24A_A99E, VL: S39A_M110L_W111F_W137N_L141Q_Q144K) | 441 | 617 | 2 | 96 |
| TREM1_30H2(VH: V4L_E17G_T24A_A99E, VL: S39A_M110L_W111F_W137H_L141Q_Q144K) | 443 | 619 | 38 | 98 |
| TREM1_30H2(VH: V4L_E17G_T24A_A99E, VL: S39H_M111L_L141Q_Q144K) | 445 | 621 | 12 | 99 |
| TREM1_30H2(VH: V4L_E17G_T24A_A99E, VL: S39H_M111L_W137N_L141Q_Q144K) | 447 | 623 | 3 | 98 |
| TREM1_30H2(VH: V4L_E17G_T24A_A99E, VL: S39H_M111L_W137H_L141Q_Q144K) | 449 | 625 | 2 | 98 |
| TREM1_30H2(VH: V4L_E17G_T24A_A99E, VL: S39H_M110L_W111F_L141Q_Q144K) | 451 | 627 | 3 | 98 |
| TREM1_30H2(VH: V4L_E17G_T24A_A99E, VL: $39H_M110L_W111F_W137N_L141Q_Q144K) | 453 | 629 | 3 | 98 |
| TREM1_30H2(VH: V4L_E17G_T24A_A99E, VL: S39H_M110L_W111F_W137H_L141Q_Q144K) | 454 | 631 | 4 | 98 |
| TREM1_46H7(VL: R50Q) | 457 | 633 | 4 | 99 |
| TREM1_46H7(VL: S39A_R50Q) | 459 | 635 | 4 | 99 |
| TREM1_46H7(VL: S391_R50Q) | 461 | 637 | 4 | 98 |
| TREM1_46H7(VL: S39H_R50Q) | 463 | 639 | 3 | 99 |
| TREM1_46H7(VL: S39V_R50Q) | 465 | 641 | 4 | 99 |
| TREM1_46H7(VL: R50Q_W111F) | 467 | 643 | 4 | 99 |
| TREM1_46H7(VL: R50Q_W137Y) | 469 | 645 | 4 | 99 |
| TREM1_46H7(VL: R50Q_W137H) | 471 | 647 | 5 | 99 |
| TREM1_46H7(VL: R50Q_W137N) | 473 | 649 | 9 | 99 |
| TREM1_46H7(VH: M12L, VL: R50Q) | 475 | 651 | 4 | 99 |
| TREM1_46H7(VH: M12L, VL: S39A_R50Q) | 477 | 653 | 3 | 99 |
| TREM1_46H7(VH: M12L, VL: S39A_R50Q_W111F) | 479 | 655 | 4 | 99 |
| TREM1_46H7(VH: M12L, VL: S39A_R50Q_W137Y) | 481 | 657 | 0 | 0 |
| TREM1_46H7(VH: M12L, VL: S39A_R50Q_W137H) | 483 | 659 | 4 | 99 |
| TREM1_46H7(VH: M12L, VL: S39A_R50Q_W111F_W137Y) | 485 | 661 | 4 | 99 |
| TREM1_46H7(VH: M12L, VL: S39A_R50Q_W111F_W137H) | 487 | 663 | 4 | 99 |
| TREM1_46H7(VH: M12L, VL: S39H_R50Q) | 489 | 665 | 3 | 99 |
| TREM1_46H7(VH: M12L, VL: S39H_R50Q_W111F) | 491 | 667 | 3 | 99 |
| TREM1_46H7(VH: M12L, VL: S39H_R50Q_W137N) | 493 | 669 | 9 | 99 |
| TREM1_46H7(VH: M12L, VL: S39H_R50Q_W137Y) | 495 | 671 | 3 | 99 |
| TREM1_46H7(VH: M12L, VL: S39H_R50Q_W111F_W137N) | 497 | 673 | 6 | 99 |
| TREM1_46H7(VH: M12L, VL: S39H_R50Q_W111F_W137Y) | 499 | 675 | 3 | 99 |
| TREM1_49A2(VH: M55V_A143T, VL: V78F_L139F) | 501 | 677 | 16 | 99 |
| TREM1_49A2(VH: M55V_A143T, VL: S48P_V78F_L139F) | 503 | 679 | 16 | 99 |
| TREM1_49A2(VH: M55V_A143T, VL: V78F_F91L_L139F) | 505 | 681 | 14 | 99 |
| TREM1_49A2(VH: M55V_M58I_A143T, VL: V78F_L139F) | 507 | 683 | 21 | 98 |
| TREM1_49A2(VH: M55V_A143T, VL: V78F_W135F_L139F) | 509 | 685 | 21 | 99 |
| TREM1_49A2(VH: M55V_D61H_A143T, VL: V78F_L139F) | 511 | 687 | 4 | 99 |
| TREM1_49A2(VH: M55V_G65A_A143T, VL: V78F_L139F) | 513 | 689 | 10 | 99 |
| TREM1_49A2(VH: M55V_G65L_A143T, VL: V78F_L139F) | 515 | 691 | 6 | 99 |
| TREM1_49A2(VH: M55V_D72E_A143T, VL: V78F_L139F) | 517 | 693 | 15 | 99 |
| TREM1_49A2(VH: M55V_M58I_G65A_S73A_A143T, VL: V78F_F91L_W135F_L139F) | 519 | 695 | 16 | 99 |
| TREM1_49A2(VH: M55V_S73A_A143T, VL: V78F_L139F) | 521 | 697 | 22 | 98 |
| TREM1_49A2(VH: M55V_M58I_G65A_S73A_A143T, VL: S48P_V78F_F91L_L139F) | 523 | 699 | 16 | 99 |
| TREM1_49A2(VH: M55V_M58I_G65A_S73A_A143T, VL: V78F_F91L_L139F) | 525 | 701 | 22 | 99 |
| TREM1_49A2(VH: M55V_M58I_G65A_D72E_A143T, VL: S48P_V78F_F91L_L139F) | 527 | 703 | 15 | 99 |
| TREM1_49A2(VH: M55V_M58I_G65A_D72E_A143T, VL: V78F_F91L_L139F) | 529 | 705 | 16 | 99 |
| TREM1_49A2(VH: M55V_M58I_G65A_D72E_A143T, VL: S48P_V78F_F91L_W135F_L139F) | 531 | 707 | 21 | 99 |
| TREM1_49A2(VH: M55V_M58I_G65A_D72E_A143T, VL: V78F_F91L_W135F_L139F) | 533 | 709 | 21 | 98 |
| TREM1_49A2(VL: L139F) | 535 | 711 | 12 | 98 |

Comparison of bivalent and monovalent anti-TREM-1 mAbs in HEK293 cells expressing TREM-1/DAP12 was carried out by analysis of the phosphorylation levels of Syk kinase in cells using a p-Syk ALPHALISA® (Perkin-Elmer). Briefly, HEK293 cells stably expressing human TREM1 and DAP12 were cultured in DMEM/F12 Ham medium (Corning) supplemented with 10% dialyzed FBS (Gibco), 2 mM GlutaMAX (Gibco), 2 mM L-glutamine (Sigma), 1% penicillin/streptomycin (Gibco) and 0.1 mg/mL zeocin (Gibco) at 37° C./5% CO₂. One day prior to experiment, the cells were detached using Trypsin-EDTA and centrifuged at 400×g for 5 minutes. The cell pellet was resuspended in complete medium before a second centrifugation at 400×g for 5 minutes. After centrifugation the cells were resuspended in complete medium at a concentration of 1×10⁶ cells/mL and seeded to CELLBIND® 96-well clear flat bottom polystyrene plates (Corning) at a final volume of 100 μL/well or 50,000 cells/well. The seeded plates were incubated at 37° C./5% CO₂ overnight for 18 to 24 hours. On the day of experiment, 70 μL of medium was removed from each well. TREM1 antibodies were diluted to 3 times the highest final concentration in a 4-fold serial dilution in assay medium (DMEM/F12 HAM medium supplemented with 10% heat inactivated FBS). The crosslinking reagent Protein G (Sigma) was prepared at 3 times the final concentration using assay medium. The titrated TREM1 antibodies were mixed 1 to 1 with either Protein G or assay medium and 60 μL of each TREM1 antibodies+/− crosslinking reagent was added to each well containing cells. These plates were incubated at room temperature for 1 hour before all medium was removed from the wells and the cells were then lysed using 25 μL/well of lysis buffer (M-Per Mammalian Protein Extraction Reagent and 1× Halt Protease/Phosphatases Inhibitor). The cells were incubated with lysis buffer on ice for 1 hour before 5 μL of cell lysate was transferred to each well of a 384-well white plate (PerkinElmer) containing AlphaLISA® acceptor cocktail (1 nM anti-pSyk, rabbit IgG (anti-phosphoSyk(Tyr525/526) (Clone C87C1)) (Cell Signaling Technology), 1 nM biotin-anti-Syk, mouse IgG (Clone 4D10) (BD Biosciences), 10 μg/mL anti-rabbit-IgG AlphaLISA® acceptor beads (PerkinElmer), and 1× Halt Inhibitor, in 1× AlphaLISA8 Immunoassay Buffer (PerkinElmer)). The plates were further incubated on ice for 2 hours before 5 μL of AlphaLISA® donor cocktail (streptavidin-alpha-donor beads (PerkinElmer) in 1× Immunoassay Buffer) was added at 40 μg/mL final concentration and incubated at room temperature in the dark for 1 hour. After incubation, phospho-Syk (pSyk) signal was detected via FRET (fluorescence resonance energy transfer) using ENVISION® plate reader (PerkinElmer). Results were calculated by ratio of sample pSyk signal/basal pSyk signal with potency and max signal being compared between the variations of each antibody. Bivalent anti-TREM-1 mAbs treatment alone induces weak signals in TREM-1/DAP12-expressing cells. No pSyk signal was observed with monovalent anti-TREM-1 mAbs, and cross-linking bivalent and monovalent anti-TREM-1 mAbs with protein G result in pSyk induction.

Clading and Alignment of Antibody Sequences: The VH and VL domains of input antibody sequences were extracted and aligned to a structure-based IgG numbering system based on Honegger and Pluckthun (J Mol Biol. 309(3):657-70, 2001). A distance matrix was generated from the composite multiple sequence alignment using an uncorrected model, in which the distance between two sequences is the fraction of mismatches in both the VH and VL domains. Finally, the distance matrix was used to construct a tree via the UPGMA (unweighted pair group method with arithmetic mean) method (Sokal and Michener, University of Kansas Science Bulletin. 38: 1409-1438, 1958), and related sequences were grouped based on a branch traversal limit of 0.2. Alignments were refined manually based on chemical similarity of amino acids to generate consensus CDR sequences for each cladded group resulting in the sequences shown in Table 13.

TABLE 13
Consensus Sequences of TREM-1 Antigen Binding Proteins

| Alignment | | | | | | |
|---|---|---|---|---|---|---|
| Group | LCDR1 | LCDR2 | LCDR3 | HCDR1 | HCDR2 | HCDR3 |
| Group I | X₁ASQSX₂X₃ | GAX₁X₂RAT | QX₁X₂X₃X₄X₅ | X₁X₂X₃MX₄ | X₁X₂X₃X₄X₅X₆ | X₁X₂X₃X₄X₅X₆ |
| | X₄NLA | (SEQ ID | X₆PX₇T | (SEQ ID | X₇X₈X₉YYX₁₀ | X₇X₈X₉YYX₁₀ |
| | (SEQ ID | NO: 554), | (SEQ ID | NO: 556) | X₁₁X₁₂VKG | (SEQ ID |
| | NO: 553) | | NO: 555) | | (SEQ ID | NO: 557) |
| | | | | | NO: 559) | |
| | RASQSVNS | GASTRAT | QQFKNWP | AYAMS | TSGSGSTT | VAGSNFLF |
| | NLA (SEQ | (SEQ ID | PT (SEQ ID | (SEQ ID | YYADSVKG | DY (SEQ ID |
| | ID NO: 566) | NO: 573); | NO: 576); | NO: 581) | (SEQ ID | NO: 842). |
| | QASQDIHL | | | | NO: 584) | |
| | N (SEQ ID | | | | | |
| | NO: 569) | | | | | |
| Group II | RASQGIRK | AASRLQS | LQAHGFP | SYWMS | NIKQDGSE | DYGDSFDY |
| | WLA (SEQ | (SEQ ID | WT (SEQ ID | (SEQ ID | EYYVDSVK | (SEQ ID |
| | ID NO: 570) | NO: 575) | NO: 2224); | NO: 583) | G (SEQ ID | NO: 845) |
| | | | QFWPPWT | | NO: 586); | |
| | | | (SEQ ID | | TSGSGTYY | |
| | | | NO: 580) | | ADSVKG | |
| | | | | | (SEQ ID | |
| | | | | | NO: 841) | |
| Group III | QASX₁DIX₂ | X₁X₂X₃X₄LE | QX₁YX₃X₄X₅ | X₁YDIN | X₁X₂NPX₃X₄ | X₁X₂X₃X₄X₅X₆ |
| | X₃X₄LN | T (SEQ ID | PX₆T (SEQ | (SEQ ID | GX₅X₆GX₇X₈ | X₇X₈X₉X₁₀ |
| | (SEQ ID | NO: 560) | ID NO: 561) | NO: 563) | X₉X₁₀FX₁₁X₁₂ | X₁₁X₁₂FX₁₃X₁₄ |
| | NO: 558) | | | | (SEQ ID | (SEQ ID |
| | | | | | NO: 564) | NO: 565) |
| | QASQDIRK | DASNLET | QHYDNLPI | RYDIN | WMNPNSG | GGYTSSW |
| | HLN (SEQ | (SEQ ID | T (SEQ ID | (SEQ ID | NSSVQKFR | RWYFDL |
| | ID | NO: 574); | NO: 577); | NO: 582) | G (SEQ ID | (SEQ ID |
| | NO: 567); | | QQYDNLPL | | NO: 585) | NO: 843); |
| | | | T (SEQ ID | | | GGYTSSW |
| | | | NO: 579 | | | SRWYFDL |
| | | | | | | (SEQ ID |
| | | | | | | NO: 844) |

### Example 3—Efficacy of TREM-1 Antibodies Blocking Ligand Mediated Signaling in Human Peripheral Blood Mononuclear Cells (PBMC)

TREM-1 antibodies were tested for the ability to block signaling by inhibition of ligand binding to the TREM-1 receptor in human PBMCs. Frozen human PBMCs (IQ Biosciences) were thawed, washed and resuspended in complete cell culture media (RPMI/10% FBS/2 mM GlutaMax/1 mM Sodium Pyruvate/44 uM p-mercaptoethanol/1×DNAse I). PBMCs were seeded in 96 well cell culture plates at 100K/well and allowed to equilibrate for at least 30 minutes at 37° C. Thirteen anti-TREM-1 antibodies were diluted (3× serial dilution) in RPMI/10% FBS to generate final antibody concentrations ranging from 0.000017 nM to 3 nM. Antibody 57F5 was excluded from this analysis because in prior iterations of this assay that utilized higher concentrations of antibody, it failed to demonstrate any inhibition of signaling. Antibodies were pre-incubated with PBMCs for 30 minutes before addition of the TREM-1 ligand. Peptidoglycan recognition protein 1 (PGLYRP1) complexed with peptidoglycan (PGN) is one of several described TREM-1 ligands whose engagement with the TREM-1 receptor triggers the production of inflammatory cytokines (e.g. TNFα) and was used in these assays. To complex the proteins, human PGLYRP1 (R&D Systems) was combined with soluble PGN derived from E. coli (InvivoGen) in a 1.25:2 ratio and incubated for 45 minutes at 37° C. Complexed PGLYRP1/PGN was added to PBMC/antibody and incubated overnight at 37° C. The following day, cell media was collected and assayed for TNFα by Human TNFα Alphalisa proximity assay (Perkin Elmer). Light emission at 615 nm was measured on an Envision 2103 multilabel plate reader. IC50 values were calculated using GraphPad Prism (v8.4.3) and are presented for two separate assays in Table 14 below.

TABLE 14
TREM-1 Antibody Inhibition of Ligand Mediated Signaling
in Human PBMCs as Measured by TNFα Release

| | Ab IC50-Human PBMC | Ab IC50-Human PBMC |
|---|---|---|
| Antibody ID | (Trial 1) pM | (Trial 2) pM |
| 57C10 | 8.8 | 7.2 |
| 30H2 | 6.9 | nd |
| 61B12 | 8.0 | nd |
| 61G5 | 10.2 | 2.7 |
| 63F8 | 3.8 | 3.1 |
| 64D7 | 4.3 | 2.4 |
| 34D1 | 10.0 | 6.9 |
| 44A5 | 3.5 | 6.1 |
| 46H7 | 6.3 | 4.4 |
| 49A2 | 3.1 | 3.3 |
| 66B8 | 31.6 | nd |
| 50A12 | 3.3 | 3.6 |
| 57F5 | nd | nd |
| 3E12 | no inhibition | nd |

These results show that the TREM-1 antibodies bind human TREM-1, and importantly demonstrate inhibition of ligand-induced TREM-1 activation in human primary cells (PBMCs).

### Example 4—Efficacy of TREM-1 Antibodies Blocking Ligand Mediated Signaling in Cynomolgus Monkey Peripheral Blood Mononuclear Cells (PBMC)

TREM-1 antibodies were assayed for blocking signaling by the inhibition of ligand binding to the TREM-1 receptor in cynomolgus monkey PBMCs. Frozen cynomolgus monkey PBMCs (IQ Biosciences) were thawed, washed and resuspended in complete cell culture media (RPMI/10% FBS/2 mM GlutaMax/1 mM Sodium Pyruvate/44 uM p-mercaptoethanol/1×DNAse I). PBMCs were seeded in 96 well cell culture plates at 100K/well and allowed to equilibrate for at least 30 minutes at 37° C. Fourteen anti-TREM-1 antibodies were diluted (3× serial dilution) in RPMI/10% FBS to generate final antibody concentrations ranging from 0.00017 nM to 30 nM. Antibodies were pre-incubated with PBMCs for 30 minutes before addition of the TREM-1 ligand. Peptidoglycan recognition protein 1 (PGLYRP1) complexed with peptidoglycan (PGN) is one of several described TREM-1 ligands whose engagement with the TREM-1 receptor triggers the production of inflammatory cytokines (e.g. TNFα) and was used in this assay. To complex the proteins, cynomolgus monkey PGLYRP1 (Creative Biomart) was combined with soluble PGN derived from E. coli (InvivoGen) in a 1.25:2 ratio and incubated for 45 minutes at 37° C. Complexed PGLYRP1/PGN was added to PBMC/antibody and incubated overnight at 37° C. The following day, cell media was collected and assayed for TNFα by Cyno TNFα Alphalisa proximity assay (Perkin Elmer). Light emission at 615 nm was measured on an Envision 2103 multilabel plate reader. IC50 values were calculated using GraphPad Prism (v8.4.3) and are presented for two separate assays in Table 15.

TABLE 15
TREM-1 Antibody Inhibition of Ligand Mediated Signaling
in Cyno PBMCs as Measured by TNFα Release

| | Ab IC50-Cyno PBMC | Ab IC50-Cyno PBMC |
|---|---|---|
| Antibody ID | (Trial 1) pM | (Trial 2) pM |
| 57C10 | 153.8 | 28.4 |
| 30H2 | 3768 | no inhibition |
| 61B12 | 46.9 | 17.4 |
| 61G5 | no inhibition | 2338 |
| 63F8 | 22.6 | 16.1 |
| 64D7 | 6939 | 780.5 |
| 34D1 | 100.1 | 16.2 |
| 44A5 | 57.9 | 9.1 |
| 46H7 | 95.1 | 9.7 |
| 49A2 | 731.5 | 265.7 |
| 66B8 | 113.9 | 23.2 |
| 50A12 | 2005 | nd |
| 57F5 | no inhibition | nd |
| 3E12 | no inhibition | no inhibition |

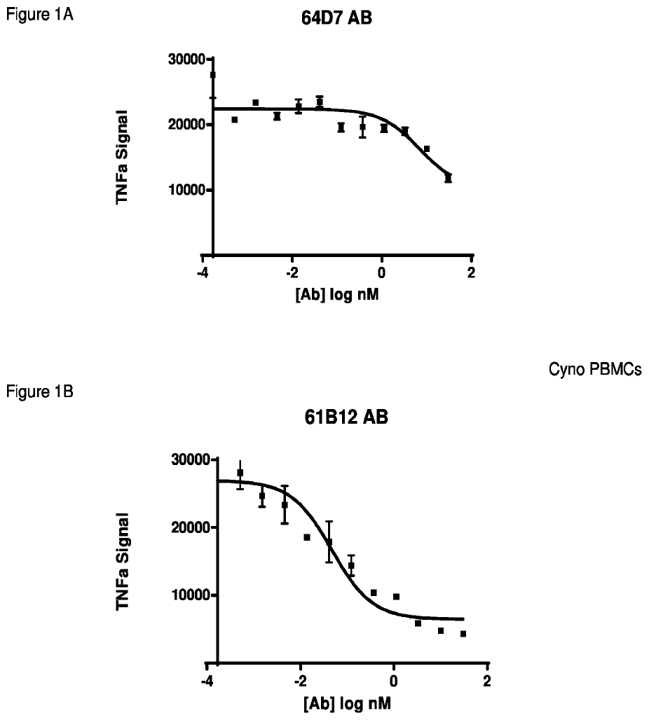

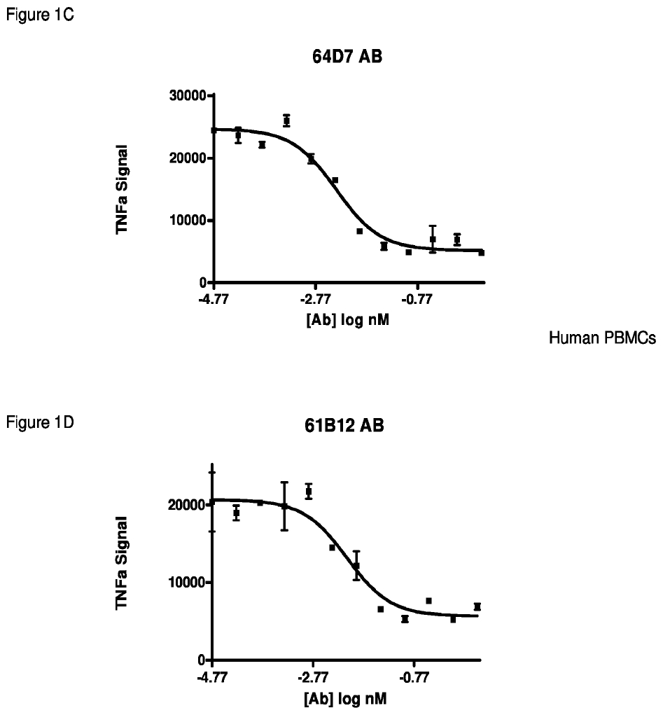

These results show that the TREM-1 antibodies bind cyno TREM-1, and importantly inhibit ligand-induced TREM-1 activation in cyno primary cells (PBMCs). Additionally, FIG. 1 illustrates TREM1 antibodies inhibition of PGLYRP1/PGN mediated TREM1 signaling in both cyno and human PBMCs.

### Example 5—Efficacy of TREM-1 Fabs Blocking Spleen Tyrosine Kinase (SYK) Phosphorylation in a Human TREM-1/Dap12-HEK293 Overexpressing Cell Line

TREM-1 Fabs were assayed for the ability to block signaling by the inhibition of ligand binding to the TREM-1 receptor in a cell line overexpressing human TREM-1/Dap12 and IC50s determined. Phosphorylation of Spleen Tyrosine Kinase (SYK) is an early step in the TREM-1 signaling cascade and in this assay is used as a measure of TREM-1 signaling. HEK293 cells that overexpress human TREM-1 and its obligate adaptor protein DNAX activation protein of 12 kDa (Dap12) were seeded in CellBIND plates (Corning) at 50K/well in complete media (DMEM/10% FBS) and allowed to attach overnight. Bivalent TREM-1 antibodies were unable to inhibit signaling in this system, likely due to a technical artefact of a high abundance of TREM-1 receptors in these cells and the ability of bivalent antibodies to crosslink them resulting in ligand-independent receptor agonism. Therefore, monovalent Fabs were used as surrogates for full antibodies in this assay. Fourteen anti-TREM-1 Fabs were diluted (3× serial dilution) in complete media to generate final antibody concentrations ranging from 0.00017 nM to 30 nM. Fabs were pre-incubated with TREM-1/Dap12-HEK293s for 30 minutes before addition of the TREM-1 ligand-complexed human PGLYRP1 (R&D Systems) and soluble PGN from E. coli (InvivoGen). To complex the PGLYRP1 and PGN, the proteins were combined in a 1:2 ratio and incubated at 37° C. for 45 minutes before adding to the cells/Fabs. Following a one hour room temperature incubation, the cells were lysed and the amount of phosphorylated SYK in the cell lysate was measured using the pSYK ALPHALISA® SUREFIRE® Ultra™ p-SYK (Tyr525/526) assay (Perkin Elmer). Light emission at 615 nm was measured on an Envision 2103 multilabel plate reader. IC50 values were calculated using GraphPad Prism (v8.4.3) and are presented for two separate assays as shown in Table 16.

TABLE 16
Antibody Inhibition of Ligand Mediated Signaling
in a Cell Line Overexpressing Human TREM-1/DAP12
As Measured by SYK phosphorylation

| | Fab IC50-pSYK Assay | Fab IC50-pSYK Assay |
|---|---|---|
| Fab ID | (Trial 1) pM | (Trial 2) pM |
| 57C10 Fab | 273.4 | 582.9 |
| 30H2 Fab | 467.2 | 374.5 |
| 61B12 Fab | 112.0 | 568.8 |
| 61G5 Fab | 387.0 | 186.4 |
| 63F8 Fab | 45.2 | 38.0 |
| 64D7 Fab | 229.1 | 70.0 |
| 34D1 Fab | 313.8 | 309.2 |
| 44A5 Fab | 188.9 | 325.0 |
| 46H7 Fab | 43.3 | 116.9 |
| 49A2 Fab | 1175 | 786.9 |
| 66B8 Fab | 4662 | nd |
| 50A12 Fab | 2850 | nd |
| 57F5 Fab | no inhibition | nd |
| 3E12 Fab | no inhibition | nd |

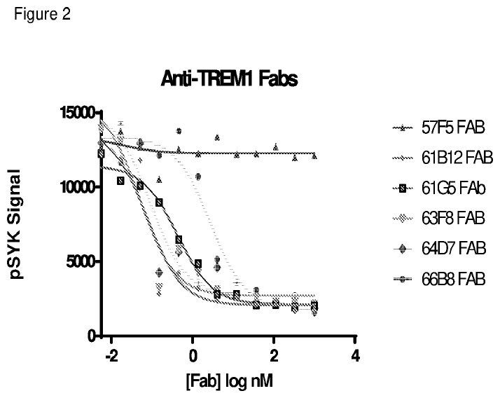

FIG. 2 is a graph showing that anti-TREM1 Fabs inhibit PGLYRP1/PGN-mediated SYK phosphorylation in TREM1/DAP12-HEK293 cells.

### Example 6—In Vivo Assessment of TREM-1 Antibody in Cardiovascular Disease

Both acute and disease models are used to establish that blocking TREM-1 mediated signaling will reduce proinflammatory cytokine/chemokine release (acute response) and result in improved disease outcomes for atherosclerosis (Ath) and myocardial infarction (MI).

### Acute Models:

### Lipopolysaccharide (LPS)-Induced Inflammation Model

To determine whether blockade of TREM-1 by mouse TREM-1 extracellular domain/Fc (muTREM1-Fc), results in a reduction in the release of proinflammatory cytokines in mice that have been systemically challenged with lipopolysaccharide (LPS) an LPS-Induced Inflammation Model is used. Briefly, female C57Bl/6 mice (10-12 weeks) are randomly grouped (n=6 mice/treatment) and treated intraperitoneally (i.p.) with either 3, 10 or 30 mg/kg (mpk) of muTREM1-Fc or 30 mpk of mulgG1 (control). One hour later, 100 μg/animal of LPS from E. coli (055:B5) will be administered (i.p.) to the mice. Blood samples will be collected at 0, 1, 3, 6, and 24 hours. Serum levels of proinflammatory cytokines and chemokines (IFN-γ, IL-1β, IL-2, IL-4, IL-5, IL-6, IL-10, IL-12p70, KC/GRO, TNF-α) are measured using the V-PLEX Proinflammatory Panel 1 Mouse Kit (Meso Scale Discovery). Soluble TREM-1 (sTREM-1) levels are measured by ELISA (R&D Systems). In addition to muTREM1-Fc analysis in this model, anti-mouse TREM-1 antibodies can be characterized.

### TREM-1 Ligand Mediated Inflammation Model

Peptidoglycan recognition protein 1 (PGLYRP1) has been identified as a TREM-1 ligand (Read et al., 2015). PGLYRP1 alone or complexed with its endogenous binding target (bacterial peptidoglycan-PGN) is used to initiate an inflammatory response in C57B1I/6 mice in a dose range of 0.5 to 3 mpk (PGLYRP1) and 0.5 to 5 mpk (PGN). Prior to administration of PGLYRP1 or PGLYRP1/PGN, the mice are treated with anti-mouse TREM-1 antibodies, control antibodies or muTREM1-Fc (dose range 1-30 mpk). Given the relatively low expression of TREM-1 in mice, LPS, a toll-like receptor 4 ligand known to upregulate expression of TREM-1 (Zeng et al., 2007), may be administered 24 hours before treatment with antibody/TREM1-Fc and TREM-1 ligand. Blood samples are collected at 0, 1, 3, 6, and 24 hours. Serum levels of proinflammatory cytokines and chemokines (IFN-γ, IL-1β, IL-2, IL-4, IL-5, IL-6, IL-10, IL-12p70, KC/GRO, TNF-α) are measured using the V-PLEX Proinflammatory Panel 1 Mouse Kit (Meso Scale Discovery). sTREM-1 levels will be measured by ELISA (R&D Systems).

### NSG Humanized Mouse Model

NOD scid gamma (NSG) mice (Jackson Labs) are severely immunodeficient mice that can be readily engrafted with human hematopoietic stem cells which allows for the establishment of a functional, humanized immune system in a mouse model. Utilizing this humanized mouse model, an inflammatory response is initiated either by LPS treatment, TREM-1 ligand treatment or both as described above. Prior to initiating the inflammatory response, mice are treated with either anti-human TREM-1 or isotype control antibodies (dose range 1-30 mpk). Blood samples will be collected at 0, 1, 3, 6, and 24 hours. Serum levels of proinflammatory cytokines and chemokines (IFN-γ, IL-1β, IL-2, IL-4, IL-6, IL-8, IL-10, IL-12p70, IL-13, TNF-α) are measured using the V-PLEX Proinflammatory Panel 1 Human Kit (Meso Scale Discovery). sTREM-1 levels will be measured by ELISA (R&D Systems).

### Cynomolgus Monkey Model

Cynomolgus (Macaca fascicularis) monkeys are treated with anti-human (cross reactive with Cynomolgus monkey) TREM-1 antibodies in an LPS challenge model. Adult male Cynomolgus monkeys are randomized to receive an intravenous (i.v.) or subcutaneous (s.c.) dose of either TREM-1 antibody (dose range 1-30 mpk) or isotype control after administration of an i.v. bolus of LPS (10 ug/kg of body weight). An additional control group of monkeys receives only vehicle (no antibody/no LPS). Vital signs are monitored and blood samples collected at 0, 1, 2, 4, and 8 hours for clinical chemistries, leukocyte count, coagulation parameters, soluble TREM-1 and cytokine plasma concentrations.

### Disease Models:

ApoE−/−, LDLR−/−, A20 haploinsufficient/ApoE−/−

Mouse models of atherosclerosis (e.g. ApoE−/− or LDLR−/− mice on high cholesterol high fat diet for 12-16 weeks) are used to determine if treatment with anti-mouse TREM-1 antibodies modifies disease outcomes. Mice are randomized and cohorts are administered s.c. doses (ranging from 1-30 mpk) of either control antibody or anti-mouse TREM-1 antibody weekly during the high cholesterol diet feeding phase. Blood is collected at various timepoints and analyzed for cytokine/chemokine levels, soluble TREM-1 levels and myeloid cell subsets. Upon conclusion of the experiment, whole aortas will be collected and surface lesion area quantified by en face staining using oil red 0 or Sudan IV. In addition, sections at the aortic root are obtained and stained with oil red 0 to quantify lesion area, anti-CD68 antibody staining to quantify macrophage content, and anti-smooth muscle actin (SMA) antibody staining to quantify smooth muscle cell content as well as other stains. Another useful model is the A20 haploinsufficient/ApoE−/− mice which demonstrate increased levels of NF-kb driven proatherosclerotic/proinflammatory target genes resulting in increased atherosclerosis susceptibility (Wolfrum et al., 2007). If no suitable anti-mouse TREM-1 surrogate antibody is identified, a human TREM-1 knock-in mouse in an ApoE−/− background will be generated. This would allow testing of the anti-human TREM-1 antibodies in a mouse model of atherosclerosis.

### NSG Humanized Mouse Model

Should the NSG mice prove useful in the acute setting, this could be developed into a disease model by siRNA knockdown of ApoE, Ldlr or both. Randomized cohorts of NSG/siRNA treated mice on high fat high cholesterol diets for 12-16 weeks will be administered s.c. doses (ranging from 1-30 mpk) of either control antibody or anti-human TREM-1 antibody weekly during the high cholesterol diet feeding phase. Blood is collected at various timepoints and analyzed for cytokine/chemokine levels, soluble TREM-1 levels and myeloid cell subsets. Upon conclusion of the experiment, whole aortas are collected and surface lesion area quantified by en face staining and aortic root sections stained as described above.

### Rodent Acute Myocardial Infarction (AMI) Models

Rodent models of MI are useful to test the anti-mouse TREM-1 antibodies and/or muTREM1-Fc. Briefly, AMI is induced in either mice or rats by permanent ligation of the left anterior descending coronary artery (LAD). Following this procedure, rodents (rats=Sprague Dawley, mice=C57Bl/6) will be randomized and receive an s.c. dose (1-30 mpk) of either anti-mouse TREM-1 or isotype control antibody weekly for 2-8 weeks post ligation surgery. Cardiac function (e.g. ejection fraction, end diastolic volume, end systolic volumes and cardiac output) is assessed by noninvasive echocardiography at defined times throughout the study period. Terminal cardiac function and contractility will be measured with pressure volume loops. Blood samples are drawn at regular intervals to measure blood cell counts, liver and kidney enzymes, troponin, soluble TREM-1 and inflammatory markers (cytokines, hsCRP). At the conclusion of the study, rodents are euthanized and infarct size in relation to the area at risk (IS/AAR) will be determined by ex vivo histological staining. A similar study can be performed in which ligation of the LAD occurs for 90 minutes followed by removal of the ligation, the so called ischemia/reperfusion (I/R) model. The treatment scheme and analysis would be the same as described above.

### Mini Pig MI Model

Antibodies that may cross react with pig TREM-1, are tested in a mini-pig AMI model. Briefly, AMI is induced in minipigs (Sus scrofa domestic) by inflation of an angioplasty balloon in the proximal left anterior descending artery (LAD) for one hour. Animals are randomized to receive s.c. doses (1-30 mpk) of either a TREM-1 or isotype control antibody weekly for 2-4 weeks after surgery. Following administration of the initial dose of antibody at the time of surgery, the angioplasty balloon is deflated. Hemodynamic parameters including heart rate, mean arterial pressure, mean pulmonary artery pressure, cardiac output, cardiac index and mixed venous oxygen saturation will be monitored throughout the study period. Pressure-conductance catheter based parameters including end-diastolic and end-systolic volumes of the left ventricle, ejection fraction, maximum and minimum values of the first derivative of ventricular pressure (dP/dtmax, dP/dtmin), time constant of left ventricle pressure decay and stroke work will be monitored regularly throughout the study period. Pressure/volume data with vena cava occlusion is also collected, including end-systolic and end-diastolic pressure volume relationships, maximum ventricular elastance, arterial elastance, dp/dtmax to end diastolic volume relationship, pre-load recruitable stroke work and pressure volume area. Blood samples are drawn at regular intervals to measure blood gases, blood cell counts, blood lactates, liver and kidney enzymes, troponin, soluble TREM-1 and inflammatory markers (cytokines, hsCRP). At the conclusion of the study, pigs are euthanized and infarct size in relation to the area at risk (IS/AAR) will be determined by ex vivo histological staining.

It is hypothesized that administration of TREM-1 antibodies that block signaling through the receptor will down regulate inflammatory responses in the cardiac models and treat cardiovascular disease by reducing one or more symptoms of cardiovascular disease (e.g., atherosclerosis or myocardial infarction), such as inflammatory cell migration to the site of injury, infiltration of myeloid cells into cardiac tissue, inflammatory cytokines in the microenvironment, tissue damage, reduction in foam cell formation, reduction in necrotic core size, reduction in scar formation, reduction in endothelial cell dysfunction, and/or reduction in thrombus formation.

### Example 7—Genetic Ablation of TREM-1 Resulted in Decreased Levels of Serum Peptidoglycan Recognition Protein 1 (PGLYRP1) Following LPS Stimulation

TREM-1 knockout (C57Bl/6 background) and age/background matched mice (n=6) were injected (intraperitoneal) with 100 μg/animal Lipopolysaccharide (LPS) from E.Coli (055:B5) (Sigma-Aldrich). Twenty microliters of whole blood were drawn at 0, 1, 3, 6 and 24 hours post LPS administration. Serum from the two treatment groups (WT, TREM-1 KO) was pooled for each time point and PGLYRP1 concentrations were measured by ELISA (LSBio). Optical density at 450 nM was measured on a Molecular Devices SpectraMax Plus³⁸⁴ Spectrophotometer.

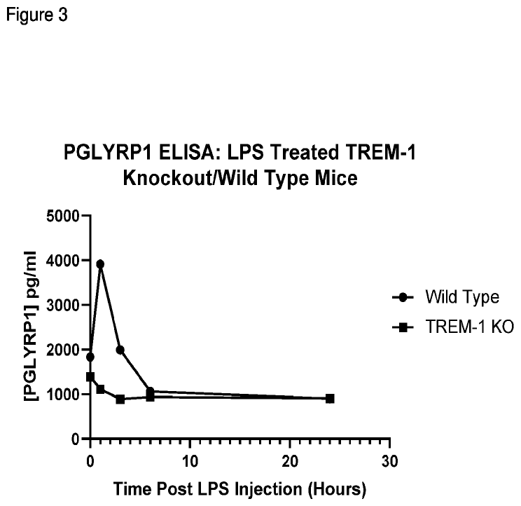

FIG. 3 is a graph showing a spike at 1 hour in TREM-1 ligand PGLYRP1 in wild-type but not TREM-1 knockout mice following administration of LPS. This data suggests that TREM-1 is involved in the regulation of PGLYRP1 and that an antibody targeting TREM-1 could both block ligand binding to the receptor and the subsequent availability of the PGLYRP1 ligand. Blocking the increase in PYGLYRP1 using a TREM-1 antibody may prevent a subsequent PYGLRP1-mediated inflammatory and cardiovascular event.

Any single embodiment herein may be supplemented with one or more element from any one or more other embodiment herein.

It is understood, therefore, that this invention is not limited to the particular embodiments disclosed, but is intended to cover all modifications which are within the spirit and scope of the invention as defined by the appended claims; the above description; the following numbered paragraphs, and/or shown in the attached drawings.

Numerous modifications and variations in the invention as set forth in the above illustrative examples are expected to occur to those skilled in the art. Consequently only such limitations as appear in the appended claims should be placed on the invention.

What is claimed is:

1. A method of treating cardiovascular disease comprising administering to a subject in need thereof a therapeutically effective amount of an antigen binding protein that binds to TrigGering Receptor Expressed on Myeloid cells 1 (TREM-1);
the antigen binding protein comprising
a. a light chain variable domain comprising:
i. a light chain CDR1 comprising an amino acid sequence selected from SEQ ID NOS: 10, 30, 50, 70, 90, 110, 130, 150, 170, 190, 210, 230, 250, 270, and 544;
ii. a light chain CDR2 comprising an amino acid sequence selected from SEQ ID NOS: 11, 31, 51, 71, 91, 111, 131, 151, 171, 191, 211, 231, 251, 271, and 545;
iii. a light chain CDR3 comprising an amino acid sequence selected from SEQ ID NOS: 12, 32, 52, 72, 92, 112, 132, 152, 172, 192, 212, 232, 252, 272, and 546; and
b. a heavy chain variable domain comprising:
i. a heavy chain CDR1 comprising an amino acid sequence selected from SEQ ID NOS: 16, 36, 56, 76, 96, 116, 136, 156, 176, 196, 216, 236, 256, 276, and 550;
ii. a heavy chain CDR2 comprising an amino acid sequence selected from SEQ ID NOS: 17, 37, 57, 77, 97, 117, 137, 157, 177, 197, 217, 237, 257, 277, 297 and 551; and
iii. a heavy chain CDR3 comprising an amino acid sequence selected from SEQ ID NOS: 18, 38, 58, 78, 98, 118, 138, 158, 178, 198, 218, 238, 258, 278, 298 and 552.

2. The method of claim 1, wherein the antigen binding protein comprises:
a. the light chain CDR1 sequence set out in SEQ ID NO: 10, 30, 50, 90, 130, 150, or 270;
b. the light chain CDR2 sequence set out in SEQ ID NOS: 11, 31, 51, 91, 131, 151, or 271;
c. the light chain CDR3 sequence set out in SEQ ID NO 12, 32, 52, 92, 132, 152, or 272;
d. the heavy chain CDR1 sequence set out in SEQ ID NO: 16, 36, 56, 96, 136, 156, and 276;
e. the heavy chain CDR2 sequence set out in SEQ ID NO: 17, 37, 57, 97, 137, 157, and 277; and
f. the heavy chain CDR3 sequence set out in SEQ ID NO: 18, 38, 58, 98, 138, and 278.

3. The method of claim 1 or 2, wherein the antigen binding protein comprises:
a. the light chain CDR1 sequence set out in SEQ ID NO: 30 or 90;
b. the light chain CDR2 sequence set out in SEQ ID NO: 31 or 91;
c. the light chain CDR3 sequence set out in SEQ ID NO: 32 or 92;
d. the heavy chain CDR1 sequence set out in SEQ ID NO: 36 or 96;
e. the heavy chain CDR2 sequence set out in SEQ ID NO: 37 or 97; and
f. the heavy chain CDR3 sequence set out in SEQ ID NOS: 38 or 98

4. The method of any one of claims 1 to 3 wherein the antigen-binding protein comprises:
i) SEQ ID NO: 10 (LCDR1), SEQ ID NO: 11 (LCDR2), SEQ ID NO: 12 (LCDR3), SEQ ID NO: 16 (HCDR1), SEQ ID NO: 17 (HCDR2) and SEQ ID NO: 18 (HCDR3);
ii) SEQ ID NO: 30 (LCDR1), SEQ ID NO: 31 (LCDR2), SEQ ID NO: 32 (LCDR3), SEQ ID NO: 36 (HCDR1), SEQ ID NO: 37 (HCDR2) and SEQ ID NO: 38 (HCDR3);
iii) SEQ ID NO: 50 (LCDR1), SEQ ID NO: 51 (LCDR2), SEQ ID NO: 52 (LCDR3), SEQ ID NO: 56 (HCDR1), SEQ ID NO: 57 (HCDR2) and SEQ ID NO: 58 (HCDR3);
iv) SEQ ID NO: 70 (LCDR1), SEQ ID NO: 71 (LCDR2), SEQ ID NO: 72 (LCDR3), SEQ ID NO: 76 (HCDR1), SEQ ID NO: 77 (HCDR2) and SEQ ID NO: 78 (HCDR3);
v) SEQ ID NO: 90 (LCDR1), SEQ ID NO: 91 (LCDR2), SEQ ID NO: 92 (LCDR3), SEQ ID NO: 96 (HCDR1), SEQ ID NO: 97 (HCDR2) and SEQ ID NO: 98 (HCDR3);
vi) SEQ ID NO: 110 (LCDR1), SEQ ID NO: 111 (LCDR2), SEQ ID NO: 112 (LCDR3), SEQ ID NO: 116 (HCDR1), SEQ ID NO: 117 (HCDR2) and SEQ ID NO: 118 (HCDR3);
vii) SEQ ID NO: 130 (LCDR1), SEQ ID NO: 131 (LCDR2), SEQ ID NO: 132 (LCDR3), SEQ ID NO: 136 (HCDR1), SEQ ID NO: 137 (HCDR2) and SEQ ID NO: 138 (HCDR3);
viii) SEQ ID NO: 150 (LCDR1), SEQ ID NO: 151 (LCDR2), SEQ ID NO: 152 (LCDR3), SEQ ID NO: 156 (HCDR1), SEQ ID NO: 157 (HCDR2) and SEQ ID NO: 158 (HCDR3);
ix) SEQ ID NO: 170 (LCDR1), SEQ ID NO: 171 (LCDR2), SEQ ID NO: 172 (LCDR3), SEQ ID NO: 176 (HCDR1), SEQ ID NO: 177 (HCDR2) and SEQ ID NO: 178 (HCDR3);
x) SEQ ID NO: 190 (LCDR1), SEQ ID NO: 191 (LCDR2), SEQ ID NO: 192 (LCDR3), SEQ ID NO: 196 (HCDR1), SEQ ID NO: 197 (HCDR2) and SEQ ID NO: 198 (HCDR3);
xi) SEQ ID NO: 210 (LCDR1), SEQ ID NO: 211 (LCDR2), SEQ ID NO: 212 (LCDR3), SEQ ID NO: 216 (HCDR1), SEQ ID NO: 217 (HCDR2) and SEQ ID NO: 218 (HCDR3);
xii) SEQ ID NO: 230 (LCDR1), SEQ ID NO: 231 (LCDR2), SEQ ID NO: 232 (LCDR3), SEQ ID NO: 236 (HCDR1), SEQ ID NO: 237 (HCDR2) and SEQ ID NO: 238 (HCDR3);
xiii) SEQ ID NO: 250 (LCDR1), SEQ ID NO: 251 (LCDR2), SEQ ID NO: 252 (LCDR3), SEQ ID NO: 256 (HCDR1), SEQ ID NO: 257 (HCDR2) and SEQ ID NO: 258 (HCDR3);
xiv) SEQ ID NO: 270 (LCDR1), SEQ ID NO: 271 (LCDR2), SEQ ID NO: 272 (LCDR3), SEQ ID NO: 276 (HCDR1), SEQ ID NO: 277 (HCDR2) and SEQ ID NO: 278 (HCDR3); or
xv) SEQ ID NO: 544 (LCDR1), SEQ ID NO: 545 (LCDR2), SEQ ID NO: 546 (LCDR3), SEQ ID NO: 550 (HCDR1), SEQ ID NO: 551 (HCDR2) and SEQ ID NO: 552 (HCDR3).

5. The method of any one of claims 1 to 4, wherein the antigen-binding protein comprises a set of CDR selected from:
i) SEQ ID NO: 10 (LCDR1), SEQ ID NO: 11 (LCDR2), SEQ ID NO: 12 (LCDR3), SEQ ID NO: 16 (HCDR1), SEQ ID NO: 17 (HCDR2) and SEQ ID NO: 18 (HCDR3);
ii) SEQ ID NO: 30 (LCDR1), SEQ ID NO: 31 (LCDR2), SEQ ID NO: 32 (LCDR3), SEQ ID NO: 36 (HCDR1), SEQ ID NO: 37 (HCDR2) and SEQ ID NO: 38 (HCDR3);
iii) SEQ ID NO: 50 (LCDR1), SEQ ID NO: 51 (LCDR2), SEQ ID NO: 52 (LCDR3), SEQ ID NO: 56 (HCDR1), SEQ ID NO: 57 (HCDR2) and SEQ ID NO: 58 (HCDR3);
iv) SEQ ID NO: 90 (LCDR1), SEQ ID NO: 91 (LCDR2), SEQ ID NO: 92 (LCDR3), SEQ ID NO: 96 (HCDR1), SEQ ID NO: 97 (HCDR2) and SEQ ID NO: 98 (HCDR3);
v) SEQ ID NO: 130 (LCDR1), SEQ ID NO: 131 (LCDR2), SEQ ID NO: 132 (LCDR3), SEQ ID NO: 136 (HCDR1), SEQ ID NO: 137 (HCDR2) and SEQ ID NO: 138 (HCDR3);
vi) SEQ ID NO: 150 (LCDR1), SEQ ID NO: 151 (LCDR2), SEQ ID NO: 152 (LCDR3), SEQ ID NO: 156 (HCDR1), SEQ ID NO: 157 (HCDR2) and SEQ ID NO: 158 (HCDR3); or
vii) SEQ ID NO: 270 (LCDR1), SEQ ID NO: 271 (LCDR2), SEQ ID NO: 272 (LCDR3), SEQ ID NO: 276 (HCDR1), SEQ ID NO: 277 (HCDR2) and SEQ ID NO: 278 (HCDR3).

6. The method any one of claims 1 to 5 wherein of antigen-binding protein comprises:
a. a light chain comprising the amino acid sequences SEQ ID NO: 30 (LCDR1), SEQ ID NO: 31 (LCDR2), SEQ ID NO: 32 (LCDR3), and a heavy chain comprising the amino acid sequences SEQ ID NO: 36 (HCDR1), SEQ ID NO: 37 (HCDR2) and SEQ ID NO: 38 (HCDR3); or
b. a light chain comprising the amino acid sequences SEQ ID NO: 90 (LCDR1), SEQ ID NO: 91 (LCDR2), SEQ ID NO: 92 (LCDR3), and a heavy chain comprising the amino acid sequences SEQ ID NO: 96 (HCDR1), SEQ ID NO: 97 (HCDR2) and SEQ ID NO: 98 (HCDR3).

7. The method of any one of claims 1 to 6, wherein the antigen-binding protein comprises:
a. a light chain variable domain comprising an amino acid sequence selected from the group consisting of:
i. a sequence at least 80% identical to an amino acid sequence selected from SEQ ID NOS: 21, 41, 61, 81, 101, 121, 141, 161, 181, 201, 221, 241, 261, 281 and 539;
ii. a sequence encoded by a polynucleotide sequence that is at least 80% identical to an amino acid sequence selected from SEQ ID NOS: 21, 41, 61, 81, 101, 121, 141,161, 181, 201, 221, 241, 261, 281 and 539; or
iii. a sequence encoded by a polynucleotide that hybridizes under moderately stringent conditions to the complement of a polynucleotide consisting of a nucleic acid sequence selected from SEQ ID NOS: 19, 39, 59, 79, 99, 119, 139, 159, 179, 199, 219, 239, 259, 279 and 537; and
b. a heavy chain variable domain comprising an amino acid sequence selected from the group consisting of:
i. a sequence that is at least 80% identical to an amino acid sequence selected from SEQ ID NOS: 22, 42, 62, 82, 102, 122, 142, 162, 182, 202, 222, 242, 262, 282, and 540;
ii. a sequence encoded by a polynucleotide sequence that is at least 80% identical to an amino acid sequence selected from SEQ ID NOS: 22, 42, 62, 82, 102, 122, 142, 162, 182, 202, 222, 242, 262, 282, and 540; or
iii. a sequence encoded by a polynucleotide that hybridizes under moderately stringent conditions to the complement of a polynucleotide consisting of a nucleic sequence selected from SEQ ID NOS: 20, 40, 60, 80, 100, 120, 140, 160, 180, 200, 220, 240, 260, 280, and 2184.

8. The method of any one of claims 1 to 7, wherein the antigen-binding protein comprises an amino acid sequence at least 90% identical to a heavy chain variable region amino acid sequence selected from the group consisting of SEQ ID NOS: 22, 42, 62, 82, 102, 122, 142, 162, 182, 202, 222, 242, 262, 282, and 540.

9. The method of any one of claims 1 to 8, wherein the antigen binding protein comprises an amino acid sequence at least 90% identical to a heavy chain variable region amino acid sequence selected from the group consisting of SEQ ID NOs: 22, 42, 62, 102, 142, 162, and 282.

10. The method of any one of claims 1 to 9, wherein the antigen binding protein comprises an amino acid sequence at least 90% identical to a heavy chain variable region amino acid sequence selected from the group consisting of SEQ ID NOs: 42 and 102.

11. The method of any one of claims 1 to 10, wherein the antigen-binding protein comprises an amino acid sequence at least 90% identical to a light chain variable region amino acid sequence selected from the group consisting of SEQ ID NOS: 21, 41, 61, 81, 101, 121, 141, 161, 181, 201, 221, 241, 261, 281 and 539.

12. The method of any one of claims 1 to 11, wherein the antigen binding protein comprises an amino acid sequence at least 90% identical to a light chain variable region amino acid sequence selected from the group consisting of SEQ ID NOS: 21, 41, 61, 101, 141, 161, and 281.

13. The method of any one of claims 1 to 12, wherein the antigen binding protein comprises an amino acid sequence at least 90% identical to a light chain variable region amino acid sequence selected from the group consisting of SEQ ID NOS: 41 and 101.

14. The method of any one of claims 1 to 13, wherein the antigen binding protein comprises:
i) a light chain variable domain sequence set out in SEQ ID NO: 21 and a heavy chain variable domain sequence set out in SEQ ID NO: 22;
ii) a light chain variable domain sequence set out in SEQ ID NO: 41 and a heavy chain variable domain sequence set out in SEQ ID NO: 42;
iii) a light chain variable domain sequence set out in SEQ ID NO: 61 and a heavy chain variable domain sequence set out in SEQ ID NO: 62;
iv) a light chain variable domain sequence set out in SEQ ID NO: 81 and a heavy chain variable domain sequence set out in SEQ ID NO: 82;
v) a light chain variable domain sequence set out in SEQ ID NO: 101 and a heavy chain variable domain sequence set out in SEQ ID NO: 102;
vi) a light chain variable domain sequence set out in SEQ ID NO: 121 and a heavy chain variable domain sequence set out in SEQ ID NO: 122;
vii) a light chain variable domain sequence set out in SEQ ID NO: 141 and a heavy chain variable domain sequence set out in SEQ ID NO: 142;
viii) a light chain variable domain sequence set out in SEQ ID NO: 161 and a heavy chain variable domain sequence set out in SEQ ID NO: 162;
ix) a light chain variable domain sequence set out in SEQ ID NO: 181 and a heavy chain variable domain sequence set out in SEQ ID NO: 182;
x) a light chain variable domain set out in SEQ ID NO: 201 and a heavy chain variable domain set out in SEQ ID NO: 202;
xi) a light chain variable domain sequence set out in SEQ ID NO: 221 and a heavy chain variable domain sequence set out in SEQ ID NO: 222;
xii) a light chain variable domain sequence set out in SEQ ID NO: 241 and a heavy chain variable domain sequence set out in SEQ ID NO: 242;
xiii) a light chain variable domain sequence set out in SEQ ID NO: 261 and a heavy chain variable domain sequence set out in SEQ ID NO: 262;
xiv) a light chain variable domain sequence set out in SEQ ID NO: 281 and a heavy chain variable domain sequence set out in SEQ ID NO: 282; or
xv) a light chain variable domain sequence set out in SEQ ID NO: 539 and a heavy chain variable domain sequence set out in SEQ ID NO: 540.

15. The method of any one of claims 1 to 14, wherein the antigen binding protein comprises:
i) a light chain variable domain sequence set out in SEQ ID NO: 21 and a heavy chain variable domain set out in SEQ ID NO: 22;
ii) a light chain variable domain sequence set out in SEQ ID NO: 41 and a heavy chain variable domain sequence set out in SEQ ID NO: 42;
iii) a light chain variable domain sequence set out in SEQ ID NO: 61 and a heavy chain variable domain sequence set out in SEQ ID NO: 62;
iv) a light chain variable domain sequence set out in SEQ ID NO: 101 and a heavy chain variable domain sequence set out in SEQ ID NO: 82;
v) a light chain variable domain sequence set out in SEQ ID NO: 141 and a heavy chain variable domain sequence set out in SEQ ID NO: 142;
vi) a light chain variable domain sequence set out in SEQ ID NO: 161 and a heavy chain variable domain sequence set out in SEQ ID NO: 162;
vii) a light chain variable domain sequence set out in SEQ ID NO: 281 and a heavy chain variable domain sequence set out in SEQ ID NO: 282.

16. The method of any one of claims 1 to 15, wherein the antigen binding protein comprises:
a light chain variable domain sequence set out in SEQ ID NO: 41 and a heavy chain variable domain sequence set out in SEQ ID NO: 42; or a light chain variable domain sequence set out in SEQ ID NO: 101 and a heavy chain variable domain sequence set out in SEQ ID NO: 102.

17. The method of any one of claims 1 to 16, wherein one or more heavy chain framework amino acids of the anti-antigen-binding protein are replaced with corresponding amino acid(s) from another human antibody amino acid sequence.

18. The method of any one of claims 1 to 17, wherein one or more light chain framework amino acids of the antigen-binding protein are replaced with corresponding amino acid(s) from another human antibody amino acid sequence.

19. The method of any one of claims 1 to 18, wherein the heavy chain comprises a constant region selected from heavy chain constant regions of an IgG, IgM, IgA, IgD, IgE, fragments thereof, combinations thereof, and modifications thereof in which one to ten heavy chain framework amino acids are replaced with corresponding amino acid(s) from another human antibody amino acid sequence.

20. A method of treating cardiovascular disease comprising administering to a subject in need thereof a therapeutically effective amount of an antigen binding protein that competes for binding to TREM-1 with an antigen binding protein of any one of claims 1 to 19.

21. The method of any one of claims 1 to 20, wherein the antigen-binding protein is selected from the group consisting of a human antibody, a humanized antibody, a chimeric antibody, a monoclonal antibody, a recombinant antibody, a Fab, a F(ab′)2, a Fab2, a monovalent IgG, an scFv, an scFv-Fc, an IgG1 antibody, an IgG2 antibody, an IgG3 antibody, and an IgG4 antibody.

22. The method of any one of claims 1 to 21, wherein the antigen-binding protein is an IgG1 antibody.

23. The method of any one of claims 1 to 22, wherein the antigen-binding protein is a monovalent IgG.

24. The method of any one of claims 1 to 23, wherein the antigen-binding protein is a human antibody.

25. The method of any one of claims 1 to 24 wherein the antigen binding protein comprises a heavy chain amino acid sequence at least 90% identical to a sequence selected from SEQ ID NOS: 22, 42, 62, 82, 102, 122, 142, 162, 182, 202, 222, 242, 262, 282, and 540 and a light chain amino acid sequence at least 90% identical to a sequence selected from SEQ ID NOS: 21, 41, 61, 81, 101, 121, 141, 161, 181, 201, 221, 241, 261, 281 and 539.

26. The method of any one of claims 1 to 25 wherein the antigen-binding protein has a heavy chain amino acid sequence selected from SEQ ID NOS: 22, 42, 62, 82, 102, 122, 142, 162, 182, 202, 222, 242, 262, 282, and 540 and a light chain amino acid sequence selected from SEQ ID NOS: 21, 41, 61, 81, 101, 121, 141, 161, 181, 201, 221, 241, 261, 281 and 539.

27. The method of any one of claims 1 to 26, wherein the antigen-binding protein has a heavy chain amino acid sequence selected from SEQ ID NOS: 22, 42, 62, 102, 142, 162, and 282.

28. The method of any one of claims 1 to 27, wherein the antigen-binding protein has a heavy chain amino acid sequence selected from SEQ ID NOS: 42 and 102.

29. The method of any one of claims 1 to 27, wherein the antigen-binding protein has a light chain amino acid sequence selected from SEQ ID NOS: 21, 41, 61, 81, 101, 141, 161, and 281.

30. The method of any one of claims 1 to 27, wherein the antigen-binding protein has a light chain amino acid sequence selected from SEQ ID NOS: 41 and 101.

31. The method of any one of claims 1 to 30, wherein the antigen binding protein further comprises a pharmaceutically acceptable carrier.

32. The method of any one of claims 1-31, wherein the cardiovascular disease is selected from the group consisting of myocardial infarction, non-alcoholic fatty liver disease (NAFLD), non-alcoholic steatohepatitis (NASH), heart failure, stroke (ischemic and hemorrhagic), atherosclerosis, coronary artery disease, peripheral vascular disease (e.g. peripheral artery disease), vulnerable plaque, acute coronary syndrome, cerebrovascular disease, cerebrovascular atherosclerosis and obesity.

33. The method of claim any one of claims 1 to 32, wherein the cardiovascular disease is atherosclerosis.

34. The method of any one of claims 1 to 32, wherein the cardiovascular disease is myocardial infarction.

35. The method of any one of claims 1-34, wherein the treatment is administered intravenously or subcutaneously.

36. The method of any one of claims 1-35, wherein the treatment is administered once weekly, once every two weeks, once every three weeks, once every 4 weeks, once monthly, once every 3 months, once every six months, or once yearly.

37. The method of any one of claims 1-36, further comprising administering one or two additional therapeutic agents.

38. The method of claim 37, wherein the additional therapeutic agents are selected from the group consisting of one or more cholesterol-lowering agent, an agent that increases the expression of LDLR, statins (atorvastatin, cerivastatin, fluvastatin, lovastatin, mevastatin, pitavastatin, pravastatin, rosuvastatin, simvastatin), PCSK9 inhibitors (Repatha®, Praluent®, Leqvio®), nicotinic acid (Niacin) (NIACOR®, NIASPANG (slow release niacin), SLO-NIACIN®(slow release niacin), Fibric acid (LOPID® (gemfibrozil), TRICOR® (fenofibrate), Bile acid sequestrants (QUESTRAN® (cholestyramine), colesevelam (WELCHOL®), COLESTID® (colestipol), Cholesterol absorption inhibitors (Zetia (ezetimibe)), combination of nicotinic acid with statin (ADVICOR® (LOVASTATIN and NIASPAN®), combination of a statin with an absorption inhibitor (VYTORIN (ZOCOR® and ZETIA®) and/or lipid modifying agents like PCSK9 inhibitors, PPAR gamma agonists, PPAR alpha/gamma agonists, squalene synthase inhibitors, cholesterylester transfer protein (CETP) inhibitors, anti-hypertensives, anti-thrombotics (aspirin) anti-diabetic agents (such as sulphonyl ureas, insulin, GLP-1 analogs, DDPIV inhibitors, SGL2 inhibitors), ApoB modulators, MTP inhibitors, Corlanor® (ivabradine) I(f) current Inhibitor, omecamtiv mecarbil cardiac myosin activator, OLPASIRAN (AMG890) (siRNA) that lowers lipoprotein(a), AMG 594 cardiac troponin activator, AMG 609, AMG 171 (Growth Differential Factor 15 (GDF15) analog), AMG 133 (gastric inhibitory polypeptide receptor (GIPR) antagonist and glucagon-like peptide 1 (GLP-1) receptor agonist, and/or arteriosclerosis obliterans treatments.

39. The method of any one of claims 1 to 38, wherein the TREM-1 is human TREM-1 set out in SEQ ID NO: 2.

40. The method of any one of claims 1 to 39, wherein the administration reduces one or more symptoms of cardiovascular disease selected from the group consisting of inflammatory cell migration to the site of injury, infiltration of myeloid cells into cardiac tissue, inflammatory cytokines in the microenvironment, tissue damage, reduction in foam cell formation, reduction in necrotic core size, reduction in scar formation, reduction in endothelial cell dysfunction, and/or reduction in thrombus formation.

41. A composition comprising an antigen binding protein that binds to TrigGering Receptor Expressed on Myeloid cells 1 (TREM-1) for use in treating a cardiovascular disease, wherein the antigen binding protein comprises:
a. a light chain variable domain comprising:
i. a light chain CDR1 comprising an amino acid sequence selected from SEQ ID NOS: 10, 30, 50, 70, 90, 110, 130, 150, 170, 190, 210, 230, 250, 270 and 544;
ii. a light chain CDR2 comprising an amino acid sequence selected from SEQ ID NOS: 11, 31, 51, 71, 91, 111, 131, 151, 171, 191, 211, 231, 251, 271, and 545;
iii. a light chain CDR3 comprising an amino acid sequence selected from SEQ ID NOS: 12, 32, 52, 72, 92, 112, 132, 152, 172, 192, 212, 232, 252, 272 and 546; and
b. a heavy chain variable domain comprising:
i. a heavy chain CDR1 comprising an amino acid sequence selected from SEQ ID NOS: 16, 36, 56, 76, 96, 116, 136, 156, 176, 196, 216, 236, 256, 276, and 550;
ii. a heavy chain CDR2 comprising an amino acid sequence selected from SEQ ID NOS: 17, 37, 57, 77, 97, 117, 137, 157, 177, 197, 217, 237, 257, 277, and 551; and
iii. a heavy chain CDR3 comprising an amino acid sequence selected from SEQ ID NOS: 18, 38, 58, 78, 98, 118, 138, 158, 178, 198, 218, 238, 258, 278, and 552.

42. Use of a composition comprising antigen binding protein that binds to TrigGering Receptor Expressed on Myeloid cells 1 (TREM-1) in the preparation of a medicament for treating a cardiovascular disease, the antigen binding protein comprising
a. a light chain variable domain comprising:
i. a light chain CDR1 comprising an amino acid sequence selected from SEQ ID NOS: 10, 30, 50, 70, 90, 110, 130, 150, 170, 190, 210, 230, 250, 270 and 544;
ii. a light chain CDR2 comprising an amino acid sequence selected from SEQ ID NOS: 11, 31, 51, 71, 91, 111, 131, 151, 171, 191, 211, 231, 251, 271, and 545;
iii. a light chain CDR3 comprising an amino acid sequence selected from SEQ ID NOS: 12, 32, 52, 72, 92, 112, 132, 152, 172, 192, 212, 232, 252, 272 and 546; and
b. a heavy chain variable domain comprising:
i. a heavy chain CDR1 comprising an amino acid sequence selected from SEQ ID NOS: 16, 36, 56, 76, 96, 116, 136, 156, 176, 196, 216, 236, 256, 276, and 550;
ii. a heavy chain CDR2 comprising an amino acid sequence selected from SEQ ID NOS: 17, 37, 57, 77, 97, 117, 137, 157, 177, 197, 217, 237, 257, 277, and 551; and
iii. a heavy chain CDR3 comprising an amino acid sequence selected from SEQ ID NOS: 18, 38, 58, 78, 98, 118, 138, 158, 178, 198, 218, 238, 258, 278, and 552.

43. The use of claim 41 or 42 wherein the cardiovascular disease is selected from the group consisting of myocardial infarction, non-alcoholic fatty liver disease (NAFLD), non-alcoholic steatohepatitis (NASH), heart failure, stroke (ischemic and hemorrhagic), atherosclerosis, coronary artery disease, peripheral vascular disease (e.g. peripheral artery disease), vulnerable plaque, acute coronary syndrome, cerebrovascular disease, cerebrovascular atherosclerosis and obesity.

44. A method of treating cardiovascular disease comprising administering to a subject in need thereof a therapeutically effective amount of an antigen binding protein that binds to TrigGering Receptor Expressed on Myeloid cells 1 (TREM-1), the antigen binding protein comprising:
a. a light chain variable domain comprising:
i. a light chain CDR1 comprising an amino acid sequence X₁ASQSX₂X₃X₄NLA (SEQ ID NO: 553), wherein X₁ is R or Q, wherein X₂ is V or I, wherein X₃ is N or S, and wherein X₄ is S, H, I, V or A;
ii. a light chain CDR2 comprising an amino acid sequence GAX₁X₂RAT (SEQ ID NO: 554), wherein X₁ is S or Y, and wherein X₂ is T or I; and
iii. a light chain CDR3 comprising an amino acid sequence QX₁X₂X₃X₄X₅X₆PX₇T (SEQ ID NO: 555); wherein X₁ is Q, H or E, wherein X₂ is F or Y, wherein X₃ is K, Y or I, wherein X₄ is N, T, L, I, or M; wherein X₅ is W, F, H or Y, wherein X₆ is absent or P; wherein X₇ is W, N, Y, H or L; and
b. a heavy chain variable domain comprising:
i. a heavy chain CDR1 comprising an amino acid sequence X₁X₂X₃MX₄ (SEQ ID NO: 556), wherein X₁ is A, R, T or S, wherein X₂ is Y or N, wherein X₃ is A or W, and wherein X₄ is S or N;
ii. a heavy chain CDR2 comprising an amino acid sequence X₁X₂X₃X₄X₅X₆ X₇ X₈ X₉YYX₁₀ X₁₁X₁₂VKG (SEQ ID NO: 559), wherein X₁ is T, E, or S, wherein X₂ is absent or is M, V, or I, wherein X₃ is S, R or K, wherein X₄ is G or Q, wherein X₅ is S, D or H, wherein X₆ is G, S L, or A, wherein X₇ is S, G, or R, wherein X₈ is T, S, P or E, wherein X₉ is T or I, wherein X₁₀ is A or V, wherein X₁₁ is D or E, and wherein X₁₂ is S or A; and
iii. a heavy chain CDR3 comprising an amino acid sequence X₁X₂X₃X₄X₅X₆ X₇ F X₈YYX₉ (SEQ ID NO: 557), wherein X, is V, E, A or G, wherein X₂ is A, F, Y or G, wherein X₃ is G, S, Y or W, wherein X₄ is S or R, wherein X₅ is absent or is N, wherein X₆ is F, S, Y, or absent, wherein X₇ is L or F or absent, wherein Xₐ is D or E, and wherein X₉ is Y, H or S.

45. The method of claim 44, wherein the antigen binding protein comprises:
a. a light chain variable domain comprising:
i. a light chain CDR1 comprising an amino acid sequence RASQSVNSNLA (SEQ ID NO: 556);
ii. a light chain CDR2 comprising an amino acid sequence GASTRAT (SEQ ID NO: 573);
iii. a light chain CDR3 comprising an amino acid sequence QQFKNWPPT (SEQ ID NO: 576); and
b. a heavy chain variable domain comprising:
i. a heavy chain CDR1 comprising an amino acid sequence AYAMS (SEQ ID NO: 581);
ii. a heavy chain CDR2 comprising an amino acid sequence TSGSGSTTYYADSVKG (SEQ ID NO: 584); and
iii. a heavy chain CDR3 comprising an amino acid sequence VAGSNFLFDY (SEQ ID NO: 842).

46. A method of treating cardiovascular disease comprising administering to a subject in need thereof a therapeutically effective amount of an antigen binding protein that binds to TrigGering Receptor Expressed on Myeloid cells 1 (TREM-1) wherein the antigen binding protein comprises;
a. a light chain variable domain comprising:
i. a light chain CDR1 comprising an amino acid sequence QASX₁DIX₂X₃X₄LN (SEQ ID NO: 558), wherein X₁ is R or Q, wherein X₂ is R, S, N or F, wherein X₃ is K or N, and wherein X₄ is H, Y or D;
ii. a light chain CDR2 comprising an amino acid sequence X₁X₂X₃X₄LET (SEQ ID NO: 560), wherein X₁ is D, G or H, wherein X₂ is A, V or T, wherein X₃ is S, A or Y, and wherein X₄ is T or N;
iii. a light chain CDR3 comprising an amino acid sequence QX₁YX₃X₄X₅PX₆T (SEQ ID NO: 561), wherein X₁ is Q or H, wherein X₂ is D, A or G, wherein X₃ is N or K; wherein X₄ is L or I, and wherein X₅ is I or L; and
b. a heavy chain variable domain comprising:
i. a heavy chain CDR1 comprising an amino acid sequence X₁YDIN (SEQ ID NO: 563), wherein X₁ is R or S;
ii. a heavy chain CDR2 comprising an amino acid sequence X₁X₂NPX₃X₄GX₅X₆GX₇X₈ X₉X₁₀FX₁₁X₁₂ (SEQ ID NO: 564), wherein X₁ is W or R, wherein X₂ is M or L, wherein X₃ is N, Q, or K, wherein X₄ is S, A, or R, wherein X₅ is N, or Q, wherein X₆ is S, A, or T, wherein X₇ is S, Q, or Y, wherein X₈ is V or T, wherein X₉ is Q or K, wherein X₁₀ is K or N, wherein X₁₁ is R or Q, and wherein X₁₂ is G or D; and
iii. a heavy chain CDR3 comprising an amino acid sequence X₁X₂X₃X₄ X₅X₆ X₇ X₈ X₉X₁₀X₁₁X₁₂FX₁₃X₁₄ (SEQ ID NO: 565); wherein X₁ is G, L or R, wherein X₂ is G, I, or R, wherein X₃ is Y, R, I, G, or A, wherein X₄ is T, S, Y, or V, wherein X₅ is S or Y, wherein X₆ is S, A, I, or R, wherein X₇ is W, A, or S, wherein X₈ is absent or is S, wherein X₉ is absent or is F, W, or Y, and wherein X₁₀ is R, S, H, K, or E, wherein X₁₁ is W, H, Y, or F, wherein X₁₂ is Y, V, A, or S, wherein X₁₃ is D or Q, and wherein X₁₄ is L, Y, I, or H.

47. The method of claim 46, wherein the antigen binding protein comprises:
a. a light chain variable domain comprising:
i. a light chain CDR1 comprising an amino acid sequence QASQDIRKHLN (SEQ ID NO: 567);
ii. a light chain CDR2 comprising an amino acid sequence DASNLET (SEQ ID NO: 574); and
iii. a light chain CDR3 comprising an amino acid sequence QHYDNLPIT (SEQ ID NO: 577); and
b. a heavy chain variable domain comprising:
i. a heavy chain CDR1 comprising an amino acid sequence RYDIN (SEQ ID NO: 582);
ii. a heavy chain CDR2 comprising an amino acid sequence WMNPNSGNSSVQKFRG (SEQ ID NO: 585); and
iii. a heavy chain CDR3 comprising an amino acid sequence GGYTSSWRWYFDL (SEQ ID NO: 843) or GGYTSSWSRWYFDL (SEQ ID NO: 844).
(SEQ ID NO: 843)
GGYTSSWRWYFDL
or
(SEQ ID NO: 844)
GGYTSSWSRWYFDL.
